(19) United States
(12) Patent Application Publication
(10) Pub. No.: US 2009/0312344 A1
(43) Pub. Date: Dec. 17, 2009

(54) ARYLPIPERAZINE DERIVATIVES AS ADRENERGIC RECEPTOR ANTAGONISTS

(76) Inventors: Mohammad Salman, Princeton, NJ (US); Somesh Sharma, Delhi (IN); Gyan Chand Yadav, Uttar Pradesh (IN); Gobind Singh Kapkoti, Uttaranchal (IN); Anurag Mishra, Delhi (IN); Praful Gupta, Saharanpur Uttar Pradesh (IN); Nitya Anand, Uttar Pradesh (IN); Anita Chugh, Delhi (IN); Kamna Nanda, Delhi (IN)

Correspondence Address: Ranbaxy Inc., Intellectual Property Department, 600 College Road East, PRINCETON, NJ, 08540, US

(21) Appl. No.: 11/569,838

(22) PCT Filed: May 31, 2005
(86) PCT No.: PCT/IB05/01534

§ 371 Date: Apr. 1, 2009

(30) Foreign Application Priority Data
May 31, 2004 (IN) 992/DEL/2004

(51) Int. Cl. A61K 31/496; C07D 403/02; A61P 13/00; C07D 241/04

(52) U.S. Cl. 514/253.12; 544/373; 514/254.09; 544/372; 514/254.01; 544/360; 544/401

(57) ABSTRACT

The present invention relates to α₁, and/or α_{1d} adrenergic receptor antagonists, which can function as α₁ₐ and/or α_{1d} adrenergic receptor antagonist and can be used for the treatment of a disease or disorder mediated through α₁ₐ and/or an adrenergic receptor. Compounds disclosed herein can be used for the treatment of benign prostatic hyperplasia (BPH) and the related symptoms thereof. Further, compounds disclosed herein can be used for the treatment of lower urinary tract symptoms associated with or without BPH. Also provided are processes for preparing such compounds, pharmaceutical compositions thereof, and the methods of treating BPH or related symptoms thereof.

### FIELD OF THE INVENTION

The present invention relates to α₁ₐ and/or α_{1d} adrenergic receptor antagonists, which can be used to treat a disease or disorder mediated through α₁ₐ and/or α_{1d} adrenergic receptors. Compounds and pharmaceutical compositions disclosed herein can be used to treat benign prostatic hyperplasia (BPH) and related symptoms thereof. Further, such compounds can be used to treat lower urinary tract symptoms that may or may not be associated with BPH. The present invention also relates to processes to prepare the disclosed compounds, pharmaceutical compositions thereof, and methods of treating BPH or related symptoms thereof.

### BACKGROUND OF THE INVENTION

Benign prostatic hyperplasia (BPH) is a condition that typically develops in elderly males. BPH causes benign overgrowth of the stromal and epithelial elements of the prostate with aging. Symptoms of BPH can vary and commonly involve changes or problems with urination, such as hesitation, interruption, weak stream, urgency, leaking, dribbling or increased frequency, particularly at night. BPH can consequently cause hypertrophy of bladder smooth muscle, a decompensated bladder or an increased incidence of urinary tract infection.

The symptoms of BPH are a result of two pathological components affecting the prostate gland: a static component and a dynamic component. The static component is related to enlargement of the prostate gland, which may result in compression of the urethra and obstruction to the flow of the urine from the bladder. The dynamic component is related to increased smooth muscle tone of the bladder neck and prostate itself and is regulated by α-1 adrenergic receptor.

Currently, the most effective treatment for BPH is a surgical procedure known as transurethral resection of the prostate (TURP), which involves removing obstructing tissue (C. Chapple, Br. Med. Journal, 304:1198-1199 (1992)). TURP is directed both to the static and dynamic components of the BPH. However, TURP is associated with mortality (1%), adverse events, e.g., incontinence (2-4%), infection (5-10%), and impotence (5-10%). Therefore, noninvasive alternative treatments are highly desirable.

Some drug therapies address the static component of BPH. Administration of finasteride is one such therapy, which is indicated for the treatment of symptomatic BPH. This drug is a competitive inhibitor of the enzyme 5-α reductase that is responsible for the conversion of testosterone to dihydrotestosterone in the prostate gland. Dihydrotestosterone appears to be the major mitogen for prostate growth and agents, which inhibit 5-α reductase, reduce the size of the prostate and improve urine flow through the prostatic urethra. Although finasteride is a potent 5-α reductase inhibitor that causes a marked decrease in serum and tissue concentrations of dihydrotestosterone, it is moderately effective in the treatment of symptomatic BPH. The effects of finasteride take 6-12 months to become evident and for many men the clinical development is minimal.

The dynamic component of BPH has been addressed by the use of adrenergic receptor blocking agents, which act by decreasing the smooth muscle tone within the prostate gland. A variety of α₁ₐ AR antagonists, for example, terazosin, doxazosin, prazosin, alfuzosin and tamulosin, have been investigated for the treatment of symptomatic bladder outlet obstruction due to BPH. However, these drugs are associated with vascular side effects (e.g., postural hypertension, syncope, dizziness, headache etc.) due to lack of selectivity of action between prostatic and vascular α₁ adrenoceptors. There are several lines of evidence suggesting that selectivity for au adrenoceptor over α_{1b} adrenoceptor will result in relative lack of vascular side effects, thus lead to better olerability. Mice deficient in α_{1b} adrenoreceptors show diminished blood pressure response to phenylephrine injection when compared to homozygous controls (decreased blood pressure response in mice deficient of α_{1b} adrenergic receptor. (Proc. Nat'l Acad. Sci. USA, 94:1589-11594 (1997)). In-vivo studies in healthy subjects comparison of α₁ₐ/α_{1d} selective antagonists (e.g., tamsulosin) or α₁ₐ selective antagonists (e.g., urapidil) with non selective antagonists (e.g., doxazosin, prazosin, or terazosin) under a variety of experimental conditions (e.g., involving the administration of exogenous agonist or release of endogenous agonist by cold stimulation) in several vascular beds including the skin circulation in finger tips, the dorsal hand vein, or with total peripheral resistance have been reported. (Eur. J. Clin. Pharmacol., 49:371-375 (1996); N. Schmiedeberg, Arch. Pharmacol., 354:557-561 (1996); Jpn. J. Pharmacol., 80:209-215 (1999); Br. J. Clin. Pharmacol., 47:67-74 (1999)). These studies reported that an antagonist with high affinity for α₁ₐ or α₁ₐ/α_{1d} receptors can cause some degree of vasodilation, although it is much lower than with non-subtype-selective α₁ₐ adrenoceptor antagonists. Further, there is increased vascular α_{1b} adrenoceptor expression in elderly patients and thus α₁ₐ/α_{1d}-selective agents with selectivity over adrenoceptor subtype would be of particular importance in benign prostatic hyperplasia. Antagonism of both α₁ₐ adrenoceptor and α_{1d} adrenoceptor is important to relieve lower urinary tract symptoms especially associated with BPH. Targeting α₁ₐ adrenoceptors with antagonists is important in relaxing prostate smooth muscle and relieving bladder outlet obstruction, whereas α_{1d} adrenoceptor antagonism is important to target irritative symptoms.

In the past decade, there were significant efforts in discovering selective α₁ₐ adrenoceptor antagonists suitable for treating benign prostatic hyperplasia while avoiding cardiovascular side effects that are associated with current drugs. Selective antagonists have been disclosed in, for example, Exp. Opin. Invest. Drugs, 6:367-387 (1997) and J. Med. Chem., 40:1293-1325 (1995). Structure-activity relationships in many of these structural series have been studied in details and numerous highly selective compounds have been identified. There are many description in the literature about the pharmacological activities associated with phenyl piperazines, Eur. J. Med. Chem.-Chimica Therapeutica, 12:173-176 (1977), discloses substituted trifluoromethyl phenyl piperazines having cyclo-imido alkyl side chains shown below.

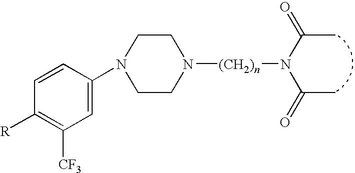

Other related compounds, which have been prepared as anxiolytic, neuroleptic, anti-diabetic and anti-allergic agents, are disclosed in the following references: Yukihiro et al; PCT Appl. WO 98/37893 (1998), Steen et al; J. Med. Chem., 38:4303-4308 (1995), Ishizumi et al., Chem. Pharm. Bult, 39(9):2288-2300 (1991), Kitaro et al.; JP 02-235865 (1990), Ishizumi et al; U.S. Pat. No. 4,598,078 (1086), New et al.; J. Med. Chem., 29:1476-1482 (1986), Shigeru et al; JP 60-204784 (1985), New et al, U.S. Pat. No. 4,524,206 (1985), Korgaonkar et al; J. Indian Chem. Soc., 60:874-876 (1983).

However, α₁ subtype selectivity of the compounds, such as those disclosed in the above-identified references, as well as their usefulness in the treatment of symptoms of benign prostate hyperplasia, were not disclosed in the above references.

The synthesis of 1-(4-arylpiperazin-1-yl)-ω-[N-(α, ω-dicarboximido)]-alkanes useful as uro-selective α₁-adrenoceptor blockers are disclosed in U.S. Pat. Nos. 6,083,950, 6,090,809, 6,410,735, 6,420,559 and 6,420,366, WO 00/05206, US Patent Appl. No. 2002/0156085 and WO 02/44151. These compounds exhibited α₁-adrenergic blocking activity and selectivity.

Other disclosures of selective α₁ₐ adrenoceptor antagonists include U.S. Pat. Nos. 6,376,503, 6,319,932 and 6,339,090, EP 711757, WO 99/42448, WO 99/42445, WO 98/57940, WO 98/57632, WO 98/30560 WO 97/23462, WO 03/084928 and WO 03/084541. Each of these patents are incorporated by reference herein in their entirety.

### SUMMARY OF THE INVENTION

Provided herein are compounds having the structure of Formula I,

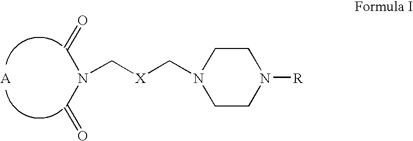

pharmaceutically acceptable salts, pharmaceutically acceptable solvates, enantiomers, diastereomers, N-oxides, prodrugs, polymorphs and metabolites thereof, wherein:

A can be

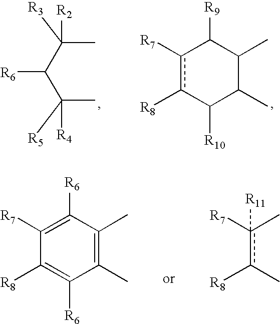

wherein, R₂, R₃, R₄ and R₅ can independently be hydrogen, alkyl or phenyl, R₆ can be hydrogen, alkyl, phenyl, hydroxy or alkoxy, R₇ and R₈ each can independently be hydrogen, alkyl, alkynyl, cycloalkyl, halogen, hydroxy, aryl, acetoxy,
heterocycle,

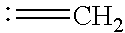

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

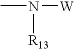

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

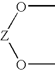

(wherein Z can be CO or SO), R₉ and R₁₀ each can independently be hydrogen, hydroxy, alkoxy, acetyl, or acetyloxy, R₁₁ can be hydrogen, alkyl, alkenyl, alkynyl, cycloalkyl, aryl, or heterocycle, no atom;
X can be CO, CS or CHY (wherein Y can be hydrogen, hydroxy, halogen, alkoxy or haloalkoxy); and
R can be alkyl, alkenyl, alkynyl, cycloalkyl, aryl or heterocycle; with the provisos that
(a) when A is

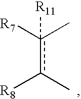

X is —CH₂— and R₁₁ is hydrogen then R₇ can be hydrogen or alkyl with the further proviso that when R₇ is alkyl and R₈ is R₁₂ NH—, then R₁₂ can be substituted alkyl wherein the substituents can be selected from aryl or heterocyclyl,
(b) when A is

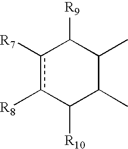

and X is —CH₂—, R₇, R₈, R₉ or R₁₀ are hydrogen or halogen.
(c) When A is

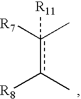

X is —CH₂—, and R₁₁ is no atom, then R7 can be ═CH2.
These compounds can encompass one or more of the following features. For example, A can be

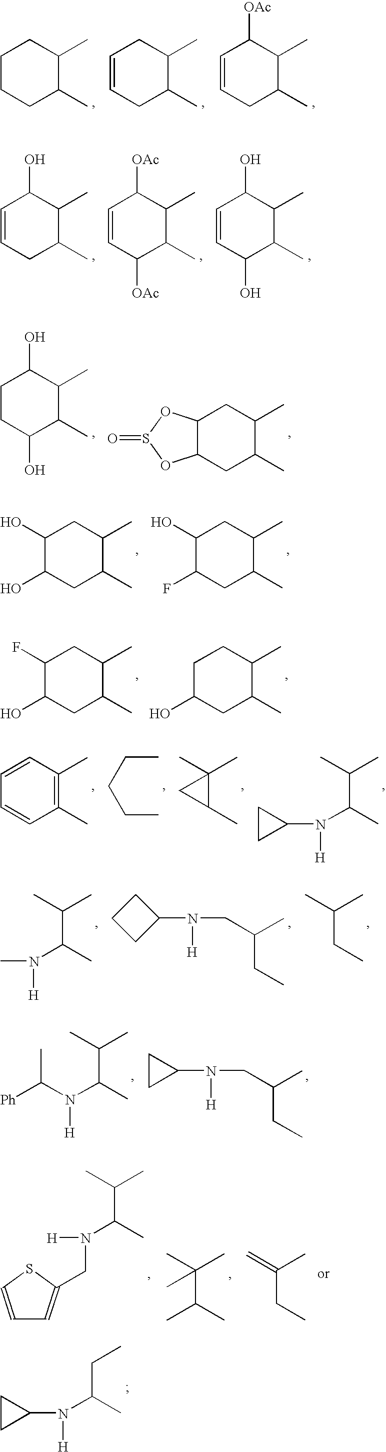

X can be CHOH, CO, CH₂ or CHF; and
R can be: 2-methoxy phenyl, 3-fluoro-2-methoxy phenyl, 5-fluoro-2-methoxy phenyl, 4-fluoro-2-methoxyphenyl, 2-methoxy-5-methyl phenyl, 2-n-propoxyphenyl, 5-fluoro2-n-propoxyphenyl, 2-ethoxy phenyl, 2-isopropoxy phenyl, 4-fluoro-2-isopropoxyphenyl, 4-nitro-2-isopropoxyphenyl, 3-fluoro-2-isopropoxy phenyl, 5-fluoro-2-isopropoxy phenyl, 2-cyclopentoxy-5-fluoro phenyl, 2-cyclopentoxy phenyl, O-tolyl, 2-trifluoroethoxy phenyl, 5-fluoro-2-trifluoromethoxy phenyl or 2-(2,2,3,3-tetrafluoropropoxy)phenyl.

Also provided herein are compounds selected from:

2-{2-Hydroxy-3-[4-(2-isopropoxy-phenyl)-piperazin-1-yl]-propyl}-hexahydro-isoindole-1,3-dione,

2-{2-Hydroxy-3-[4-(2-isopropoxy-phenyl)-piperazin-1-yl]-propyl}-hexahydro-isoindole-1,3-dione hydrochloride salt,

2-{3-[4-(2-Isopropoxy-phenyl)-piperazin-1-yl]-2-oxo-propyl}-hexahydroisoindole-1,3-dione,

2-{3-[4-(2-Isopropoxy-phenyl)-piperazin-1-yl]-2-oxo-propyl}-hexahydroisoindole-1,3-dione hydrochloride salt,

2-{3-[4-(2-Isopropoxy-phenyl)-piperazin-1-yl]-2-oxo-propyl}-3a,4,7,7a-tetrahydro-isoindole-1,3-dione,

2-{3-[4-(2-Isopropoxy-phenyl)-piperazin-1-yl]-2-oxo-propyl}-3a,4,7,7a-tetrahydro-isoindole-1,3-dione hydrochloride salt,

2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione,

2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione hydrochloride salt,

2-(2-Hydroxy-3-{4-[2-(2,2,3,3-tetrafluoro-propoxy)-phenyl]-piperazin-1-yl}-propyl)-3a,4,7,7a-tetrahydro-isoindole-1,3-dione,

2-(2-Hydroxy-3-{4-[2-(2,2,3,3-tetrafluoro-propoxy)-phenyl]-piperazin-1-yl}-propyl)-3a,4,7,7a-tetrahydro-isoindole-1,3-dione hydrochloride salt,

2-{2-Hydroxy-3-[4-(2-isopropoxy-4-nitro-phenyl)-piperazin-1-yl]-propyl}-3a,4,7,7a-tetrahydro-isoindole-1,3-dione,

2-{2-Hydroxy-3-[4-(2-isopropoxy-4-nitro-phenyl)-piperazin-1-yl]-propyl}-3a,4,7,7a-tetrahydro-isoindole-1,3-dione hydrochloride salt,

2-{2-Fluoro-3-[4-(2-isopropoxy-phenyl)-piperazin-1-yl]-propyl}-3a,4,7,7a-tetrahydro-isoindole-1,3-dione,

2-{2-Fluoro-3-[4-(2-isopropoxy-phenyl)-piperazin-1-yl]-propyl}-3a,4,7,7a-tetrahydro-isoindole-1,3-dione hydrochloride salt,

Acetic acid 2-{3-[4-(2-methoxy-phenyl)-piperazin-1-yl]-propyl}-1,3-dioxo-2,3,3a,4,7,7a-hexahydro-1H-inden-4-yl ester,

Acetic acid 2-{3-[4-(2-methoxy-phenyl)-piperazin-1-yl]-propyl}-1,3-dioxo-2,3,3a,4,7,7a-hexahydro-1H-inden-4-yl ester hydrochloride salt,

4-Hydroxy-2-{3-[4-(2-methoxy-phenyl)-piperazin-1-yl]-propyl}-3a,4,7,7a-tetrahydro-isoindole-1,3-dione,

4-Hydroxy-2-{3-[4-(2-methoxy-phenyl)-piperazin-1-yl]-propyl}-3a,4,7,7a-tetrahydro-isoindole-1,3-dione hydrochloride salt,
2-{3-[4-(2-Methoxy-phenyl)-piperazin-1-yl}-2-oxo-propyl}-3a,4,7,7a-tetrahydro-isoindole-1,3-dione,

2-{3-[4-(2-Methoxy-phenyl)-piperazin-1-yl}-2-oxo-propyl}-3a,4,7,7a-tetrahydro-isoindole-1,3-dione hydrochloride salt,

2-{3-[4-(2-Cyclopentyloxy-phenyl)-piperazin-1-yl]-2-oxo-propyl}-3a,4,7,7a-tetrahydro-isoindole-1,3-dione,

2-{2-Oxo-3-[4-(2-propoxy-phenyl)-piperazin-1-yl]-propyl}-3a,4,7,7a-tetrahydro-isoindole-1,3-dione,

2-{2-Oxo-3-[4-(2-propoxy-phenyl)-piperazin-1-yl]-propyl}-3a,4,7,7a-tetrahydro-isoindole-1,3-dione hydrochloride salt,

2-{3-[4-(4-Fluoro-2-isopropoxy-phenyl)-piperazin-1-yl]-2-oxo-propyl}-3a,4,7,7a-tetrahydro-isoindole-1,3-dione,

2-{3-[4-(4-Fluoro-2-isopropoxy-phenyl)-piperazin-1-yl]-2-oxo-propyl}-3a,4,7,7a-tetrahydro-isoindole-1,3-dione hydrochloride salt,

2-{3-[4-(2-Isopropoxy-phenyl)-piperazin-1-yl]-2-oxo-propyl}-isoindole-1,3-dione,

2-{3-[4-(2-Isopropoxy-phenyl)-piperazin-1-yl]-2-oxo-propyl}-isoindole-1,3-dione hydrochloride salt,

2-(2-Oxo-3-{4-[2-(2,2,2-trifluoro-ethoxy)-phenyl]-piperazin-1-yl}-propyl)-3a,4,7,7a-tetrahydro-isoindole-1,3-dione,

2-(2-Oxo-3-{4-[2-(2,2,2-trifluoro-ethoxy)-phenyl]-piperazin-1-yl}-propyl)-3a,4,7,7a-tetrahydro-isoindole-1,3-dione hydrochloride salt,

6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione,

6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione hydrochloride salt,

1-[2-Oxo-3-(4-phenyl-piperazin-1-yl)-propyl]-pyrrolidine-2,5-dione,

1-{3-[4-{4-Fluoro-phenyl}-piperazin-1-yl]-2-oxo-propyl}-3-phenyl-piperidine-2,6-dione,

3,4-Dimethyl-1-{2-oxo-3-[4-(2-trifluoromethyl-phenyl)-piperazin-1-yl]-propyl}-pyrrole-2,5-dione,

1-{2-Fluoro-3-[4-(4-fluorophenyl)piperazin-1-yl]-propyl}-piperidine-2,6-dione,

1-(2-Fluoro-3-{4-[2-(2,2,2-trifluoro-ethoxy)-phenyl] -piperazin-1-yl}propyl)-3,4-dimethylpyrrole-2,5-dione,

1-{3-[4-(2-Cyclopentyloxy-phenyl)-piperazin-1-yl]-2-hydroxy-propyl}-3-cyclopropylamino-4-methyl-pyrrolidine-2,5-dione,

1-{3-[4-(2-Cyclopentyloxy-phenyl)-piperazin-1-yl]-2-hydroxy-propyl}-3-cyclopropylamino-4-methyl-pyrrolidine-2,5-dione hydrochloride salt,

1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione,

1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione hydrochloride salt,

1-{3-[4-(2-Cyclopentyloxy-5-fluoro-phenyl)-piperazin-1-yl]-2-hydroxy-propyl}-3-cyclopropylaminomethyl-pyrrolidine-2,5-dione,

1-{3-[4-(2-Cyclopentyloxy-5-fluoro-phenyl)-piperazin-1-yl]-2-hydroxy-propyl}-3-cyclopropylaminomethyl-pyrrolidine-2,5-dione hydrochloride salt,

2-{3-[4-(5-Fluoro-2-isopropoxy-phenyl)-piperazin-1-yl]-propyl}-5,6-dihydroxy-hexahydro-isoindole-1,3-dione,

2-{3-[4-(5-Fluoro-2-isopropoxy-phenyl)-piperazin-1-yl]-propyl}-5,6-dihydroxy-hexahydro-isoindole-1,3-dione hydrochloride salt,

1-{3-[4-(2-Cyclopentyloxy-5-fluoro-phenyl)-piperazin-1-yl]-2-hydroxy-propyl}-3-methyl-4-methylamino-pyrrolidine-2,5-dione,

1-{3-[4-(2-Cyclopentyloxy-5-fluoro-phenyl)-piperazin-1-yl]-2-hydroxy-propyl}-3-methyl-4-methylamino-pyrrolidine-2,5-dione hydrochloride salt,

1-{3-[4-(2-Methoxy-5-methyl-phenyl)-piperazin-1-yl]-propyl}-piperidine-2,6-dione,

1-{3-[4-(2-Methoxy-5-methyl-phenyl)-piperazin-1-yl]-propyl}-piperidine-2,6-dione hydrochloride salt,

5,6-Dihydroxy-2-{3-[4-(2-methoxy-5-methyl-phenyl)-piperazin-1-yl]-propyl}-hexahydro-isoindole-1,3-dione,

5,6-Dihydroxy-2-{3-[4-(2-methoxy-5-methyl-phenyl)-piperazin-1-yl]-propyl}-hexahydro-isoindole-1,3-dione hydrochloride salt,

1-(3-{4-[5-Fluoro-2-(2,2,2-trifluoro-ethoxy)-phenyl]-piperazin-1-yl}-propyl)-piperidine-2,6-dione,

1-(3-{4-[5-Fluoro-2-(2,2,2-trifluoro-ethoxy)-phenyl]-piperazin-1-yl}-propyl)-piperidine-2,6-dione hydrochloride salt,

2-{3-[4-(2-Cyclopentyloxy-phenyl)-piperazin-1-yl]-propyl}-5,6-dihydroxy-hexahydro-isoindole-1,3-dione,

2-{3-[4-(2-Cyclopentyloxy-phenyl)-piperazin-1-yl]-propyl}-5,6-dihydroxy-hexahydro-isoindole-1,3-dione hydrochloride salt,

3-{3-[4-(3-Fluoro-2-methoxy-phenyl)-piperazin-1-yl]-propyl)-1-methyl-3-aza-bicyclo[3.1.0]hexane-2,4-dione,

3-{3-[4-(3-Fluoro-2-methoxy-phenyl)-piperazin-1-yl]-propyl)-1-methyl-3-aza-bicyclo[3.1.0]hexane-2,4-dione hydrochloride salt,

3-{3-[4-(5-Fluoro-2-methoxy-phenyl)-piperazin-1-yl]-propyl}-1-methyl-3-aza-bicyclo[3.1.0]hexane-2,4-dione,

3-{3-[4-(5-Fluoro-2-methoxy-phenyl)-piperazin-1-yl]-propyl}-1-methyl-3-aza-bicyclo[3.1.0]hexane-2,4-dione hydrochloride salt,

3-{3-[4-(2-Cyclopentyloxy-phenyl)-piperazin-l -yl]-propyl}-1-methyl-3-aza-bicyclo [3.1.0]hexane-2,4-dione,

3-{3-[4-(2-Cyclopentyloxy-phenyl)-piperazin-1-yl]-propyl}-1-methyl-3-aza-bicyclo [3.1.0]hexane-2,4-dione hydrochloride salt,

5-Fluoro-6-hydroxy-2-{2-hydroxy-3-[4-(2-isopropoxy-phenyl)-piperazin-1-yl]-propyl}-hexahydro-isoindole-1,3-dione,

5-Fluoro-6-hydroxy-2-{2-hydroxy-3-[4-(2-isopropoxy-phenyl)-piperazin-1-yl]-propyl}-hexahydro-isoindole-1,3-dione hydrochloride salt,

3-{3-[4-(5-Fluoro-2-isopropoxy-phenyl)-piperazin-1-yl]-propyl}-1-methyl-3-aza-bicyclo[3.1.0]hexane-2,4-dione,

3-{3-[4-(5-Fluoro-2-isopropoxy-phenyl)-piperazin-1-yl]-propyl}-1-methyl-3-aza-bicyclo[3.1.0]hexane-2,4-dione hydrochloride salt,

5-Fluoro-6-hydroxy-2-{3-[4-(2-isopropoxy-phenyl)-piperazin-1-yl]-propyl}-hexahydro-isoindole-1,3-dione,

5-Fluoro-6-hydroxy-2-{3-[4-(2-isopropoxy-phenyl)-piperazin-1-yl]-propyl}-hexahydro-isoindole-1,3-dione hydrochloride salt,

2-{3-[4-(2-Cyclopentyloxy-phenyl)-piperazin-1-yl]-2-hydroxy-propyl}-hexahydro-isoindole-1,3-dione,

2-{3-[4-(2-Cyclopentyloxy-phenyl)-piperazin-1-yl]-2-hydroxy-propyl}-hexahydro-isoindole-1,3-dione hydrochloride salt,

5-Hydroxy-2-{3-[4-(2-methoxy-phenyl)-piperazin-1-yl]-propyl}-hexahydro-isoindole-1,3-dione,

5-Hydroxy-2-{3-[4-(2-methoxy-phenyl)-piperazin-1-yl]-propyl}-hexahydro-isoindole-1,3-dione hydrochloride salt,

2-{3-[4-(2-Cyclopentyloxy-5-fluoro-phenyl)-piperazin-1-yl]-propyl}-5-hydroxy-hexahydro-isoindole-1,3-dione,

2-{3-[4-(2-Cyclopentyloxy-5-fluoro-phenyl)-piperazin-1-yl]-propyl}-5-hydroxy-hexahydro-isoindole-1,3-dione hydrochloride salt,

2-{3-[4-(2-Cyclopentyloxy-phenyl)-piperazin-1-yl]-propyl}-5-hydroxy-hexahydro-isoindole-1,3-dione,

2-{3-[4-(2-Cyclopentyloxy-phenyl)-piperazin-1-yl]-propyl}-5-hydroxy-hexahydro-isoindole-1,3-dione hydrochloride salt,

2-{3-[4-(2-Cyclopentyloxy-phenyl)-piperazin-1-yl]-2-oxo-propyl}-5,6-dihydroxy-hexahydro-isoindole-1,3-dione,

2-{3-[4-(2-Cyclopentyloxy-phenyl)-piperazin-1-yl]-2-oxo-propyl}-5,6-dihydroxy-hexahydro-isoindole-1,3-dione hydrochloride salt,

2-{3-[4-(2-Cyclopentyloxy-5-fluoro-phenyl)-piperazin-1-yl]-2-oxo-propyl}-3a,4,7,7a-tetrahydro-isoindole-1,3-dione,

2-{3-[4-(2-Cyclopentyloxy-5-fluoro-phenyl)-piperazin-1-yl]-2-oxo-propyl}-3a,4,7,7a-tetrahydro-isoindole-1,3-dione hydrochloride salt,

2-{3-[4-(2-Cyclopentyloxy-5-fluoro-phenyl)-piperazin-1-yl]-2-hydroxy-propyl}-hexahydro-isoindole-1,3-dione,

2-{3-[4-(2-Cyclopentyloxy-5-fluoro-phenyl)-piperazin-1-yl]-2-hydroxy-propyl}-hexahydro-isoindole-1,3-dione hydrochloride salt,

5-Fluoro-2-{3-[4-(5-fluoro-2-methoxy-phenyl)-piperazin-1-yl]-propyl}-6-hydroxy-hexahydro-isoindole-1,3-dione,

5-Fluoro-2-{3-[4-(5-fluoro-2-methoxy-phenyl)-piperazin-1-yl]-propyl}-6-hydroxy-hexahydro-isoindole-1,3-dione hydrochloride salt,

2-{3-[4-(5-Fluoro-2-isopropoxy-phenyl)-piperazin-1-yl]-propyl}-5-hydroxy-hexahydro-isoindole-1,3-dione,

2-{3-[4-(5-Fluoro-2-isopropoxy-phenyl)-piperazin-l -yl]-propyl}-5-hydroxy-hexahydro-isoindole-1,3-dione hydrochloride salt,

5-Fluoro-2-{3-[4-(5-fluoro-2-isopropoxy-phenyl)-piperazin-1-yl]-propyl}-6-hydroxy-hexahydro-isoindole-1,3-dione,

5-Fluoro-2-{3-[4-(5-fluoro-2-isopropoxy-phenyl)-piperazin-1-yl]-propyl}-6-hydroxy-hexahydro-isoindole-1,3-dione hydrochloride salt,

1-{3-[4-(5-Fluoro-2-isopropoxy-phenyl)-piperazin-1-yl]-2-hydroxy-propyl}-piperidine-2,6-dione,

1-{3-[4-(5-Fluoro-2-isopropoxy-phenyl)-piperazin-1-yl]-2-hydroxy-propyl}-piperidine-2,6-dione hydrochloride salt,

1-{3-[4-(2-Cyclopentyloxy-5-fluoro-phenyl)-piperazin-1-yl]-2-hydroxy-propyl}-piperidine-2,6-dione,

1-{3-[4-(2-Cyclopentyloxy-5-fluoro-phenyl)-piperazin-1-yl]-2-hydroxy-propyl}-piperidine-2,6-dione hydrochloride salt,

Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester,

Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester hydrochloride salt,

Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester,

Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester hydrochloride salt,

2-{3-[4-(5-Fluoro-2-isopropoxy-phenyl)-piperazin-l -yl]-propyl}-4,7-dihydroxy-3a,4,7,7a-tetrahydro-isoindole-1,3-dione,

2-{3-[4-(5-Fluoro-2-isopropoxy-phenyl)-piperazin-1-yl]-propyl}-4,7-dihydroxy-3a,4,7,7a-tetrahydro-isoindole-1,3-dione hydrochloride salt,

1-{3-[4-(5-Fluoro-2-propoxy-phenyl)-piperazin-1-yl]-propyl}-piperidine-2,6-dione,

1-{3-[4-(5-Fluoro-2-propoxy-phenyl)-piperazin-1-yl]-propyl}-piperidine-2,6-dione hydrochloride salt,

1-{3-[4-(5-Fluoro-2-propoxy-phenyl)-piperazin-1-yl]-2-hydroxy-propyl)-piperidine-2,6-dione,

1-{3-[4-(5-Fluoro-2-propoxy-phenyl)-piperazin-1-yl]-2-hydroxy-propyl)-piperidine-2,6-dione hydrochloride salt,

2-{3-[4-(2-Cyclopentyloxy-5-fluoro-phenyl)-piperazin-1-yl]-propyl}-5,6-dihydroxy-hexahydro-isoindole-1,3-dione,

2-{3-[4-(2-Cyclopentyloxy-5-fluoro-phenyl)-piperazin-1-yl]-propyl}-5,6-dihydroxy-hexahydro-isoindole-1,3-dione hydrochloride salt,

2-{3-[4-(5-Fluoro-2-propoxy-phenyl)-piperazin-1-yl]-propyl}-5,6-dihydroxy-hexahydro-isoindole-1,3-dione,

2-{3-[4-(5-Fluoro-2-propoxy-phenyl)-piperazin-1-yl]-propyl}-5,6-dihydroxy-hexahydro-isoindole-1,3-dione hydrochloride salt,

2-{3-[4-(2-Cyclopentyloxy-phenyl)-piperazin-1-yl]-propyl}-5-fluoro-6-hydroxy-hexahydro-isoindole-1,3-dione,

2-{3-[4-(2-Cyclopentyloxy-phenyl)-piperazin-1-yl]-propyl}-5-fluoro-6-hydroxy-hexahydro-isoindole-1,3-dione hydrochloride salt,

2-{3-[4-(2-Cyclopentyloxy-5-fluoro-phenyl)-piperazin-1-yl]-propyl}-5-fluoro-6-hydroxy-hexahydro-isoindole-1,3-dione,

2-{3-[4-(2-Cyclopentyloxy-5-fluoro-phenyl)-piperazin-1-yl]-propyl}-5-fluoro-6-hydroxy-hexahydro-isoindole-1,3-dione hydrochloride salt,

3-Cyclopropylaminomethyl-1-{2-hydroxy-3-[4-(2-methoxy-phenyl)-piperazin-1-yl]-propyl}-pyrrolidine-2,5-dione,

3-Cyclopropylaminomethyl-1-{2-hydroxy-3-[4-(2-methoxy-phenyl)-piperazin-1-yl]-propyl}-pyrrolidine-2,5-dione hydrochloride salt,

3-Cyclopropylaminomethyl-1-{2-hydroxy-3-[4-(2-methoxy-phenyl)-piperazin-1-yl]-propyl}-4-methyl-pyrrolidine-2,5-dione,

3-Cyclopropylaminomethyl-1-{2-hydroxy-3-[4-(2-methoxy-phenyl)-piperazin-1-yl]-propyl}-4-methyl-pyrrolidine-2,5-dione hydrochloride salt,

3-Cyclobutylaminomethyl-1-{2-hydroxy-3-[4-(2-methoxy-phenyl)-piperazin-1-yl]-propyl}-pyrrolidine-2,5-dione,

3-Cyclobutylaminomethyl-1-{2-hydroxy-3-[4-(2-methoxy-phenyl)-piperazin-1-yl]-propyl}-pyrrolidine-2,5-dione hydrochloride salt,

1-{2-Hydroxy-3-[4-(2-methoxy-phenyl)-piperazin-1-yl]-propyl}-3-methyl-pyrrolidine-2,5-dione,

1-{2-Hydroxy-3-[4-(2-methoxy-phenyl)-piperazin-1-yl]-propyl}-3-methyl-pyrrolidine-2,5-dione hydrochloride salt,

2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione,

2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione hydrochloride salt,

2-{3-[4-(2-Cyclopentyloxy-5-fluoro-phenyl)-piperazin-1-yl]-2-oxo-propyl}-5,6-dihydroxy-hexahydro-isoindole-1,3-dione,

2-{3-[4-(2-Cyclopentyloxy-5-fluoro-phenyl)-piperazin-1-yl]-2-oxo-propyl}-5,6-dihydroxy-hexahydro-isoindole-1,3-dione hydrochloride salt,

2-{3-[4-(5-Fluoro-2-isopropoxy-phenyl)-piperazin-1-yl]-2-oxo-propyl}-5,6-dihydroxy-hexahydro-isoindole-1,3-dione,

2-{3-[4-(5-Fluoro-2-isopropoxy-phenyl)-piperazin-1-yl]-2-oxo-propyl}-5,6-dihydroxy-hexahydro-isoindole-1,3-dione hydrochloride salt,

2-{3-[4-(5-Fluoro-2-isopropoxy-phenyl)-piperazin-1-yl]-2-hydroxy-propyl}-5,6-dihydroxy-hexahydro-isoindole-1,3-dione,

2-{3-[4-(5-Fluoro-2-isopropoxy-phenyl)-piperazin-l -yl]-2-hydroxy-propyl}-5,6-dihydroxy-hexahydro-isoindole-1,3-dione hydrochloride salt,

2-{3-[4-(2-Cyclopentyloxy-5-fluoro-phenyl)-piperazin-1-yl]-2-hydroxy-propyl}-5,6-dihydroxy-hexahydro-isoindole-1,3-dione,

2-{3-[4-(2-Cyclopentyloxy-5-fluoro-phenyl)-piperazin-1-yl]-2-hydroxy-propyl}-5,6-dihydroxy-hexahydro-isoindole-1,3-dione hydrochloride salt,

1-{3-[4-(2-Cyclopentyloxy-5-fluoro-phenyl)-piperazin-1-yl]-propyl}-piperidine-2,6-dione,

1-{3-[4-(2-Cyclopentyloxy-5-fluoro-phenyl)-piperazin-1-yl]-propyl}-piperidine-2,6-dione hydrochloride salt,

1-{3-[4-(3-Fluoro-2-isopropoxy-phenyl)-piperazin-1-yl]-propyl}-piperidine-2,6-dione,

1-{3-[4-(3-Fluoro-2-isopropoxy-phenyl)-piperazin-1-yl]-propyl}-piperidine-2,6-dione hydrochloride salt,

1-{3-[4-(3-Fluoro-2-methoxy-phenyl)-piperazin-1-yl]-propyl}-piperidine-2,6-dione,

1-{3-[4-(3-Fluoro-2-methoxy-phenyl)-piperazin-1-yl]-propyl}-piperidine-2,6-dione hydrochloride salt,

1-{3-[4-(3-Fluoro-2-isopropoxy-phenyl)-piperazin-1-yl]-propyl}-3-methylene-pyrrolidine-2,5-dione,

1-{3-[4-(3-Fluoro-2-isopropoxy-phenyl)-piperazin-l -yl]-propyl}-3-methylene-pyrrolidine-2,5-dione hydrochloride salt,

1-{3-[4-(5-Fluoro-2-isopropoxy-phenyl)-piperazin-1-yl]-propyl}-3-methylene-pyrrolidine-2,5-dione,

1-{3-[4-(5-Fluoro-2-isopropoxy-phenyl)-piperazin-1-yl]-propyl}-3-methylene-pyrrolidine-2,5-dione hydrochloride salt,

1-{3-[4-(5-Fluoro-2-(2,2,3,3-tetrafluoro-propoxy)-phenyl]-piperazin-1-yl}-propyl)-piperidine-2,6-dione,

1-{3-[4-(5-Fluoro-2-(2,2,3,3-tetrafluoro-propoxy)-phenyl]-piperazin-1-yl}-propyl)-piperidine-2,6-dione hydrochloride salt,

1-{3-[4-(2-Methoxy-phenyl)-piperazin-1-yl]-propyl)-3-methyl-4-(1-phenyl-ethylamino)-pyrrolidine-2,5-dione,

1-{(3-[4-(2-Methoxy-phenyl)-piperazin-1-yl]-propyl)-3-methyl-4-(1-phenyl-ethylamino)-pyrrolidine-2,5-dione hydrochloride salt,

1-{3-[4-(5-Fluoro-2-trifluoromethoxy-phenyl)-piperazin-1-yl]-propyl)-piperidine-2,6-dione,

1-{3-[4-(5-Fluoro-2-trifluoromethoxy-phenyl)-piperazin-1-yl]-propyl)-piperidine-2,6-dione hydrochloride salt,

Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-yl ester,

Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-yl ester hydrochloride salt,

2-{3-[4-(2-Ethoxy-phenyl)-piperazin-1-yl]-propyl)-4,7-dihydroxy-3a,4,7,7a-tetrahydro-isoindole-1,3-dione,

2-{3-[4-(2-Ethoxy-phenyl)-piperazin-1-yl]-propyl)-4,7-dihydroxy-3a,4,7,7a-tetrahydro-isoindole-1,3-dione hydrochloride salt,

3-Cyclopropylamino-1-{3-[4-(2-ethoxy-phenyl)-piperazin-1-yl]-propyl}-pyrrolidine-2,5-dione,

3-Cyclopropylamino-1-{3-[4-(2-ethoxy-phenyl)-piperazin-1-yl]-propyl}-pyrrolidine-2,5-dione hydrochloride salt,

Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester,

Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester hydrochloride salt,

1-{3-[4-(2-Cyclopentyloxy-phenyl)-piperazin-1-yl]-propyl)-3-cyclopropylamino-pyrrolidine-2,5-dione,

1-{3-[4-(2-Cyclopentyloxy-phenyl)-piperazin-1-yl]-propyl)-3-cyclopropylamino-pyrrolidine-2,5-dione hydrochloride salt,

4,7-Dihydroxy-2-{3-[4-(2-methoxy-phenyl)-piperazin-1-yl]-propyl)-3a,4,7,7a-tetrahydro-isoindole-1,3-dione,

4,7-Dihydroxy-2-{3-[4-(2-methoxy-phenyl)-piperazin-1-yl]-propyl)-3a,4,7,7a-tetrahydro-isoindole-1,3-dione hydrochloride salt,

Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester,

Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester hydrochloride salt,

2-{3-[4-(2-Cyclopentyloxy-phenyl)-piperazin-1-yl]-propyl)-4,7-dihydroxy-hexahydro-isoindole-1,3-dione,

2-{3-[4-(2-Cyclopentyloxy-phenyl)-piperazin-1-yl]-propyl)-4,7-dihydroxy-hexahydro-isoindole-1,3-dione hydrochloride salt,

2-{3-[4-(2-Cyclopentyloxy-phenyl)-piperazin-1-yl]-propyl}-4,7-dihydroxy-3a,4,7,7a-tetrahydro-isoindole-1,3-dione,

2-{3-[4-(2-Cyclopentyloxy-phenyl)-piperazin-1-yl]-propyl}-4,7-dihydroxy-3a,4,7,7a-tetrahydro-isoindole-1,3-dione hydrochloride salt,

3-Methylene-1-[3-(4-O-tolyl-piperazin-1-yl)-propyl]-pyrrolidine-2,5-dione,

3-Methylene-1-[3-(4-O-tolyl-piperazin-1-yl)-propyl]-pyrrolidine-2,5-dione hydrochloride salt,

1-{3-[4-(2-Methoxy-phenyl)-piperazin-1-yl]-propyl)-3-methyl-4-[(thiophen-2-ylmethyl)-amino]-pyrrolidine-2,5-dione,

1-{3-[4-(2-Methoxy-phenyl)-piperazin-1-yl]-propyl)-3-methyl-4-[(thiophen-2-ylmethyl)-amino]-pyrrolidine-2,5-dione hydrochloride salt,

1-{3-[4-(2-Cyclopentyloxy-phenyl)-piperazin-1-yl]-propyl}-3-methylene-pyrrolidine-2,5-dione,

1-{3-[4-(2-Cyclopentyloxy-phenyl)-piperazin-1-yl]-propyl}-3-methylene-pyrrolidine-2,5-dione hydrochloride salt,

1-{3-[4-(5-Fluoro-2-methoxy-phenyl)-piperazin-1-yl]-propyl}-3,3,4-trimethyl-pyrrolidine-2,5-dione,

1-{3-[4-(5-Fluoro-2-methoxy-phenyl)-piperazin-1-yl]-propyl}-3,3,4-trimethyl-pyrrolidine-2,5-dione hydrochloride salt,

1-{3-[4-(2-Cyclopentyloxy-phenyl)-piperazin-1-yl]-propyl}-piperidine-2,6-dione,

1-{3-[4-(2-Cyclopentyloxy-phenyl)-piperazin-1-yl]-propyl}-piperidine-2,6-dione hydrochloride salt, or

their pharmaceutically acceptable salts, pharmaceutically acceptable solvates, enantiomers, diastereomers, N-oxides, prodrugs, polymorphs or metabolites.

Also provided herein are pharmaceutical compositions comprising a therapeutically effective amount of a compound disclosed herein and optionally one or more pharmaceutically acceptable carriers, excipients or diluents.

Also provided herein are methods for treating a disease or disorder mediated through α₁ₐ and/or α_{1d} adrenergic receptors, comprising administering to patient in need thereof a therapeutically effective amount of a compound disclosed herein and optionally one or more pharmaceutically acceptable carriers, excipients or diluents. These methods can encompass one or more of the following features. For example, the disease or disorder can be benign prostatic hyperplasia. In another example, the compound causes minimal decrease or no decrease in blood pressure at dosages effective to alleviate benign prostatic hyperplasia.

Also provided herein are methods for treating lower urinary tract symptoms associated with or without benign prostatic hyperplasia, comprising administering to a patient in need thereof a therapeutically effective amount of a compound disclosed herein and optionally one or more pharmaceutically acceptable carriers, excipients or diluents.

Also provided herein are methods for preparing compounds of Formula VII,

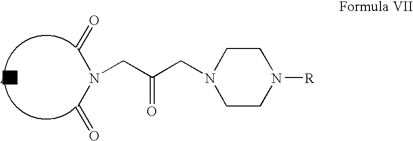

pharmaceutically acceptable salts, pharmaceutically acceptable solvates, enantiomers, diastereomers, N-oxides, prodrugs, polymorphs and metabolites thereof,
wherein A can be

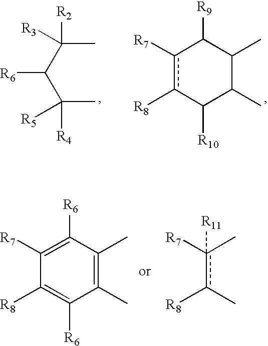

wherein, R₂, R₃, R₄ and R₅ each can independently be hydrogen, alkyl or phenyl, R₆ can be hydrogen, alkyl, phenyl, hydroxy or alkoxy, R₇ and R₈ each can independently be hydrogen, alkyl, alkynyl, cycloalkyl, halogen, hydroxy, aryl, acetoxy, heterocycle,

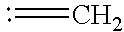

(wherein [symbol] can be the point of attachment) or R₁₂-Q-(CH₂)ₘ— (wherein mean be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

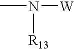

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

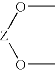

(wherein Z can be CO or SO), R₉ and R₁₀ each can independently be hydrogen, hydroxy, alkoxy, acetyl, or acetyloxy, R₁₁ can be no atom hydrogen, alkyl, alkenyl, alkynyl, cycloalkyl, aryl, or heterocycle;
X can be CO, CS or CHY (wherein Y can be hydrogen, hydroxy, halogen, alkoxy or haloalkoxy); and
R can be alkyl, alkenyl, alkynyl, cycloalkyl, aryl or heterocycle; with the provisos that
(i) when A is

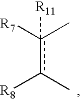

X is —CH₂— and R₁₁ is hydrogen then R₇ can be hydrogen or alkyl with the further proviso that when R₇ is alkyl and R₈ is R₁₂ NH—, then R₁₂ can be substituted alkyl wherein the substituents can be selected from aryl or heterocyclyl,
(ii) when A is

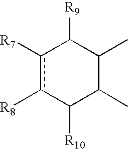

and X is —CH₂—, then none of R₇, R₈, R₉ or R₁₀ are hydrogen or halogen.
(iii) when A is

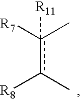

X is —CH₂—, and R₁₁ is no atom, then R₇ can be

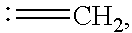

(a) reacting a compound of Formula II

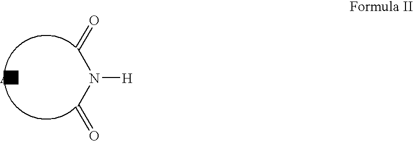

with 2-chloromethyl-oxirane

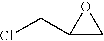

to form a compound of Formula III,

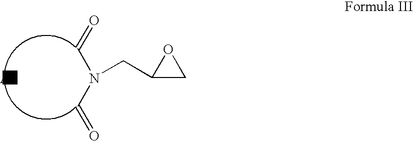

(b) reacting a compound of Formula III with hydrochloric acid to form a compound of Formula IV,

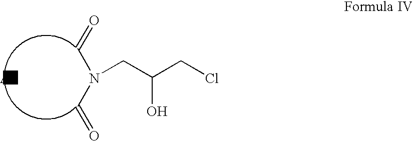

(c) oxidizing a compound of Formula IV to form a compound of Formula V,

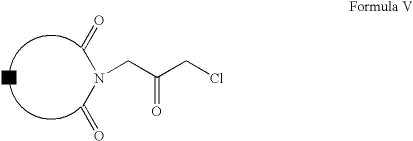

(d) treating a compound of Formula V with a compound of Formula VI

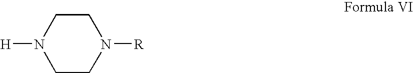

to form a compound of Formula VII.

Also provided herein are methods for preparing compounds of Formula VIII,

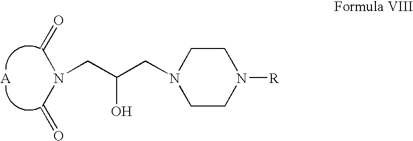

pharmaceutically acceptable salts, pharmaceutically acceptable solvates, enantiomers, diastereomers, N-oxides, prodrugs, polymorphs or metabolites thereof,
wherein A can be

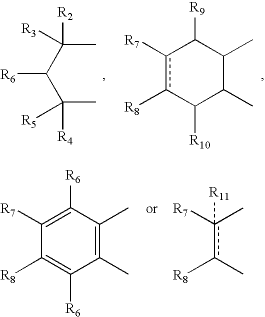

wherein, R₂, R₃, R₄ and R₅ each can independently be hydrogen, alkyl or phenyl, R₆ can be hydrogen, alkyl, phenyl, hydroxy or alkoxy, R₇ and R₈ each can independently be hydrogen, alkyl, alkynyl, cycloalkyl, halogen, hydroxy, aryl, acetoxy, heterocycle,

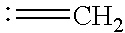

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

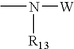

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

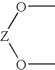

(wherein Z can be CO or SO), R₉ and R₁₀ each can independently be hydrogen, hydroxy, alkoxy, acetyl, or acetyloxy, R₁₁ can be no atom hydrogen, alkyl, alkenyl, alkynyl, cycloalkyl, aryl, or heterocycle;
X can be CO, CS or CHY (wherein Y can be hydrogen, hydroxy, halogen, alkoxy or haloalkoxy); and
R can be alkyl, alkenyl, alkynyl, cycloalkyl, aryl or heterocycle;
with the provisos that
(i) when A is

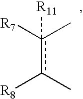

X is —CH₂— and R₁₁ is hydrogen then R₇ can be hydrogen or alkyl with the further proviso that when R₇ is alkyl and R₈ is R₁₂ NH—, then R₁₂ can be substituted alkyl wherein the substituents can be selected from aryl or heterocyclyl,
(ii) when A is and

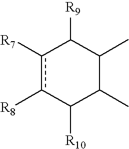

X is —CH₂—, then none of R₇, R₈, R₉ or R₁₀ are hydrogen or halogen,
(iii) when A is

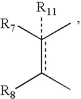

X is —CH₂—, and R₁₁ is no atom, then R₇ can be

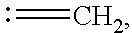

(a) reacting a compound of Formula III

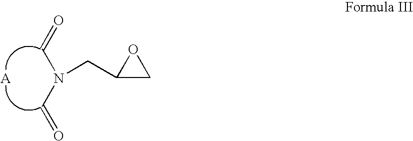

with a compound of Formula VI

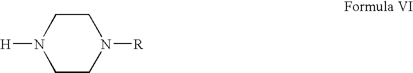

to form a compound of Formula VIII.

Also provided herein are methods for preparing a compound of Formula VII,

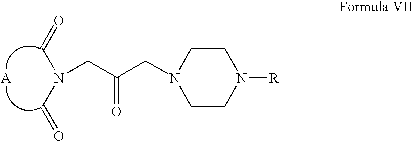

pharmaceutically acceptable salts, pharmaceutically acceptable solvates, enantiomers, diastereomers, N-oxides, prodrugs, polymorphs and metabolites thereof,
wherein A can be

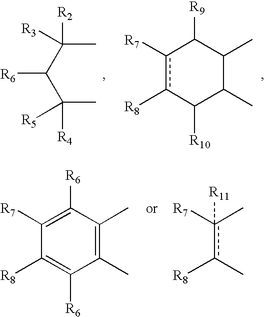

wherein, R₂, R₃, R₄ and R₅ each can independently be hydrogen, alkyl or phenyl, R₆ can be hydrogen, alkyl, phenyl, hydroxy or alkoxy, R₇ and R₈ each can independently be hydrogen, alkyl, alkynyl, cycloalkyl, halogen, hydroxy, aryl, acetoxy, heterocycle,

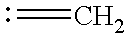

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

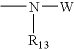

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

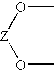

(wherein Z can be CO or SO), R₉ and R₁₀ each can independently be hydrogen, hydroxy, alkoxy, acetyl, or acetyloxy, R₁₁ can be no atom hydrogen, alkyl, alkenyl, alkynyl, cycloalkyl, aryl, or heterocycle;
X can be CO, CS or CHY (wherein Y can be hydrogen, hydroxy, halogen, alkoxy or haloalkoxy); and
R can be alkyl, alkenyl, alkynyl, cycloalkyl, aryl or heterocycle;
with the provisos that
(i) when A is

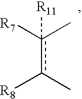

X is —CH₂— and R₁₁ is hydrogen then R₇ can be hydrogen or alkyl with the further proviso that when R₇ is alkyl and R₈ is R₁₂ NH—, then R₁₂ can be substituted alkyl wherein the substituents can be selected from aryl or heterocyclyl,
(ii) when A is

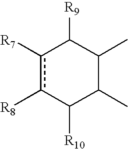

and X is —CH₂—, then none of R₇, R₈, R₉ or R₁₀ are hydrogen or halogen, which method comprises:
(iii) when A is

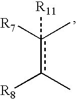

X is —CH₂—, and R₁₁ is no atom, then R₇ can be

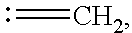

oxidising a compound of Formula VIII

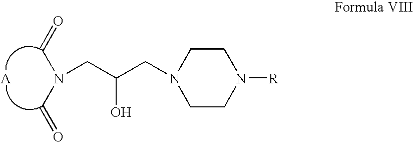

to form a compound of Formula VII.

Also provided herein are methods for preparing compounds of Formula IX,

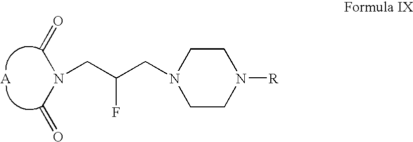

pharmaceutically acceptable salts, pharmaceutically acceptable solvates, enantiomers, diastereomers, N-oxides, prodrugs, polymorphs and metabolites thereof,
wherein A can be

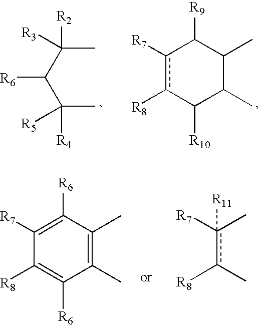

wherein, R₂, R₃, R₄ and R₅ each can independently be hydrogen, alkyl or phenyl, R₆ can be hydrogen, alkyl, phenyl, hydroxy or alkoxy, R₇ and R₈ each can independently be hydrogen, alkyl, alkynyl, cycloalkyl, halogen, hydroxy, aryl, acetoxy, heterocycle,

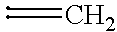

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

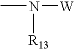

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

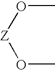

(wherein Z can be CO or SO), R₉ and R₁₀ each can independently be hydrogen, hydroxy, alkoxy, acetyl, or acetyloxy, R₁₁ can be no atom hydrogen, alkyl, alkenyl, alkynyl, cycloalkyl, aryl, or heterocycle;
X can be CO, CS or CHY (wherein Y can be hydrogen, hydroxy, halogen, alkoxy or haloalkoxy); and
R can be alkyl, alkenyl, alkynyl, cycloalkyl, aryl or heterocycle;
with the provisos that
(i) when A is

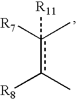

X is —CH₂— and R₁₁ is hydrogen then R₇ can be hydrogen or alkyl with the further proviso that when R₇ is alkyl and R₈ is R₁₂ NH—, then R₁₂ can be substituted alkyl wherein the substituents can be selected from aryl or heterocyclyl,
(ii) when A is

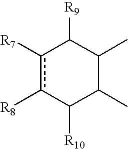

and X is —CH₂—, then none of R₇, R₈, R₉ or R₁₀ are hydrogen or halogen,
(iii) when A is

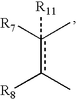

X is —CH₂—, and R₁₁ is no atom, then R₇ can be

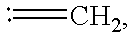

which method comprises:
   fluorinating a compound of Formula VIII

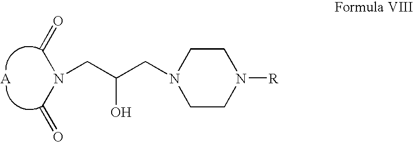

to form a compound of Formula IX.

Also provided herein are methods for preparing compounds of Formula XII,

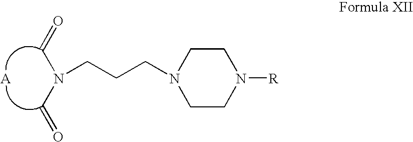

pharmaceutically acceptable salts, pharmaceutically acceptable solvates, enantiomers, diastereomers, N-oxides, prodrugs, polymorphs and metabolites thereof,
wherein A can be

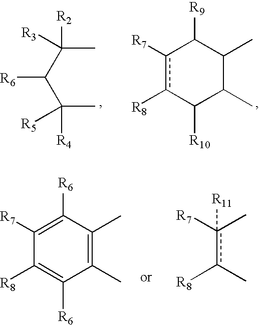

wherein, R₂, R₃, R₄ and R₅ each can independently be hydrogen, alkyl or phenyl, R₆ can be hydrogen, alkyl, phenyl, hydroxy or alkoxy, R₇ and R₈ each can independently be hydrogen, alkyl, alkynyl, cycloalkyl, halogen, hydroxy, aryl, acetoxy, heterocycle,

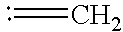

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

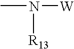

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

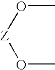

(wherein Z can be CO or SO), R₉ and R₁₀ each can independently be hydrogen, hydroxy, alkoxy, acetyl, or acetyloxy, R₁₁ can be no atom hydrogen, alkyl, alkenyl, alkynyl, cycloalkyl, aryl, or heterocycle;
X can be CO, CS or CHY (wherein Y can be hydrogen, hydroxy, halogen, alkoxy or haloalkoxy); and
R can be alkyl, alkenyl, alkynyl, cycloalkyl, aryl or heterocycle;
with the provisos that
(i) when A is

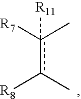

X is —CH₂— and R₁₁ is hydrogen then R₇ can be hydrogen or alkyl with the further proviso that when R₇ is alkyl and R₈ is R₁₂ NH—, then R₁₂ can be substituted alkyl wherein the substituents can be selected from aryl or heterocyclyl,
(ii) when A is and

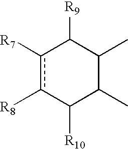

X is —CH₂—, R₇, R₈, R₉ or R₁₀ are hydrogen or halogen,
which method comprises:
   (a) alkylating a compound of Formula II with a compound of Formula X

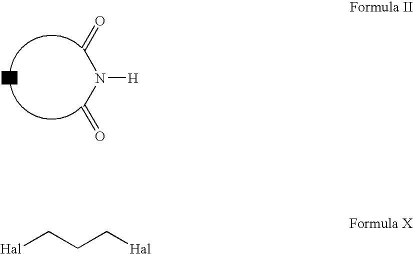

to form a compound of Formula XI

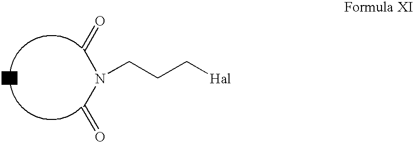

(b) reacting a compound of Formula XI with a compound of VI

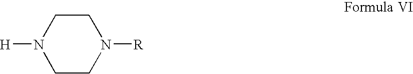

to form a compound of Formula XII.

Also provided herein are methods for preparing compounds of Formula XVI,

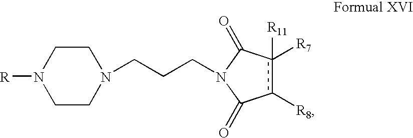

pharmaceutically acceptable salts, pharmaceutically acceptable solvates, enantiomers, diastereomers, N-oxides, prodrugs, polymorphs and metabolites thereof,
wherein R₇ and R₈ each can independently be hydrogen, alkyl, alkynyl, cycloalkyl, halogen, hydroxy, aryl, acetoxy, heterocycle,

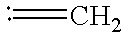

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

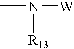

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

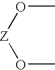

(wherein Z can be CO or SO), R₁₁ can be, no atom hydrogen, alkyl, alkenyl, alkynyl, cycloalkyl, aryl, or heterocycle; and
R can be alkyl, alkenyl, alkynyl, cycloalkyl, aryl or heterocycle;
which method comprises:
   (a) reacting a compound of Formula VI

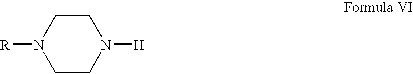

with acrylonitrile to form a compound of Formula XIII,

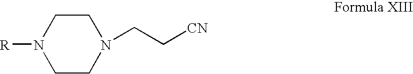

(b) reducing a compound of Formula XIII to form a compound of Formula XIV, and

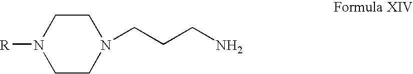

(c) reacting a compound of Formula XIV with a compound of Formula XV

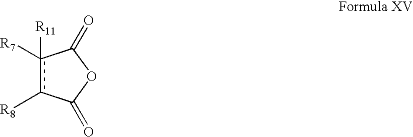

to form a compound of Formula XVI.

Also provided herein are methods for preparing compounds of Formula XVIII,

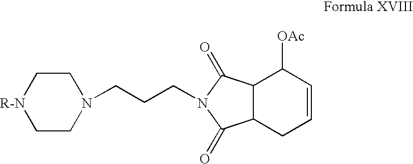

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises:
   reacting a compound of Formula XVII

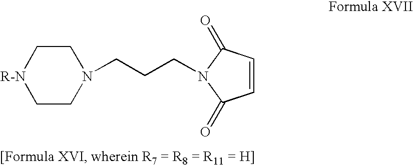

with 1-acetoxy-1,3-butadiene to form a compound of Formula XVIII.

Also provided herein are methods for preparing compounds of Formula XIX,

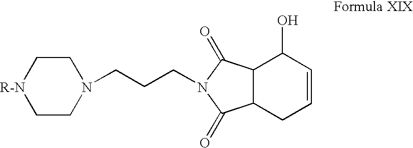

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises:
   hydrolyzing a compound of Formula XVIII

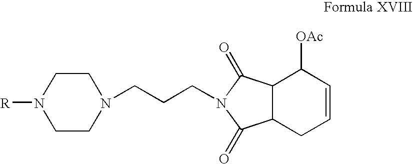

to form a compound of Formula XIX.

Also provided herein are methods for preparing compounds of Formula XX,

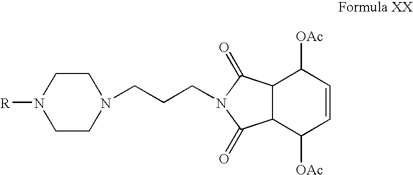

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises:
   reacting a compound of Formula XVII

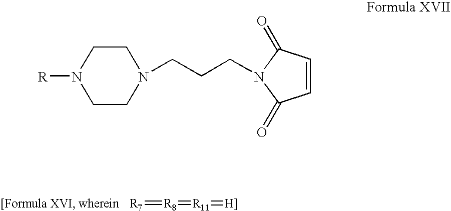

with 1,4-diacetoxy-1,3-butadiene to form a compound of Formula XX.

Also provided herein are methods for preparing compounds of Formula XXI,

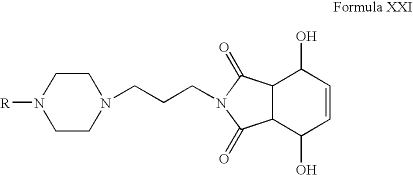

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises:
   hydrolyzing a compound of Formula XX

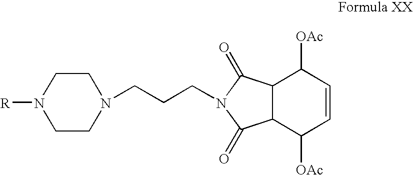

to form a compound of Formula XXI.

Also provided herein are methods for preparing compounds of Formula XXII,

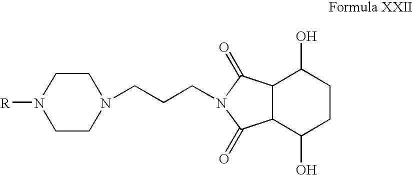

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises:
   reducing a compound of Formula XXI

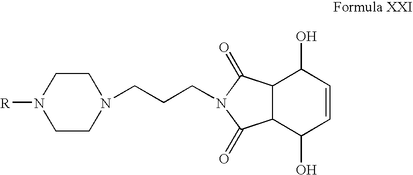

to form a compound of Formula XXII.

Also provided herein are methods for preparing compounds of Formula XXV,

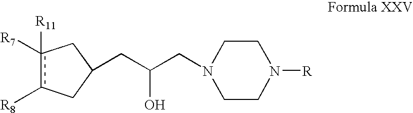

pharmaceutically acceptable salts, pharmaceutically acceptable solvates, enantiomers, diastereomers, N-oxides, prodrugs, polymorphs or metabolites thereof,
wherein R₇ and R₈ each can independently be hydrogen, alkyl, alkynyl, cycloalkyl, halogen, hydroxy, aryl, acetoxy, heterocycle,

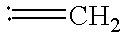

(wherein [symbol] can be the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

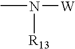

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

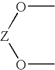

(wherein Z can be CO or SO), R₁₁ can be no atom hydrogen, alkyl, alkenyl, alkynyl, cycloalkyl, aryl, or heterocycle; and
R can be alkyl, alkenyl, alkynyl, cycloalkyl, aryl or heterocycle;
which method comprises:
   (a) reacting isoindole-1,3-dione with 2-chloromethyl oxirane to form 2-oxiranylmethyl-isoindole-1,3-dione
   (b) reacting 2-oxiranylmethyl-isoindole-1,3-dione with a compound of Formula VI

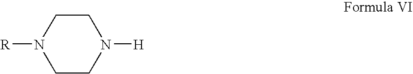

to form a compound of Formula XXIII,

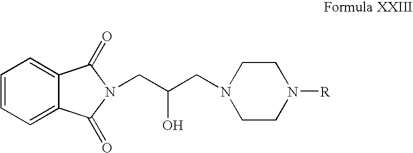

(c) reacting a compound of Formula XXIII with hydrazine hydrate to form a compound of Formula XXIV, and

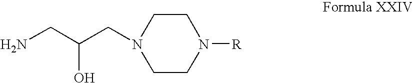

(d) reacting a compound of Formula XXIV with a compound of Formula XV

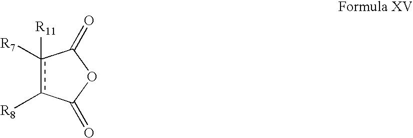

to form a compound of Formula XXV.

Also provided herein are methods for preparing compounds of Formula XXVII,

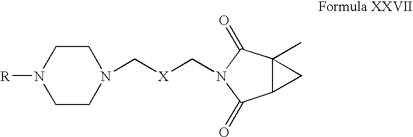

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle and X can be CO, CS or CHY (wherein Y can be hydrogen, hydroxy, halogen, alkoxy or haloalkoxy),
which method comprises:
   reacting a compound of Formula XXVI with a methylating agent

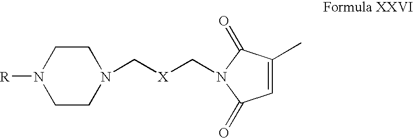

to form a compound of Formula XXVII.

Also provided herein are methods for preparing compounds of Formula XXIX,

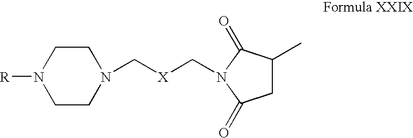

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle and X can be CO, CS or CHY (wherein Y can be hydrogen, hydroxy, halogen, alkoxy or haloalkoxy),
which method comprises:
   reducing a compound of Formula XXVI

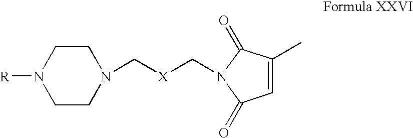

to form a compound of Formula XXIX.

Also provided herein are methods for preparing compounds of Formula XXX,

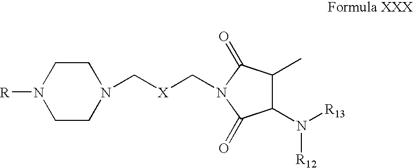

pharmaceutically acceptable salts, pharmaceutically acceptable solvates, enantiomers, diastereomers, N-oxides, prodrugs, polymorphs and metabolites thereof, wherein R can be alkyl, alkenyl, alkynyl, cycloalkyl, aryl or heterocycle; X can be CO, CS or CHY (wherein Y can be hydrogen, hydroxy, halogen, alkoxy or haloalkoxy); R₁₂ can be alkyl, alkenyl, alkynyl, cycloalkyl, cycloalkenyl, aryl or heterocycle; and R₁₃ can be hydrogen, alkyl, cycloalkyl, aryl or heterocycle;
which method comprises:
reacting a compound of Formula XXVI

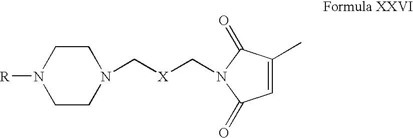

with a compound of Formula XXVIII

R₁₂NHR₁₃ Formula XXVIII
to form a compound of Formula XXX.

Also provided herein are methods for preparing compounds of Formula XXXIII,

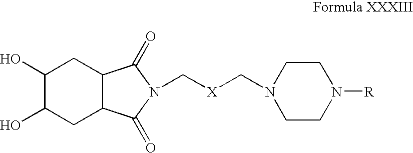

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle and X can be CO, CS or CHY (wherein Y can be hydrogen, hydroxy, halogen, alkoxy or haloalkoxy),
which method comprises:
   (a) reacting a compound of Formula XXXI

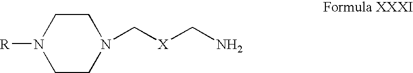

with tetrahydrophthalimide to form a compound of Formula XXXII, and

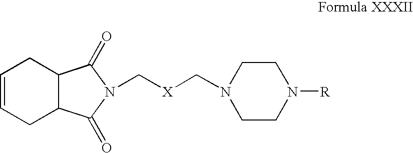

(b) oxidizing a compound of Formula XXXII to form a compound of Formula XXXIII:

Also provided herein are methods for preparing compounds of Formula XXXIV,

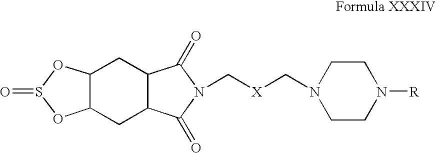

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle and X can be CO, CS or CHY (wherein Y can be hydrogen, hydroxy, halogen, alkoxy or haloalkoxy),
which method comprises:
   reacting a compound of Formula XXXIII

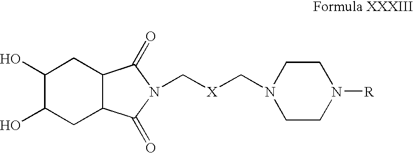

with diethyl amino sulfur trifluoride to form a compound of Formula XXXIV.

Also provided herein are methods for preparing compounds of Formula XXXV,

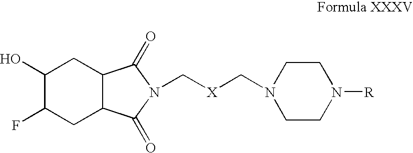

pharmaceutically acceptable salts, pharmaceutically acceptable solvates, enantiomers, diastereomers, N-oxides, prodrugs, polymorphs oir metabolites thereof, wherein R can be alkyl, alkenyl, alkynyl, cycloalkyl, aryl or heterocycle and X can be CO, CS or CHY (wherein Y can be hydrogen, hydroxy, halogen, alkoxy or haloalkoxy),
which method comprises:
   reacting a compound of Formula XXXIV

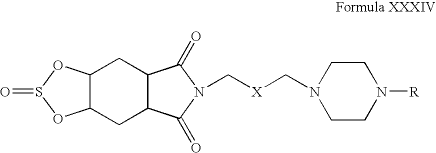

with diethyl amino sulfur trifluoride to form a compound of Formula XXXV.

Also provided herein are methods for preparing compounds of Formula XXXVI,

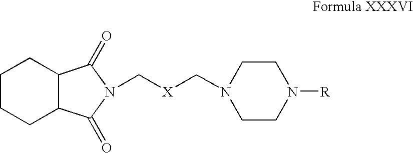

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle and X can be CO, CS or CHY (wherein Y can be hydrogen, hydroxy, halogen, alkoxy or haloalkoxy),
which method comprises:
   reducing a compound of Formula XXXII

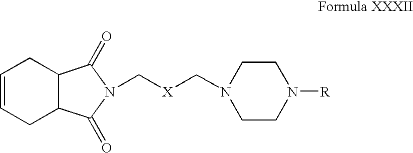

to form a compound of Formula XXXVI.

Also provided herein are methods for preparing compounds of Formula XL,

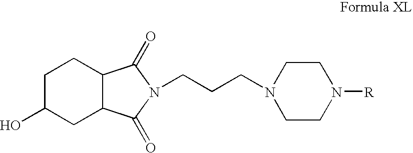

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises:
   (a) reacting a compound of Formula XXXVII

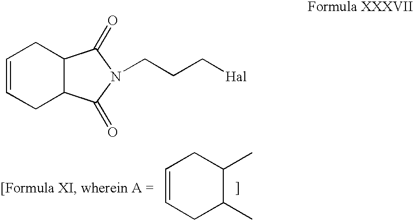

with a peroxy acid to form a compound of Formula XXXVIII,

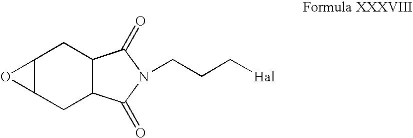

(b) reacting a compound of Formula XXXVIII with a compound of Formula VI

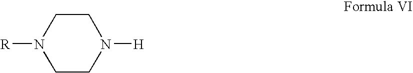

to form a compound of Formula XXXIX, and

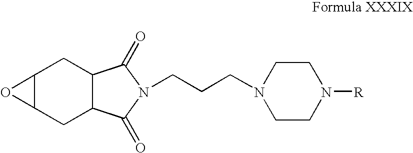

(c) reducing a compound of Formula XXXIX to form a compound of Formula XL.

Also provided herein are methods for preparing compounds of Formula XLI,

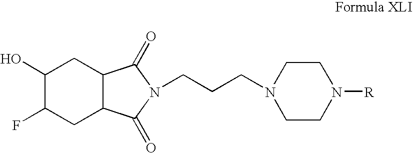

pharmaceutically acceptable salts, pharmaceutically acceptable solvates, enantiomers, diastereomers, N-oxides, prodrugs, polymorphs or metabolites thereof, wherein R can be alkyl, alkenyl, alkynyl, cycloalkyl, aryl or heterocycle,
which method comprises fluorinating a compound of Formula XXXIX

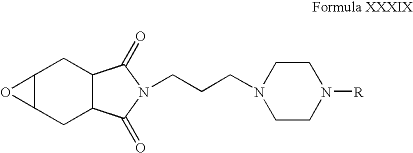

to form a compound of Formula XLI.

### DETAILED DESCRIPTION OF THE INVENTION

The present invention provides α₁ₐ and/or α_{1d} adrenergic receptor antagonists, which can be used for treatment of benign prostatic hyperplasia (BPH) or related symptoms thereof, or lower urinary tract symptoms (LUTS) with or without BPH. The present invention also provides for processes for the synthesis of such compounds. Also provided herein are pharmaceutically acceptable salts, pharmaceutically acceptable solvates, enantiomers, diastereomers, polymorphs or N-oxide of such compounds. Also provided are pharmaceutical compositions containing the disclosed compounds and one or more pharmaceutically acceptable carriers, excipients or diluents, which can be used for the treatment of BPH or related symptoms thereof or LUTS with or without BPH.

Provided herein are compounds having the structure of Formula I,

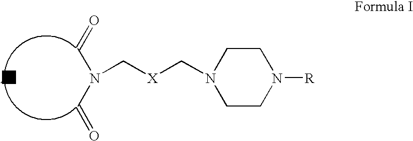

pharmaceutically acceptable salts, pharmaceutically acceptable solvates, enantiomers, diastereomers, N-oxides, prodrugs, polymorphs and metabolites thereof, wherein:
A can be,

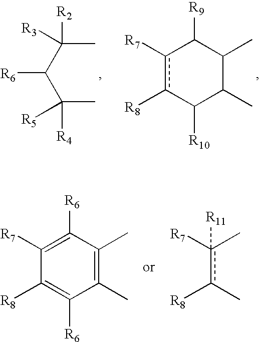

wherein, R₂, R₃, R₄ and R₅ can independently be hydrogen, alkyl or phenyl, R₆ is hydrogen, alkyl, phenyl, hydroxy or alkoxy, R₇ and R₈ can independently be hydrogen, alkyl, alkynyl, cycloalkyl, halogen, hydroxy, aryl, acetoxy, heterocycle,

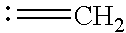

wherein [symbol] is the point of attachment) or R₁₂-Q-(CH₂)ₘ— (wherein m can be an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or

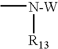

(wherein, W can be no atom, carbonyl, carboxylate or amide, R₁₃ can be hydrogen, alkyl, cycloalkyl, aryl or heterocycle), R₇ and R₈ together can be cycloalkyl, cycloalkenyl, bicyclic alkyl, bicyclic alkenyl, aryl, heterocycle or

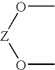

(wherein Z can be CO or SO), R₉ and R₁₀ can be independently hydrogen, hydroxy, alkoxy, acetyl, acetyloxy, R₁₁ can be no atom hydrogen, alkyl, alkenyl, alkynyl, cycloalkyl, aryl or heterocycle; X can be CO, CS or CHY (wherein Y can be hydrogen, hydroxy, halogen, alkoxy or haloalkoxy); R can be alkyl, alkenyl, alkynyl, cycloalkyl, aryl or heterocycle;
with the provisios that
   (a) when A is

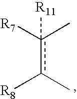

X is —CH₂— and R₁₁ is hydrogen then R₇ can be hydrogen or alkyl with the further provisio that when R₇ is alkyl and R₈ is R₁₂ NH—, then R₁₂ can be substituted alkyl wherein the substituents are selected from aryl or heterocyclyl
(b) when A is

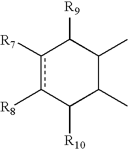

and X is —CH₂—, then none of R₇, R₈, R₉ R₁₀ are hydrogen or halogen,
(c) When A is

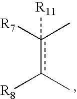

X is —CH₂—, and R₁₁ is no atom, then R₇ can be ═CH₂.

In one embodiment, there are provided compounds of Formula I, wherein:
A can be;

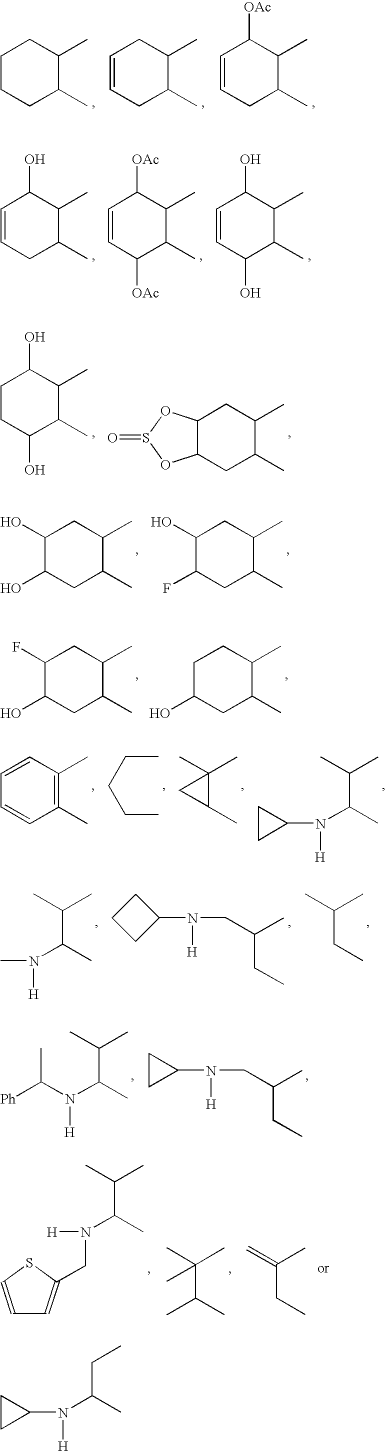

X can be CHOH, CO, CH₂ or CHF;
R can be 2-methoxy phenyl, 3-fluoro-2-methoxy phenyl, 5-fluoro-2-methoxy phenyl, 4-fluoro-2-methoxyphenyl, 2-methoxy-5-methyl phenyl, 2-n-propoxyphenyl, 5-fluoro2-n-propoxyphenyl, 2-ethoxy phenyl, 2-isopropoxy phenyl, 4-fluoro-2-isopropoxyphenyl, 4-nitro-2-isopropoxyphenyl, 3-fluoro-2-isopropoxy phenyl, 5-fluoro-2-isopropoxy phenyl, 2-cyclopentoxy-5-fluoro phenyl, 2-cyclopentoxy phenyl, O-tolyl, 2-trifluoroethoxy phenyl, 5-fluoro-2-trifluoromethoxy phenyl or 2-(2,2,3,3-tetrafluoropropoxy)phenyl.

In another aspect, there are provided compounds selected from:
2-{2-Hydroxy-3-[4-(2-isopropoxy-phenyl)-piperazin-1-yl]-propyl}-hexahydro-isoindole-1,3-dione (Compound No. 1),
2-{2-Hydroxy-3-[4-(2-isopropoxy-phenyl)-piperazin-1-yl]-propyl}-hexahydro-isoindole-1,3-dione hydrochloride salt (Compound No. 2),
2-{3-[4-(2-Isopropoxy-phenyl)-piperazin-1-yl]-2-oxo-propyl}-hexahydroisoindole-1,3-dione (Compound No. 3),
2-{3-[4-(2-Isopropoxy-phenyl)-piperazin-1-yl]-2-oxo-propyl}-hexahydroisoindole-1,3-dione hydrochloride salt (Compound No. 4),
2-{3-[4-(2-Isopropoxy-phenyl)-piperazin-1-yl]-2-oxo-propyl}-3a,4,7,7a-tetrahydro-isoindole-1,3-dione (Compound No. 5),
2-{3-[4-(2-Isopropoxy-phenyl)-piperazin-1-yl]-2-oxo-propyl}-3a,4,7,7a-tetrahydro-isoindole-1,3-dione hydrochloride salt (Compound No. 6),
2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione (Compound No. 7),
2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione hydrochloride salt (Compound No. 8),
2-(2-Hydroxy-3-{4-[2-(2,2,3,3-tetrafluoro-propoxy)-phenyl]-piperazin-1-yl}-propyl)-3a,4,7,7a-tetrahydro-isoindole-1,3-dione (Compound No. 9),
2-(2-Hydroxy-3-{4-[2-(2,2,3,3-tetrafluoro-propoxy)-phenyl]-piperazin-1-yl}-propyl)-3a,4,7,7a-tetrahydro-isoindole-1,3-dione hydrochloride salt (Compound No. 10),
2-{2-Hydroxy-3-[4-(2-isopropoxy-4-nitro-phenyl)-piperazin-1-yl]-propyl}-3a,4,7,7a-tetrahydro-isoindole-1,3-dione (Compound No. 11),
2-{2-Hydroxy-3-[4-(2-isopropoxy-4-nitro-phenyl)-piperazin-1-yl]-propyl}-3a,4,7,7a-tetrahydro-isoindole-1,3-dione hydrochloride salt (Compound No. 12),
2-{2-Fluoro-3-[4-(2-isopropoxy-phenyl)-piperazin-1-yl]-propyl}-3a,4,7,7a-tetrahydro-isoindole-1,3-dione (Compound No. 13),
2-{2-Fluoro-3-[4-(2-isopropoxy-phenyl)-piperazin-1-yl]-propyl}-3a,4,7,7a-tetrahydro-isoindole-1,3-dione hydrochloride salt (Compound No. 14),
Acetic acid 2-{3-[4-(2-methoxy-phenyl)-piperazin-1-yl]-propyl}-1,3-dioxo-2,3,3a,4,7,7a-hexahydro-1H-inden-4-yl ester (Compound No. 15),
Acetic acid 2-{3-[4-(2-methoxy-phenyl)-piperazin-1-yl]-propyl}-1,3-dioxo-2,3,3a,4,7,7a-hexahydro-1H-inden-4-yl ester hydrochloride salt (Compound No. 16),
4-Hydroxy-2-{3-[4-(2-methoxy-phenyl)-piperazin-1-yl]-propyl}-3a,4,7,7a-tetrahydro-isoindole-1,3-dione (Compound No. 17),
4-Hydroxy-2-{3-[4-(2-methoxy-phenyl)-piperazin-1-yl]-propyl}-3a,4,7,7a-tetrahydro-isoindole-1,3-dione hydrochloride salt (Compound No. 18),
2-{3-[4-(2-Methoxy-phenyl)-piperazin-1-yl}-2-oxo-propyl}-3a,4,7,7a-tetrahydro-isoindole-1,3-dione (Compound No. 19),
2-{3-[4-(2-Methoxy-phenyl)-piperazin-1-yl}-2-oxo-propyl}-3a,4,7,7a-tetrahydro-isoindole-1,3-dione hydrochloride salt (Compound No. 20),
2-{3-[4-(2-Cyclopentyloxy-phenyl)-piperazin-1-yl]-2-oxo-propyl}-3a,4,7,7a-tetrahydro-isoindole-1,3-dione (Compound No. 21),
2-{2-Oxo-3-[4-(2-propoxy-phenyl)-piperazin-1-yl]-propyl}-3a,4,7,7a-tetrahydro-isoindole-1,3-dione (Compound No. 22),
2-{2-Oxo-3-[4-(2-propoxy-phenyl)-piperazin-1-yl]-propyl}-3a,4,7,7a-tetrahydro-isoindole-1,3-dione hydrochloride salt (Compound No. 23),
2-{3-[4-(4-Fluoro-2-isopropoxy-phenyl)-piperazin-1-yl]-2-oxo-propyl}-3a,4,7,7a-tetrahydro-isoindole-1,3-dione (Compound No. 24),
2-{3-[4-(4-Fluoro-2-isopropoxy-phenyl)-piperazin-1-yl]-2-oxo-propyl}-3a,4,7,7a-tetrahydro-isoindole-1,3-dione hydrochloride salt (Compound No. 25),
2-{3-[4-(2-Isopropoxy-phenyl)-piperazin-1-yl]-2-oxo-propyl}-isoindole-1,3-dione (Compound No. 26),
2-{3-[4-(2-Isopropoxy-phenyl)-piperazin-1-yl]-2-oxo-propyl}-isoindole-1,3-dione hydrochloride salt (Compound No. 27),
2-(2-Oxo-3-{4-[2-(2,2,2-trifluoro-ethoxy)-phenyl]-piperazin-1-yl}-propyl)-3a,4,7,7a-tetrahydro-isoindole-1,3-dione (Compound No. 28),
2-(2-Oxo-3-{4-[2-(2,2,2-trifluoro-ethoxy)-phenyl]-piperazin-1-yl}-propyl)-3a,4,7,7a-tetrahydro-isoindole-1,3-dione hydrochloride salt (Compound No. 29),
6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione (Compound No. 30),
6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione hydrochloride salt (Compound No. 31),
1-[2-Oxo-3-(4-phenyl-piperazin-1-yl)-propyl]-pyrrolidine-2,5-dione (Compound No. 32),
1-{3-[4-{4-Fluoro-phenyl}-piperazin-1-yl]-2-oxo-propyl}-3-phenyl-piperidine-2,6-dione (Compound No. 33),
3,4-Dimethyl-1-{2-oxo-3-[4-(2-trifluoromethyl-phenyl)-piperazin-1-yl]-propyl}-2,5-dione (Compound No. 34),
1-{2-Fluoro-3-[4-(4-fluorophenyl)piperazin-1-yl]-propyl}-piperidine-2,6-dione (Compound No. 35),
1-(2-Fluoro-3-{4-[2-(2,2,2-trifluoro-ethoxy)-phenyl]-piperazin-1-yl}propyl)-3,4-dimethylpyrrole-2,5-dione (Compound No. 36),
1-{3-[4-(2-Cyclopentyloxy-phenyl)-piperazin-1-yl]-2-hydroxy-propyl}-3-cyclopropylamino-4-methyl-pyrrolidine-2,5-dione (Compound No. 37),
1-{3-[4-(2-Cyclopentyloxy-phenyl)-piperazin-1-yl]-2-hydroxy-propyl}-3-cyclopropylamino-4-methyl-pyrrolidine-2,5-dione hydrochloride salt (Compound No. 38),
1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione (Compound No. 39),
1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione hydrochloride salt (Compound No. 40),
1-{3-[4-(2-Cyclopentyloxy-5-fluoro-phenyl)-piperazin-1-yl]-2-hydroxy-propyl}-3-cyclopropylaminomethyl-pyrrolidine-2,5-dione (Compound No. 41),
1-{3-[4-(2-Cyclopentyloxy-5-fluoro-phenyl)-piperazin-1-yl]-2-hydroxy-propyl}-3-cyclopropylaminomethyl-pyrrolidine-2,5-dione hydrochloride salt (Compound No. 42),
2-{3-[4-(5-Fluoro-2-isopropoxy-phenyl)-piperazin-1-yl]-propyl}-5,6-dihydroxy-hexahydro-isoindole-1,3-dione (Compound No. 43),
2-{3-[4-(5-Fluoro-2-isopropoxy-phenyl)-piperazin-1-yl]-propyl}-5,6-dihydroxy-hexahydro-isoindole-1,3-dione hydrochloride salt (Compound No. 44),
1-{3-[4-(2-Cyclopentyloxy-5-fluoro-phenyl)-piperazin-1-yl]-2-hydroxy-propyl}-3-methyl-4-methylamino-pyrrolidine-2,5-dione (Compound No. 45),
1-{3-[4-(2-Cyclopentyloxy-5-fluoro-phenyl)-piperazin-1-yl]-2-hydroxy-propyl}-3-methyl-4-methylamino-pyrrolidine-2,5-dione hydrochloride salt (Compound No. 46),
1-{3-[4-(2-Methoxy-5-methyl-phenyl)-piperazin-1-yl]-propyl}-piperidine-2,6-dione (Compound No. 47),
1-{3-[4-(2-Methoxy-5-methyl-phenyl)-piperazin-1-yl]-propyl}-piperidine-2,6-dione hydrochloride salt (Compound No. 48),
5,6-Dihydroxy-2-{3-[4-(2-methoxy-5-methyl-phenyl)-piperazin-1-yl]-propyl}-hexahydro-isoindole-1,3-dione (Compound No. 49),
5,6-Dihydroxy-2-{3-[4-(2-methoxy-5-methyl-phenyl)-piperazin-1-yl]-propyl}-hexahydro-isoindole-1,3-dione hydrochloride salt (Compound No. 50),
1-(3-{4-[5-Fluoro-2-(2,2,2-trifluoro-ethoxy)-phenyl]-piperazin-1-yl}-propyl)-piperidine-2,6-dione (Compound No. 51),
1-(3-{4-[5-Fluoro-2-(2,2,2-trifluoro-ethoxy)-phenyl]-piperazin-1-yl}-propyl)-piperidine-2,6-dione hydrochloride salt (Compound No. 52),
2-{3-[4-(2-Cyclopentyloxy-phenyl)-piperazin-1-yl]-propyl}-5,6-dihydroxy-hexahydro-isoindole-1,3-dione (Compound No. 53),
2-{3-[4-(2-Cyclopentyloxy-phenyl)-piperazin-1-yl]-propyl}-5,6-dihydroxy-hexahydro-isoindole-1,3-dione hydrochloride salt (Compound No. 54),
3-{3-[4-(3-Fluoro-2-methoxy-phenyl)-piperazin-1-yl]-propyl)-1-methyl-3-aza-bicyclo[3.1.0]hexane-2,4-dione (Compound No. 55),
3-{3-[4-(3-Fluoro-2-methoxy-phenyl)-piperazin-1-yl]-propyl)-1-methyl-3-aza-bicyclo[3.1.0]hexane-2,4-dione hydrochloride salt (Compound No. 56),
3-{3-[4-(5-Fluoro-2-methoxy-phenyl)-piperazin-1-yl]-propyl}-1-methyl-3-aza-bicyclo[3.1.0]hexane-2,4-dione (Compound No. 57),
3-{3-[4-(5-Fluoro-2-methoxy-phenyl)-piperazin-1-yl]-propyl}-1-methyl-3-aza-bicyclo[3.1.0]hexane-2,4-dione hydrochloride salt (Compound No. 58),
3-{3-[4-(2-Cyclopentyloxy-phenyl)-piperazin-1-yl]-propyl}-1-methyl-3-aza-bicyclo[3.1.0]hexane-2,4-dione (Compound No. 59),
3-{3-[4-(2-Cyclopentyloxy-phenyl)-piperazin-1-yl]-propyl}-1-methyl-3-aza-bicyclo[[3.1.0]hexane-2,4-dione hydrochloride salt (Compound No. 60),
5-Fluoro-6-hydroxy-2-{2-hydroxy-3-[4-(2-isopropoxy-phenyl)-piperazin-1-yl]-propyl}-hexahydro-isoindole-1,3-dione (Compound No. 61),
5-Fluoro-6-hydroxy-2-{2-hydroxy-3-[4-(2-isopropoxy-phenyl)-piperazin-1-yl]-propyl}-hexahydro-isoindole-1,3-dione hydrochloride salt (Compound No. 62),
3-{3-[4-(5-Fluoro-2-isopropoxy-phenyl)-piperazin-1-yl]-propyl}-1-methyl-3-aza-bicyclo[3.1.0]hexane-2,4-dione (Compound No. 63),
3-{3-[4-(5-Fluoro-2-isopropoxy-phenyl)-piperazin-1-yl]-propyl}-1-methyl-3-aza-bicyclo[3.1.0]hexane-2,4-dione hydrochloride salt (Compound No. 64),
5-Fluoro-6-hydroxy-2-{3-[4-(2-isopropoxy-phenyl)-piperazin-1-yl]-propyl}-hexahydro-isoindole-1,3-dione (Compound No. 65),
5-Fluoro-6-hydroxy-2-{3-[4-(2-isopropoxy-phenyl)-piperazin-1-yl]-propyl}-hexahydro-isoindole-1,3-dione hydrochloride salt (Compound No. 66),
2-{3-[4-(2-Cyclopentyloxy-phenyl)-piperazin-1-yl]-2-hydroxy-propyl}-hexahydro-isoindole-1,3-dione (Compound No. 67),
2-{3-[4-(2-Cyclopentyloxy-phenyl)-piperazin-1-yl]-2-hydroxy-propyl}-hexahydro-isoindole-1,3-dione hydrochloride salt (Compound No. 68),
5-Hydroxy-2-{3-[4-(2-methoxy-phenyl)-piperazin-1-yl]-propyl}-hexahydro-isoindole-1,3-dione (Compound No. 69),
5-Hydroxy-2-{3-[4-(2-methoxy-phenyl)-piperazin-1-yl]-propyl}-hexahydro-isoindole-1,3-dione hydrochloride salt (Compound No. 70),
2-{3-[4-(2-Cyclopentyloxy-5-fluoro-phenyl)-piperazin-1-yl]-propyl}-5-hydroxy-hexahydro-isoindole-1,3-dione (Compound No. 71),
2-{3-[4-(2-Cyclopentyloxy-5-fluoro-phenyl)-piperazin-1-yl]-propyl}-5-hydroxy-hexahydro-isoindole-1,3-dione hydrochloride salt (Compound No. 72),
2-{3-[4-(2-Cyclopentyloxy-phenyl)-piperazin-1-yl]-propyl}-5-hydroxy-hexahydro-isoindole-1,3-dione (Compound No. 73),
2-{3-[4-(2-Cyclopentyloxy-phenyl)-piperazin-1-yl]-propyl}-5-hydroxy-hexahydro-isoindole-1,3-dione hydrochloride salt (Compound No. 74),
2-{3-[4-(2-Cyclopentyloxy-phenyl)-piperazin-1-yl]-2-oxo-propyl}-5,6-dihydroxy-hexahydro-isoindole-1,3-dione (Compound No. 75),
2-{3-[4-(2-Cyclopentyloxy-phenyl)-piperazin-1-yl]-2-oxo-propyl}-5,6-dihydroxy-hexahydro-isoindole-1,3-dione hydrochloride salt (Compound No. 76),
2-{3-[4-(2-Cyclopentyloxy-5-fluoro-phenyl)-piperazin-1-yl]-2-oxo-propyl}-3a,4,7,7a-tetrahydro-isoindole-1,3-dione (Compound No. 77),
2-{3-[4-(2-Cyclopentyloxy-5-fluoro-phenyl)-piperazin-1-yl]-2-oxo-propyl}-3a,4,7,7a-tetrahydro-isoindole-1,3-dione hydrochloride salt (Compound No. 78),
2-{3-[4-(2-Cyclopentyloxy-5-fluoro-phenyl)-piperazin-1-yl]-2-hydroxy-propyl}-hexahydro-isoindole-1,3-dione (Compound No. 79),
2-{3-[4-(2-Cyclopentyloxy-5-fluoro-phenyl)-piperazin-1-yl]-2-hydroxy-propyl}-hexahydro-isoindole-1,3-dione hydrochloride salt (Compound No. 80),
5-Fluoro-2-{3-[4-(5-fluoro-2-methoxy-phenyl)-piperazin-1-yl]-propyl}-6-hydroxy-hexahydro-isoindole-1,3-dione (Compound No. 81),
5-Fluoro-2-{3-[4-(5-fluoro-2-methoxy-phenyl)-piperazin-1-yl]-propyl}-6-hydroxy-hexahydro-isoindole-1,3-dione hydrochloride salt (Compound No. 82),
2-{3-[4-(5-Fluoro-2-isopropoxy-phenyl)-piperazin-1-yl]-propyl}-5-hydroxy-hexahydro-isoindole-1,3-dione (Compound No. 83),
2-{3-[4-(5-Fluoro-2-isopropoxy-phenyl)-piperazin-1-yl]-propyl}-5-hydroxy-hexahydro-isoindole-1,3-dione hydrochloride salt (Compound No. 84),
5-Fluoro-2-{3-[4-(5-fluoro-2-isopropoxy-phenyl)-piperazin-1-yl]-propyl}-hexahydro-isoindole-1,3-dione (Compound No. 85),
5-Fluoro-2-{3-[4-(5-fluoro-2-isopropoxy-phenyl)-piperazin-1-yl]-propyl}-6-hydroxy-hexahydro-isoindole-1,3-dione hydrochloride salt (Compound No. 86),
1-{3-[4-(5-Fluoro-2-isopropoxy-phenyl)-piperazin-1-yl]-2-hydroxy-propyl}-piperidine-2,6-dione (Compound No. 87),
1-{3-[4-(5-Fluoro-2-isopropoxy-phenyl)-piperazin-1-yl]-2-hydroxy-propyl}-piperidine-2,6-dione hydrochloride salt (Compound No. 88),
1-{3-[4-(2-Cyclopentyloxy-5-fluoro-phenyl)-piperazin-1-yl]-2-hydroxy-propyl}-piperidine-2,6-dione (Compound No. 89),
1-{3-[4-(2-Cyclopentyloxy-5-fluoro-phenyl)-piperazin-l -yl]-2-hydroxy-propyl}-piperidine-2,6-dione hydrochloride salt (Compound No. 90),
Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester (Compound No. 91),
Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester hydrochloride salt (Compound No. 92),
Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester (Compound No. 93),
Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester hydrochloride salt (Compound No. 94),
2-{3-[4-(5-Fluoro-2-isopropoxy-phenyl)-piperazin-1-yl]-propyl}-4,7-dihydroxy-3a,4,7,7a-tetrahydro-isoindole-1,3-dione (Compound No. 95),
2-{3-[4-(5-Fluoro-2-isopropoxy-phenyl)-piperazin-1-yl]-propyl}-4,7-dihydroxy-3a,4,7,7a-tetrahydro-isoindole-1,3-dione hydrochloride salt (Compound No. 96),
1-{3-[4-(5-Fluoro-2-propoxy-phenyl)-piperazin-1-yl]-propyl}-piperidine-2,6-dione (Compound No. 97),
1-(3-[4-(5-Fluoro-2-propoxy-phenyl)-piperazin-1-yl]-propyl}-piperidine-2,6-dione hydrochloride salt (Compound No. 98),
1-{3-[4-(5-Fluoro-2-propoxy-phenyl)-piperazin-1-yl]-2-hydroxy-propyl)-piperidine-2,6-dione (Compound No. 99),
1-{3-[4-(5-Fluoro-2-propoxy-phenyl)-piperazin-1-yl]-2-hydroxy-propyl)-piperidine-2,6-dione hydrochloride salt (Compound No. 100),
2-{3-[4-(2-Cyclopentyloxy-5-fluoro-phenyl)-piperazin-1-yl]-propyl}-5,6-dihydroxy-hexahydro-isoindole-1,3-dione (Compound No. 101),
2-{3-[4-(2-Cyclopentyloxy-5-fluoro-phenyl)-piperazin-1-yl]-propyl}-5,6-dihydroxy-hexahydro-isoindole-1,3-dione hydrochloride salt (Compound No. 102),
2-{3-[4-(5-Fluoro-2-propoxy-phenyl)-piperazin-1-yl]-propyl}-5,6-dihydroxy-hexahydro-isoindole-1,3-dione (Compound No. 103),
2-{3-[4-(5-Fluoro-2-propoxy-phenyl)-piperazin-1-yl]-propyl}-5,6-dihydroxy-hexahydro-isoindole-1,3-dione hydrochloride salt (Compound No. 104),
2-{3-[4-(2-Cyclopentyloxy-phenyl)-piperazin-1-yl]-propyl}-5-fluoro-6-hydroxy-hexahydro-isoindole-1,3-dione (Compound No. 105),
2-{3-[4-(2-Cyclopentyloxy-phenyl)-piperazin-1-yl]-propyl}-5-fluoro-6-hydroxy-hexahydro-isoindole-1,3-dione hydrochloride salt (Compound No. 106),
2-{3-[4-(2-Cyclopentyloxy-5-fluoro-phenyl)-piperazin-1-yl]-propyl}-5-fluoro-6-hydroxy-hexahydro-isoindole-1,3-dione (Compound No. 107),
2-{3-[4-(2-Cyclopentyloxy-5-fluoro-phenyl)-piperazin-1-yl]-propyl}-5-fluoro-6-hydroxy-hexahydro-isoindole-1,3-dione hydrochloride salt (Compound No. 108),
3-Cyclopropylaminomethyl-1-{2-hydroxy-3-[4-(2-methoxy-phenyl)-piperazin-1-yl]-propyl}-pyrrolidine-2,5-dione (Compound No. 109),
3-Cyclopropylaminomethyl-1-{2-hydroxy-3-[4-(2-methoxy-phenyl)-piperazin-1-yl]-propyl}-pyrrolidine-2,5-dione hydrochloride salt (Compound No.110),
3-Cyclopropylaminomethyl-1-{2-hydroxy-3-[4-(2-methoxy-phenyl)-piperazin-1-yl]-propyl}-4-methyl-pyrrolidine-2,5-dione(Compound No. 111),
3-Cyclopropylaminomethyl-1-{2-hydroxy-3-[4-(2-methoxy-phenyl)-piperazin-1-yl]-propyl}-4-methyl-pyrrolidine-2,5-dione hydrochloride salt (Compound No. 112),
3-Cyclobutylaminomethyl-1-{2-hydroxy-3-[4-(2-methoxy-phenyl)-piperazin-1-yl]-propyl}-pyrrolidine-2,5-dione (Compound No. 113),
3-Cyclobutylaminomethyl-1-{2-hydroxy-3-[4-(2-methoxy-phenyl)-piperazin-1-yl]-propyl}-pyrrolidine-2,5-dione hydrochloride salt (Compound No. 114),
1-{2-Hydroxy-3-[4-(2-methoxy-phenyl)-piperazin-1-yl]-propyl}-3-methyl-pyrrolidine-2,5-dione (Compound No. 115),
1-{2-Hydroxy-3-[4-(2-methoxy-phenyl)-piperazin-l -yl]-propyl}-3-methyl-pyrrolidine-2,5-dione hydrochloride salt (Compound No. 116),
2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione (Compound No. 117),
2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione hydrochloride salt (Compound No. 118),
2-{3-[4-(2-Cyclopentyloxy-5-fluoro-phenyl)-piperazin-1-yl]-2-oxo-propyl}-5,6-dihydroxy-hexahydro-isoindole-1,3-dione (Compound No. 119),
2-{3-[4-(2-Cyclopentyloxy-5-fluoro-phenyl)-piperazin-1-yl]-2-oxo-propyl}-5,6-dihydroxy-hexahydro-isoindole-1,3-dione hydrochloride salt (Compound No. 120),
2-{3-[4-(5-Fluoro-2-isopropoxy-phenyl)-piperazin-1-yl]-2-oxo-propyl}-5,6-dihydroxy-hexahydro-isoindole-1,3-dione (Compound No. 121),
2-{3-[4-(5-Fluoro-2-isopropoxy-phenyl)-piperazin-1-yl]-2-oxo-propyl}-5,6-dihydroxy-hexahydro-isoindole-1,3-dione hydrochloride salt (Compound No. 122),
2-{3-[4-(5-Fluoro-2-isopropoxy-phenyl)-piperazin-1-yl]-2-hydroxy-propyl}-5,6-dihydroxy-hexahydro-isoindole-1,3-dione (Compound No. 123),
2-{3-[4-(5-Fluoro-2-isopropoxy-phenyl)-piperazin-1-yl]-2-hydroxy-propyl}-5,6-dihydroxy-hexahydro-isoindole-1,3-dione hydrochloride salt (Compound No. 124),
2-{3-[4-(2-Cyclopentyloxy-5-fluoro-phenyl)-piperazin-1-yl]-2-hydroxy-propyl}-5,6-dihydroxy-hexahydro-isoindole-1,3-dione (Compound No. 125),
2-{3-[4-(2-Cyclopentyloxy-5-fluoro-phenyl)-piperazin-1-yl]-2-hydroxy-propyl}-5,6-dihydroxy-hexahydro-isoindole-1,3-dione hydrochloride salt (Compound No. 126),
1-{3-[4-(2-Cyclopentyloxy-5-fluoro-phenyl)-piperazin-1-yl]-propyl}-piperidine-2,6-dione (Compound No. 127),
1-{3-[4-(2-Cyclopentyloxy-5-fluoro-phenyl)-piperazin-1-yl]-propyl}-piperidine-2,6-dione hydrochloride salt (Compound No. 128),
1-{3-[4-(3-Fluoro-2-isopropoxy-phenyl)-piperazin-1-yl]-propyl}-piperidine-2,6-dione (Compound No. 129),
1-{3-[4-(3-Fluoro-2-isopropoxy-phenyl)-piperazin-1-yl]-propyl}-piperidine-2,6-dione hydrochloride salt (Compound No. 130),
1-{3-[4-(3-Fluoro-2-methoxy-phenyl)-piperazin-1-yl]-propyl}-piperidine-2,6-dione (Compound No. 131),
1-{3-[4-(3-Fluoro-2-methoxy-phenyl)-piperazin-1-yl]-propyl}-piperidine-2,6-dione hydrochloride salt (Compound No. 132),
1-{3-[4-(3-Fluoro-2-isopropoxy-phenyl)-piperazin-1-yl]-propyl}-3-methylene-pyrrolidine-2,5-dione (Compound No. 133),
1-{3-[4-(3-Fluoro-2-isopropoxy-phenyl)-piperazin-1-yl]-propyl}-3-methylene-pyrrolidine-2,5-dione hydrochloride salt (Compound No. 134),
1-{3-[4-(5-Fluoro-2-isopropoxy-phenyl)-piperazin-1-yl]-propyl}-3-methylene-pyrrolidine-2,5-dione (Compound No. 135),
1-{3-[4-(5-Fluoro-2-isopropoxy-phenyl)-piperazin-1-yl]-propyl}-3-methylene-pyrrolidine-2,5-dione hydrochloride salt (Compound No. 136),
1-{3-[4-(5-Fluoro-2-(2,2,3,3-tetrafluoro-propoxy)-phenyl]-piperazin-1-yl}-propyl)-piperidine-2,6-dione (Compound No. 137),
1-{3-[4-(5-Fluoro-2-(2,2,3,3-tetrafluoro-propoxy)-phenyl]-piperazin-1-yl}-propyl)-piperidine-2,6-dione hydrochloride salt (Compound No. 138),
1-{3-[4-(2-Methoxy-phenyl)-piperazin-1-yl]-propyl)-3-methyl-4-(1-phenyl-ethylamino)-pyrrolidine-2,5-dione (Compound No. 139),
1-{3-[4-(2-Methoxy-phenyl)-piperazin-1-yl]-propyl)-3-methyl-4-(1-phenyl-ethylamino)-pyrrolidine-2,5-dione hydrochloride salt (Compound No. 140),
1-{3-[4-(5-Fluoro-2-trifluoromethoxy-phenyl)-piperazin-1-yl]-propyl)-piperidine-2,6-dione (Compound No. 141),
1-{3-[4-(5-Fluoro-2-trifluoromethoxy-phenyl)-piperazin-1-yl]-propyl)-piperidine-2,6-dione hydrochloride salt (Compound No. 142),
Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-yl ester (Compound No. 143),
Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-yl ester hydrochloride salt (Compound No. 144),
2-{3-[4-(2-Ethoxy-phenyl)-piperazin-1-yl]-propyl)-4,7-dihydroxy-3a,4,7,7a-tetrahydro-isoindole-1,3-dione (Compound No. 145),

02-{3-[4-(2-Ethoxy-phenyl)-piperazin-1-yl]-propyl)-4,7-dihydroxy-3a,4,7,7a-tetrahydro-isoindole-1,3-dione hydrochloride salt (Compound No. 146),
3-Cyclopropylamino-1-{3-[4-(2-ethoxy-phenyl)-piperazin-1-yl]-propyl}-pyrrolidine-2,5-dione (Compound No. 147),
3-Cyclopropylamino-1-{3-[4-(2-ethoxy-phenyl)-piperazin-1-yl]-propyl}-pyrrolidine-2,5-dione hydrochloride salt (Compound No. 148),
Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester (Compound No. 149),
Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester hydrochloride salt (Compound No. 150),
1-{3-[4-(2-Cyclopentyloxy-phenyl)-piperazin-1-yl]-propyl)-3-cyclopropylamino-pyrrolidine-2,5-dione (Compound No. 151),
1-{3-[4-(2-Cyclopentyloxy-phenyl)-piperazin-1-yl]-propyl)-3-cyclopropylamino-pyrrolidine-2,5-dione hydrochloride salt (Compound No. 152),
4,7-Dihydroxy-2-{3-[4-(2-methoxy-phenyl)-piperazin-1-yl]-propyl)-3a,4,7,7a-tetrahydro-isoindole-1,3-dione (Compound No. 153),
4,7-Dihydroxy-2-{3-[4-(2-methoxy-phenyl)-piperazin-l -yl]-propyl)-3a,4,7,7a-tetrahydro-isoindole-1,3-dione hydrochloride salt (Compound No. 154),
Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester (Compound No. 155),
Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester hydrochloride salt (Compound No. 156),
2-{3-[4-(2-Cyclopentyloxy-phenyl)-piperazin-1-yl]-propyl)-4,7-dihydroxy-hexahydro-isoindole-1,3-dione (Compound No. 157),
2-{3-[4-(2-Cyclopentyloxy-phenyl)-piperazin-1-yl]-propyl)-4,7-dihydroxy-hexahydro-isoindole-1,3-dione hydrochloride salt (Compound No. 158),
2-{3-[4-(2-Cyclopentyloxy-phenyl)-piperazin-1-yl]-propyl}-4,7-dihydroxy-3a,4,7,7a-tetrahydro-isoindole-1,3-dione (Compound No. 159),
2-{3-[4-(2-Cyclopentyloxy-phenyl)-piperazin-1-yl]-propyl}-4,7-dihydroxy-3a,4,7,7a-tetrahydro-isoindole-1,3-dione hydrochloride salt (Compound No. 160),
3-Methylene-1-[3-(4-O-tolyl-piperazin-1-yl)-propyl]-pyrrolidine-2,5-dione (Compound No. 161),
3-Methylene-1-[3-(4-O-tolyl-piperazin-1-yl)-propyl]-pyrrolidine-2,5-dione hydrochloride salt (Compound No. 162),
1-{3-[4-(2-Methoxy-phenyl)-piperazin-1-yl]-propyl)-3-methyl-4-[(thiophen-2-ylmethyl)-amino]-pyrrolidine-2,5-dione (Compound No. 163),
1-{3-[4-(2-Methoxy-phenyl)-piperazin-1-yl]-propyl)-3-methyl-4-[(thiophen-2-ylmethyl)-amino]-pyrrolidine-2,5-dione hydrochloride salt (Compound No. 164),
1-{3-[4-(2-Cyclopentyloxy-phenyl)-piperazin-1-yl]-propyl}-3-methylene-pyrrolidine-2,5-dione (Compound No. 165),
1-{3-[4-(2-Cyclopentyloxy-phenyl)-piperazin-1-yl]-propyl}-3-methylene-pyrrolidine-2,5-dione hydrochloride salt (Compound No. 166),
1-{3-[4-(5-Fluoro-2-methoxy-phenyl)-piperazin-1-yl]-propyl}-3,3,4-trimethyl-pyrrolidine-2,5-dione (Compound No. 167),
1-{3-[4-(5-Fluoro-2-methoxy-phenyl)-piperazin-1-yl]-propyl}-3,3,4-trimethyl-pyrrolidine-2,5-dione hydrochloride salt (Compound No. 168),
1-{3-[4-(2-Cyclopentyloxy-phenyl)-piperazin-1-yl]-propyl}-piperidine-2,6-dione (Compound No. 169),
1-{3-[4-(2-Cyclopentyloxy-phenyl)-piperazin-1-yl]-propyl}-piperidine-2,6-dione hydrochloride salt (Compound No. 170),
or their pharmaceutically acceptable salts, pharmaceutically acceptable solvates, enantiomers, diastereomers, N-oxides, prodrugs, polymorphs or metabolites.

In another aspect, provided are methods for treating a disease or disorder mediated through α₁ₐ and/or α_{1d} adrenergic receptors comprising administering to a patient in need thereof a therapeutically effective amount of a compound or pharmaceutical composition disclosed herein.

In yet another aspect, provided are methods for treating benign prostatic hyperplasia (BPH) or related symptoms comprising administering to a patient in need thereof a therapeutically effective amount of a compound or pharmaceutical composition disclosed herein.

In another aspect, provided are methods for treating lower urinary tract symptoms (LUTS) with or without BPH comprising administering to a patient in need thereof a therapeutically effective amount of a compound or pharmaceutical composition disclosed herein. LUTS may include, for example, irritative symptoms (e.g., frequent urination, urgent urination, nocturia and unstable bladder contractions), obstructive symptoms (e.g., hesitancy, poor stream, prolong urination, and feelings of incomplete emptying).

In another aspect, provided are methods for treating BPH or LUTS with or without BPH comprising administering to a patient in need thereof a therapeutically effective amount of one or more compounds (or compositions) described herein in combination with one or more bladder selective muscarinic receptor antagonists and/or testosterone 5α-reductase inhibitors.

In yet another aspect, provided are processes for preparing compounds disclosed herein.

The compounds of the present invention are potent adrenergic receptor antagonists. Such compounds exhibit low nanomolar affinity towards α₁ₐ and α_{1d} adrenoceptor subtypes and good selectivity for α₁ₐ vs. α_{1b} adrenoceptor subtypes. α₁ₐ adrenoceptors are involved in relieving the obstructive symptoms, whereas α_{1d} adrenoceptor antagonism is associated in alleviation of irritative symptoms. The relatively lower affinity to α_{1b} adrenoceptors limits cardiovascular side effects, such as, for example, orthostatic hypotension. Accordingly, the present invention provides pharmaceutical compositions for treating a disease or disorder mediated through α₁ₐ and/or α_{1d} adrenoceptor subtypes. Compounds and pharmaceutical compositions described herein can be administered orally, parenterally, subcutaneously, transdermally or topically.

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, or —NR₁₄R₁₅, wherein R₁₄ and R₁₅ are selected from hydrogen, alkyl, alkenyl, cycloalkyl, cycloalkenyl, aryl, aralkyl, heterocyclyl, heteroaryl, heterocyclylalkyl, or heteroarylalkyl. Examples of alkyl include, but are not limited to, methyl, ethyl, propyl, isopropyl and butyl, and the like.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC(═O)R₁₄, —NR₁₄R₁₅, —C(═O)NR₁₄R₁₅, —NHC(═O)NR₁₄R₁₅, —O—C(═O)NR₁₄R₁₅ (wherein R₁₄ and R₁₅ are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or S(O)ₘRₕ (wherein m is an integer from 0-2 and Rₕ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkenyl substituents optionally may be substituted further by 1-3 substituents selected from alkyl, carboxy, hydroxy, alkoxy, halogen, —CF₃, cyano, —NR₁₄R₁₅, —C(═O)NR₁₄R₁₅, —O—C(═O)NR₁₄R₁₅ (wherein R₁₄ and R₁₅ are the same as defined earlier) and S(O)ₘRₕ (wherein m and Rₕ are the same as defined earlier).

The term “alkynyl,” unless otherwise specified, refers to a monoradical of an unsaturated hydrocarbon, having from 2 to 20 carbon atoms. In the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R₁₄, —NR₁₄R₁₅, —NHC(═O)NR₁₄R₁₅, —C(═O)NR₁₄R₁₅ (wherein R₁₄ and R₁₅ are the same as defined earlier), S(O)ₘRₕ (wherein m is an integer from 0-2 and Rₕ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR₁₄R₁₅, —C(═O)NR₁₄R₁₅, —NHC(═O)NR₁₄R₁₅ (wherein R₁₄ and R₁₅ are the same as defined earlier), cyano, or S(O)ₘRₕ (wherein m is an integer from 0-2 and Rₕ is same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR₁₄R₁₅, —NHC(═O)NR₁₄R₁₅, —NHC(═O)R₁₄, —C(═O)NR₁₄R₁₅, —O—C(═O)NR₁₄R₁₅ (wherein R₁₄ and R₁₅ are the same as defined earlier) nitro, heterocyclyl, heteroaryl, heterocyclylalkyl, heteroarylalkyl, or S(O)ₘRₕ (wherein m tis an integer from 0-2 and Rₕ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR₁₄R₁₅, —C(═O)NR₁₄R₁₅, —NHC(═O)NR₁₄R₁₅, —O—C(═O)NR₁₄R₁₅ (wherein R₁₄ and R₁₅ as defined earlier), cyano or S(O)ₘRₕ (wherein m is an integer from 0-2 and Rₕ is same as defined earlier).

The term “cycloalkenyl” refers to unsaturated carbocyclic ring having three to seven carbon atoms. Examples of cycloalkenyl include, but are not limited to, cyclopropenyl and cyclobutenyl, and the like. Cycloalkenyl groups may optionally be substituted with alkyl, halogen or hydroxy.

The term “halogen” refers to fluorine, chlorine, bromine or iodine.

The term “aryl,” unless otherwise specified, refers to carbocyclic aromatic groups, for example, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R₁₄, —NR₁₄R₁₅, —C(═O)NR₁₄R₁₅, —NHC(═O)NR₁₄R₁₅, —O—C(═O)NR₁₄R₁₅ (wherein R₁₄ and R₁₅ are the same as defined earlier), S(O)ₘRₕ (wherein m is an integer from 0-2 and R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “heterocycle” refers to non-aromatic or aromatic ring system having one or more heteroatom (s) wherein the said hetero atom (s) is/are selected from the group comprising of nitrogen, sulfur and oxygen and the ring system includes mono, bi or tricyclic. Examples of heterocycles include, but not limited to, azetidinyl, benzimidazolyl, 1,4-benzodioxanyl, 1,3-benzodioxolyl, benzoxazolyl, benzothiazolyl, benzothieenyl, dihydroimidazolyl, dihydropyranyl, dihydrofuranyl, dioxanyl, dioxolanyl, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, napthyridinyl, oxazolidinyl, oxazolyl, piperazinyl, piperidinyl, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, thiazolidinyl, thiazolyl, and thienyl, and the like.

Heterocycle groups may optionally be substituted with one or more substituent(s) independently selected from the group consisting of halogen, hydroxy, nitro, mercapto, cyano, alkyl, haloalkyl, alkoxy, haloalkoxy, thioalkyl, cycloalkoxy, —NR¹R², —CONR¹R², —COOR², —CONHR², —OCOR², —COR², —NHSO₂R² and —SO₂NHR² wherein R¹ and R² are independently selected from hydrogen or alkyl.

The term “alkoxy or cycloalkoxy” stands for a radical represented by Formula O-alkyl and O-cycloalkyl wherein alkyl and cycloalkyl are the same as defined above. Examples of alkoxy or cycloalkoxy include, but are not limited to, methoxy, ethoxy, propoxy, isopropoxy, cyclopentyloxy, and the like.

The term “thioalkyl” refers to S-alkyl wherein alkyl is the same as defined above.

The term “haloalkyl” stands for alkyl radical in which one or more hydrogen atom(s) is/are replaced by halogen atom(s). Examples of haloalkyl include, but are not limited to, trifluoromethyl, trifluoroethyl, tribromomethyl, chloro difluoro ethyl, and the like.

The term “haloalkoxy” refers to O-haloalkyl wherein haloalkyl is the same as defined above. Examples of haloalkoxy include, but are not limited to, trifluoromethoxy, trifluoroethoxy, chloro difluoro ethoxy, tetrafluoropropoxy and the like.

The present invention also encompasses prodrugs of the compounds disclosed herein. In general, such prodrugs will be functional derivatives of such compounds, which are readily convertible in vivo into the required compound. Conventional procedures for selecting and preparing suitable prodrug derivatives are described in, for example, “Design of Prodrugs”, ed. H. Bundgaard and, Elsevier, 1985.

The present invention also encompasses metabolites of the compounds disclosed herein, which become active upon introduction into a biological system.

Compounds disclosed herein possess two chiral centers and may therefore exist as enantiomers or diastereomers. It is to be understood that all such isomers or racemic mixtures therefore are encompassed within the scope of the present invention.

Crystalline or amorphous forms of compounds disclosed herein may exist as polymorphs and are encompassed in the present invention.

The compounds described herein may be prepared by techniques well known to one of ordinary skill in the art. In addition, the compounds described herein may be prepared by following the reaction sequences as shown in Schemes I, II, III, IV, V, VI, VII, VIII and IX below.

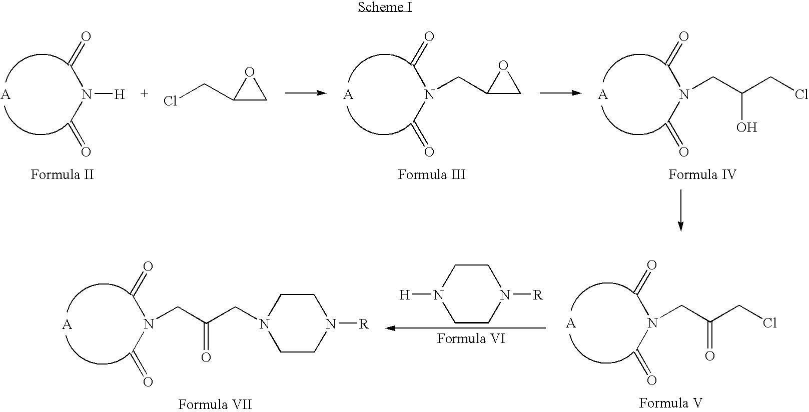

Compounds of Formula VII can be prepared according to Scheme I. Thus, compounds of Formula II can be reacted with 2-chloromethyl oxirane to form compounds of Formula III (wherein A is same as defined earlier). Compounds of Formula III can be reacted with hydrochloric acid to form compounds of Formula IV. Compounds of Formula IV can be oxidized to form compounds of Formula V, which on reaction with compounds of Formula VI form compounds of Formula VII (wherein R is same as defined earlier). Compounds of Formula VII can be further converted into their pharmaceutically acceptable salts using the methods well known to one of ordinary skill in art.

Compounds of Formula II can be reacted with 2-chloromethyl-oxirane in one or more solvents, for example, acetone, methyl ethyl ketone, diisopropyl ketone, tetrahydrofuran, dimethylformamide, dimethylsulfoxide or mixtures thereof. These reactions can also be carried out in the presence of one or more inorganic bases, for example, barium carbonate, cesium carbonate, calcium carbonate, sodium carbonate, potassium carbonate, sodium bicarbonate or a mixture thereof.

Compounds of Formula III can be reacted with hydrochloric acid in one or more solvents, for example, ethanol, methanol, isopropanol, ethyl acetate, tetrahydrofuran or mixtures thereof.

Compounds of Formula IV can be oxidized in one or more solvents, for example, chloroform, methanol, acetone, dichloromethane, acetonitrile, tetrahydrofuran or mixtures thereof. These reactions can also be carried out in the presence of one or more oxidizing agents, for example, pyridinium dichromate, pyridinium chlorochromate or mixtures thereof.

Compounds of Formula V can be reacted with compounds of Formula VI in one or more solvents, for example, acetonitrile, acetone, tetrahydrofuran, dimethylformamide, dimethylsulfoxide, toluene or mixtures thereof. These reactions can also be carried out in the presence of one or more inorganic bases, for example, barium carbonate, cesium carbonate, calcium carbonate, sodium carbonate, potassium carbonate, sodium bicarbonate or mixtures thereof.

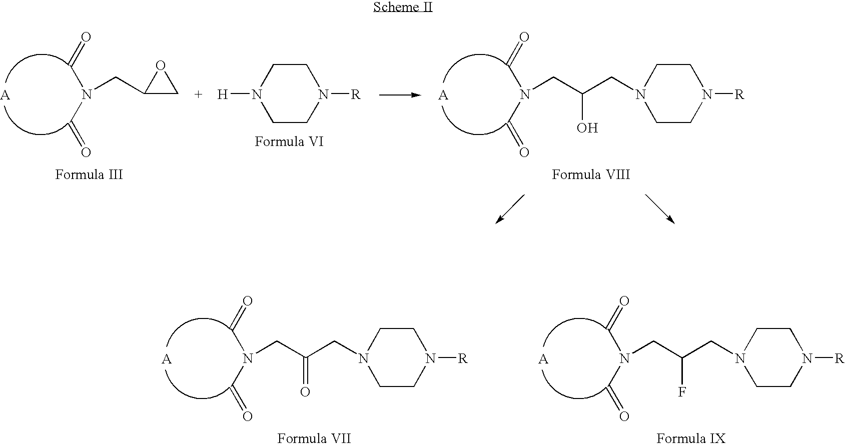

Compounds of Formula VII or IX can be prepared according to Scheme II. Thus, compounds of Formula III can be reacted with compounds of Formula VI to form compounds of Formula VIII (wherein A and R are the same as defined earlier). Compounds of Formula VIII can either be:
(a) oxidized to form compounds of Formula VII; or
(b) fluorinated to form compounds of Formula IX.

Compounds of Formulae VII or IX can be converted into their pharmaceutically acceptable salts using the methods known to one of ordinary skill in art.

Compounds of Formula III can be reacted with compounds of Formula VI in one or more solvents, for example, acetonitrile, acetone, ethanol, tetrahydrofuran, cyclohexane, dimethylformamide, dimethylsulfoxide, toluene, methylethylketone or mixtures thereof.

Compounds of Formula III can be reacted with compounds of Formula VI in the presence of one or more bases, for example, potassium carbonate, sodium carbonate, calcium carbonate, barium carbonate, sodium bicarbonate, triethyl amine, trimethyl amine, sodium hydride or mixtures thereof.

Compounds of Formula VIII can be fluorinated in the presence of one or more fluorinating agents, for example, diethylamino sulfur trifluoride, tris(dimethylamino)sulfur(trimethylsilyl)difluoride or mixtures thereof. These reactions can also be carried out in one or more solvents, for example, chloroform, dichloromethane, tetrahydrofuran, acetonitrile or mixtures thereof.

Compounds of Formula VIII can be oxidized in one or more solvents, for example, chloroform, methanol, acetone, dichloromethane, acetonitrile, tetrahydrofuran or mixtures thereof. These oxidation reactions can be carried out in the presence of one or more oxidizing agents, for example, pyridinium dichromate, pyridinium chlorochromate or mixtures thereof.

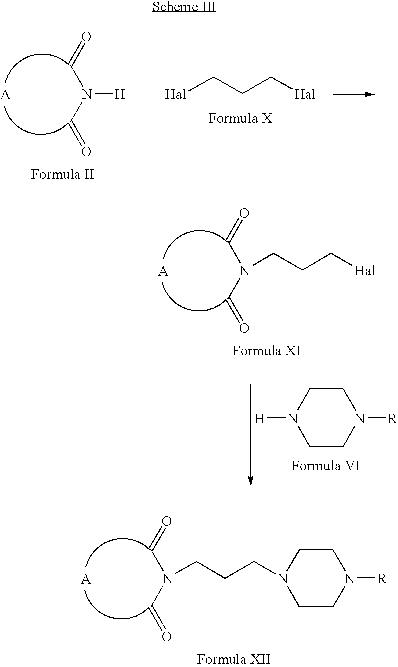

Compounds of Formula XII can be prepared according to Scheme III. Accordingly, Compounds of Formula II can be alkylated with compounds of Formula X to form compounds of Formula XI (wherein hal is a halogen and A is the same as defined earlier). Compounds of Formula XI can be reacted with compounds of Formula VI to form compounds of Formula XII (wherein R is the same as defined earlier). Compounds of Formula XII can be further converted into their pharmaceutically acceptable salts using the methods well known to one ordinary skilled in art.

Compounds of Formula II can be alkylated with compounds of Formula X in one or more solvents, for example, acetone, methyl ethylketone, diisopropyl ketone, tetrahydrofuran, dimethylformamide, dimethylsulfoxide or mixtures thereof. These alkylation reactions can also be carried out in the presence of one or more inorganic bases, for example, potassium carbonate, barium carbonate, cesium carbonate, calcium carbonate, sodium carbonate, sodium bicarbonate or mixtures thereof; and one or more organic or inorganic halides, for example, tetrabutyl ammonium chloride, tetrabutyl ammonium bromide, potassium iodide or mixtures thereof.

Compounds of Formula XI can be reacted with compounds of Formula VI in one or more solvents, for example, acetonitrile, ethanol, butanol, dichloromethane, dimethylformamide, dimethylsulfoxide or mixtures thereof. These reactions can also be carried out in the presence of one or more inorganic bases, for example, potassium carbonate, barium carbonate, cesium carbonate, calcium carbonate, sodium carbonate, sodium bicarbonate or mixtures thereof.

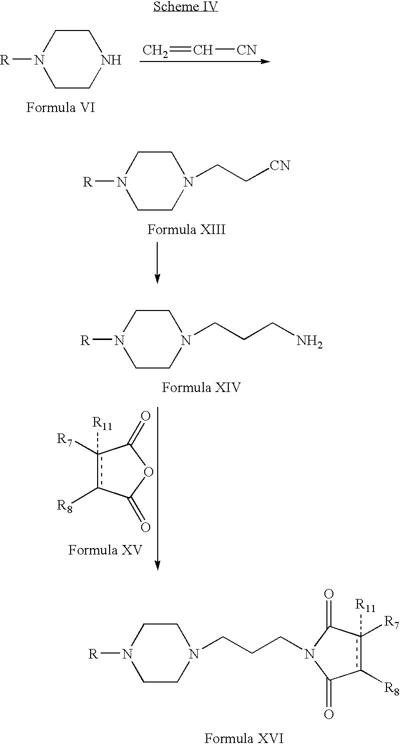

Compounds of Formula XVI can be prepared according to Scheme IV. Thus, reacting compounds of Formula VI with acrylonitrile form compounds of Formula XIII (wherein R is the same as defined earlier). Compounds of Formula XIII can be reduced to form compounds of Formula XIV. Compounds of Formula XIV can be reacted with compounds of Formula XV to form compounds of Formula XVI (wherein R₇, R₈ and R₁₁ are the same as defined earlier). Compounds of Formula XIV can be further converted into their pharmaceutically acceptable salts using the methods known to one of ordinary skill in art.

Compounds of Formula VI can be reacted with acrylonitrile in one or more alcoholic solvents, for example, methanol, ethanol, propanol, n-butanol or mixtures thereof.

Compounds of Formula XIII can be reduced in the presence of one or more reducing agents, for example, palladium on carbon and hydrogen; Raney nickel, hydrogen and ammonia in one or more alcoholic solvents, for example, methanol, ethanol, propanol, n-butanol or mixtures thereof; or mixtures thereof.

Compounds of Formula XIV can be reacted with compounds of Formula XV in one or more solvents, for example, toluene, tetrahydrofuran, acetonitrile, xylene or mixtures thereof.

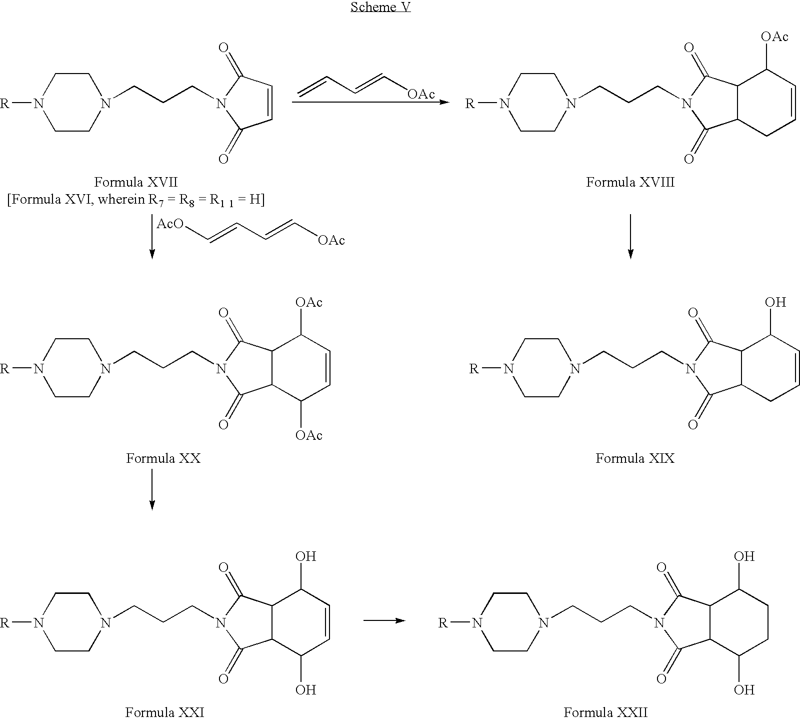

Compounds of Formula XIX or XXII can be prepared according to Scheme V. Thus, compounds of Formula XVII can be:

(a) reacted with 1-acetoxy-1,3-butadiene to form compounds of Formula XVIII, and such compounds of Formula XVIII can be hydrolyzed to form compounds of Formula XIX (wherein R is the same as defined earlier); or

(b) reacted with l,4-diacetoxy-1,3-butadiene to form compounds of Formula XX; such compounds of Formula XX can be hydrolyzed to form compounds of Formula XXI; and such compounds of Formula XXI can be reduced to form compounds of Formula XXII (wherein R is the same as defined earlier).

Compounds of Formula XIX or XXII can be further converted into their pharmaceutically acceptable salts using methods known to one of ordinary skill in art.

Compounds of Formula XVII can be reacted with l-acetoxy-1,3-butadiene or 1,4-diacetoxy-1,3-butadiene in one or more solvents, for example, toluene, benzene, xylene or mixtures thereof.

Compounds of Formula XVIII or Formula XX can be hydrolyzed in the presence of hydrochloric acid in one or more alcoholic solvents, for example, methanol, ethanol, propanol, n-butanol or mixtures thereof.

Compounds of Formula XXI can be reduced in the presence of one or more reducing agents, for example, palladium on carbon and hydrogen; Raney nickel, hydrogen and ammonia in one or more alcoholic solvents, for example, methanol, ethanol, propanol or n-butanol; or mixtures thereof.

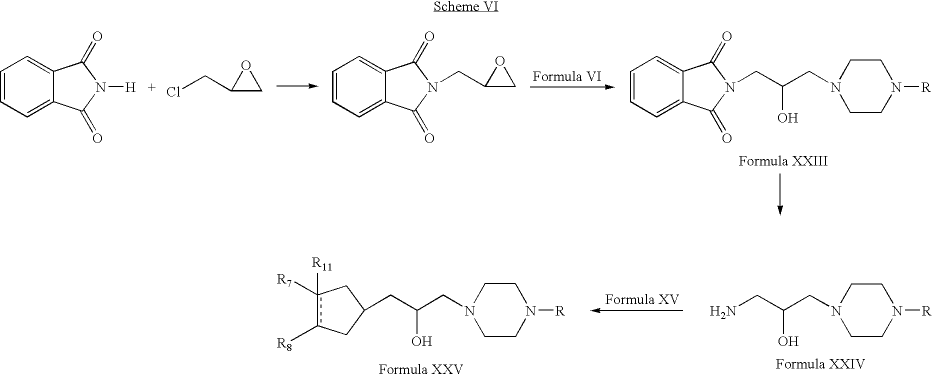

Compounds of Formula XXV can be prepared according to Scheme VI. Thus, isoindole-1,3-dione can be reacted with 2-chloromethyl oxirane to form 2-oxiranylmethyl-isoindole-1,3-dione. 2-oxiranylmethyl-isoindole-1,3-dione can be reacted with compounds of Formula VI to form compounds of Formula XXIII (wherein R is the same as defined earlier). Compounds of Formula XXIII with hydrazine hydrate to form compounds of Formula XXIV. Compounds of Formula XXIV can be reacted with compounds of Formula XV to form compounds of Formula XXV (wherein R₇, R₈ and R₁₁ are the same as defined earlier). Compounds of Formula XXV can be further converted into their pharmaceutically acceptable salts using the methods well known to one of ordinary skill in the art.

Isoindole-1,3-dione can be reacted with 2-chloromethyl-oxirane in one or more solvents, for example, acetone, methyl ethyl ketone, diisopropyl ketone, tetrahydrofuran, dimethylformamide, dimethylsulfoxide or mixtures thereof. The reaction can also be carried out in the presence of one or more inorganic bases, for example, barium carbonate, cesium carbonate, calcium carbonate, sodium carbonate, potassium carbonate, sodium bicarbonate or mixtures thereof.

2-oxiranylmethyl-isoindole-1,3-dione can be reacted with compounds of Formula VI in one or more organic solvents, for example, acetonitrile, ethanol, butanol, tetrahydrofuran, dimethylsulphoxide, dimethylformamide, dichloromethane or mixtures thereof.

Compounds of Formula XXIII can be reacted with hydrazine hydrate in one or more solvents, for example, acetonitrile, ethanol, butanol, tetrahydrofuran, dimethylsulphoxide, dimethylformamide, dichloromethane or mixtures thereof.

Compounds of Formula XXIV can be reacted with compounds of Formula XV in one or more solvents, for example, acetonitrile, acetone, tetrahydrofuran, dimethylformamide, dimethylsulfoxide, toluene or mixtures thereof.

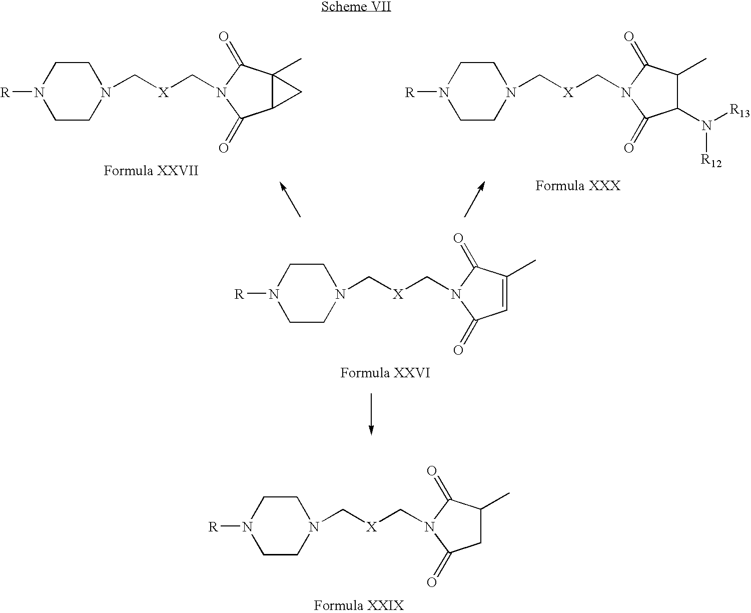

Compounds of Formula XXVII, XXIX or XXX can be prepared according to Scheme VII. Thus,

(a) compounds of Formula XXVI can be reacted with one or more methylating agents, for example, trimethyl sulphoxinium iodide, to form compounds of Formula XXVII (wherein X is the same as defined earlier);

(b) compounds of Formula XXVI can be reduced to form compounds of Formula XXIX (wherein X is the same as defined earlier); or

(c) compounds of Formula XXVI can be reacted with compounds of Formula XXVIII (wherein X, R₁₂ and R₁₃ are the same as defined earlier) to form compounds of Formula XXX.

Compounds of Formula XXVII, XXIX or XXX can be further converted into their pharmaceutically acceptable salts using methods known to one of ordinary skill in the art.

Compounds of Formula XXVI can be reacted with a methylating agent in one or more solvents, for example, acetonitrile, acetone, tetrahydrofuran, dimethylformamide, dimethylsulfoxide, toluene or mixtures thereof.

Compounds of Formula XXVI can be reduced in the presence of one or more reducing agents, for example, palladium on carbon and hydrogen; Raney nickel, hydrogen and ammonia in one or more alcoholic solvents, for example, methanol, ethanol, propanol, n-butanol or mixtures thereof; or mixtures thereof.

Compounds of Formula XXVI can be reacted with compounds of Formula XXVIII in one or more solvents, for example, chloroform, methanol, acetone, dichloromethane, acetonitrile, tetrahydrofuran or mixtures thereof.

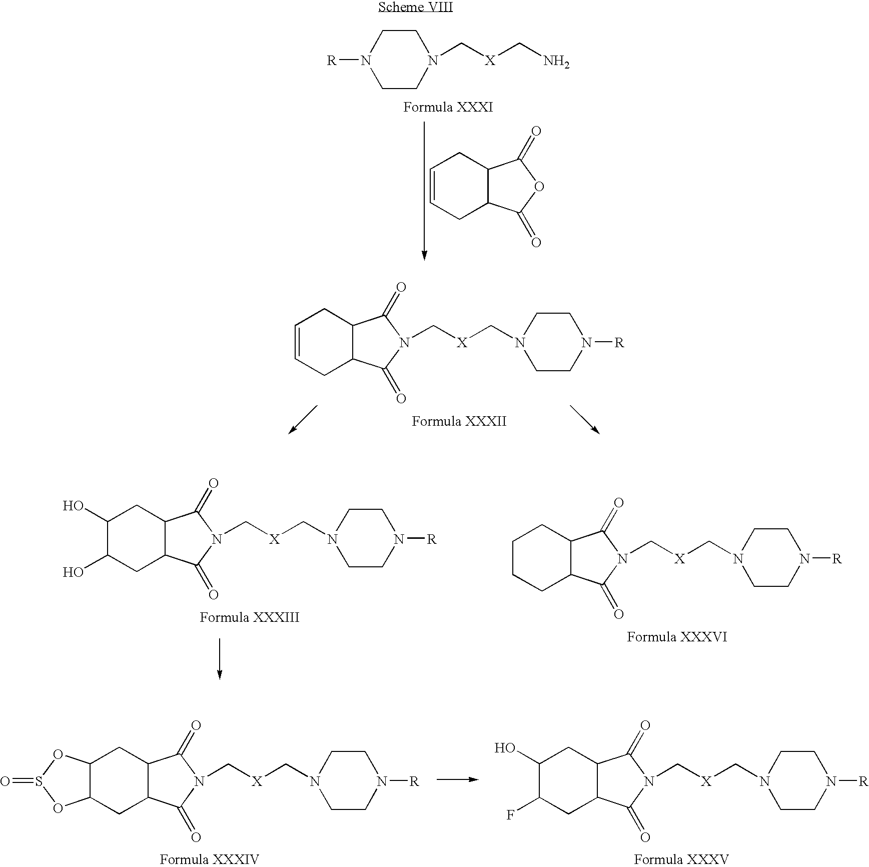

Compounds of Formula XXXV OR XXXVI can be prepared according to Scheme VIII. Thus, compounds of Formula XXXI can be reacted with tetrahydrophthalimide to form compounds of Formula XXXII (wherein X and R are the same as defined earlier). Compounds of Formula XXXII can be:

(a) oxidized to form compounds of Formula XXXIII; compounds of Formula XXXIII can be reacted with diethylaminosulfur trifluoride to form compounds of Formula XXXIV; compounds of Formula XXXIV can be reacted with diethylaminosulfur trifluoride to form compounds of Formula XXXV; or

(b) reduced to form compounds of Formula XXXVI.

Compounds of Formula XXXV or XXXVI can be further converted into their pharmaceutically acceptable salts using methods known to one of ordinary skill in the art.

Compounds of Formula XXXI can be reacted with tetrahydrophthalimide in one or more solvents, for example, acetonitrile, acetone, tetrahydrofuran, dimethylformamide, dimethylsulfoxide, toluene or mixtures thereof.

Compounds of Formula XXXII can be oxidized in one or more alcoholic solvents, for example, methanol, ethanol, propanol, n-butanol or mixtures thereof, in the presence of one or more oxidizing agents, for example, potassium permanganate.

Compounds of Formula XXXII can be reacted with diethylaminosulfur trifluoride in more than one solvents, for example, chloroform, dichloromethane, tetrahydrofuran, acetonitrile or mixtures thereof.

Compounds of Formula XXXIV can be reacted with diethylaminosulfur trifluoride in one or more solvents, for example, chloroform, dichloromethane, tetrahydrofuran, acetonitrile or mixtures thereof.

Compounds of Formula XXXII can be reduced in the presence of one or more reducing agents, for example, palladium on carbon and hydrogen; Raney nickel, hydrogen in one or more alcoholic solvents, for example, methanol, ethanol, propanol, n-butanol or mixtures thereof; or mixtures thereof.

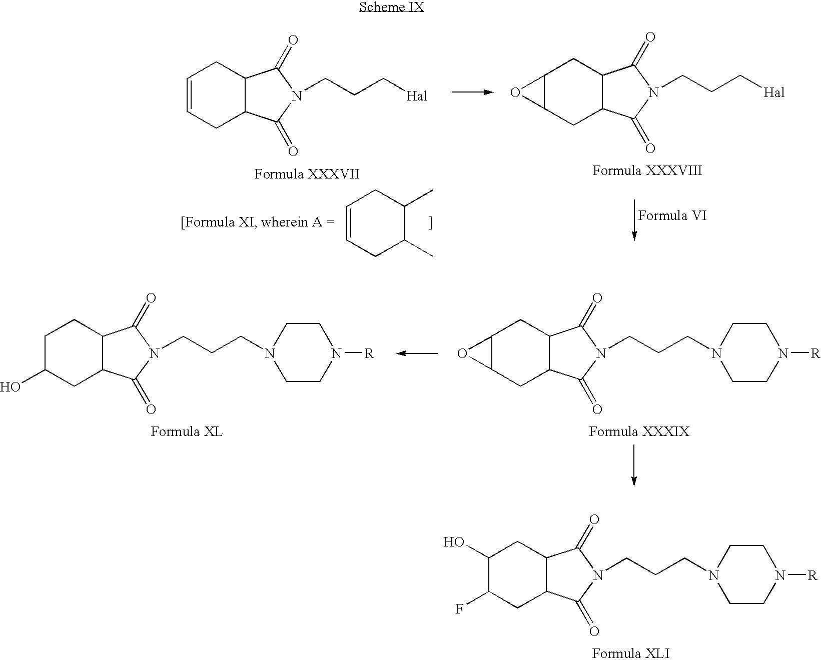

Compounds of Formula XL or XLI can be prepared according to Scheme IX. Thus, compounds of Formula XXXVII (wherein hal is a halogen) can be reacted with one or more peroxyacids, for example, m-chloroperbenzoic acid, to form compounds of Formula XXXVIII. Compounds of Formula XXXVIII can be reacted with compounds of Formula VI to form compounds of Formula XXXIX (wherein R is the same as defined earlier). Compounds of Formula XXXIX can be:

(a) reduced to form compounds of Formula XL; or

(b) fluorinated to form compounds of Formula XLI.

Compounds of Formula XL or XLI can be converted into their pharmaceutically acceptable salts using methods known to one of ordinary skill in the art.

Compounds of Formula XXXVII can be reacted with one or more peroxyacids in one or more solvents, for example, chloroform, methanol, acetone, dichloromethane, acetonitrile, tetrahydrofuran or mixtures thereof.

Compounds of Formula XXXVIII can be reacted with compounds of Formula VI in one or more solvents, for example, acetonitrile, ethanol, butanol, halogenated solvents, tetrahydrofuran, dimethylformamide, dimethylsulfoxide or mixtures thereof. These reactions can also be carried out in the presence of one or more inorganic bases, for example, potassium carbonate, barium carbonate, cesium carbonate, calcium carbonate,

sodium carbonate, sodium bicarbonate or mixtures thereof.

Compounds of Formula XXXIX can be reduced in the presence of one or more reducing agents, for example, palladium on carbon and hydrogen; Raney nickel or hydrogen in one or more solvents, for example, chloroform, methanol, acetone, dichloromethane, acetonitrile, tetrahydrofuran or mixtures thereof; or mixtures thereof.

Compounds of Formula XXXIX can be fluorinated in the presence of one or more fluorinating agents, for example, diethylamino sulfur trifluoride, tris(dimethylamino)sulfur(trimethyl silyl) di fluoride or mixtures thereof, in one or more solvents, for example, chloroform, dichloromethane, tetrahydrofuran, acetonitrile or mixtures thereof.

The compounds described herein are basic and can form organic or inorganic acid addition salts, which can be suitably administerable in humans and other animals without undue toxicity, irritation, allergic response, and the like. The resulting addition salts are useful alone or in pharmaceutical compositions. These salts may be prepared by methods known to one of ordinary skill in the art, for example, suspending the compound in water and then adding one equivalent of one or more organic acids, e.g., acetic acid, oxalic acid, maleic acid, tartaric acid, citric acid, succinic acid, malonic acid, adipic acid, ascorbic acid, camphoenic acid, nicotinic acid, butyric acid, lactic acid, glucuronic acid or mixtures thereof, and/or one or more inorganic acids, e.g., hydrochloric acid, hydrobromic acid, phosphoric acid, sulfuric acid, nitric acid, boric acid, perchloric acid or mixtures thereof.

Neutral solutions of addition salts can be subjected to rotary evaporation under reduced pressure to volumes sufficient to facilitate precipitation of the salt upon cooling, which is then filtered and dried. The salts of the present invention may also be prepared under strictly non-aqueous conditions. For example, free base can be dissolved in one or more suitable organic solvents, for example, ethanol, methanol, isopropanol, dichloromethane, diethyl ether or mixtures thereof, to form a solution; one equivalent of a suitable acid can be added to the solution; and the solution can be stirred at temperatures of between about 0° C. to 5° C., precipitating corresponding acid addition salts, which can then be filtered, washed with one or more solvents and dried. In another example, solvent can be completely removed by reduced pressure to obtain addition salts. Such salts are typically preferable for use in formulating pharmaceutical compositions of the invention because they are crystalline, relatively more stable and water-soluble.

Compounds described herein can be administered to a patient (e.g., human or animal) orally, parenterally, topically, rectally, internasally, subcutaneously or transdermally. Pharmaceutical compositions of the present invention can comprise pharmaceutically effective amounts of one or more compounds of the present invention formulated together with one or more pharmaceutically acceptable carriers.

The term “pharmaceutically acceptable carriers” is intended to include non-toxic, inert solid, semi-solid or liquid filter, diluent, encapsulating material or formulation auxiliary of any type.

Solid form preparations for oral administration, include capsules, tablets, pills, powder, granules, cachets or suppositories. For solid form preparations, one or more active compounds can be mixed with one or more inert, pharmaceutically acceptable excipients or carriers, for example, sodium citrate, dicalcium phosphate and/or one or more fillers or extenders, for example, starch, lactose, sucrose, glucose, mannitol, silicic acid or mixtures thereof; one or more binders, for example, carboxymethylcellulose, alginates, gelatins, polyvinylpyrolidinone, sucrose, acacia or mixtures thereof; disintegrating agents, for example, agar-agar, calcium carbonate, potato starch, alginic acid, certain silicates, sodium carbonate or mixtures thereof; absorption accelators, for example, quaternary ammonium compounds; wetting agents, for example, cetyl alcohol, glycerol, monostearate or mixtures thereof; adsorbents, for example, kaolin; lubricants, for example, talc, calcium stearate, magnesium stearate, solid polyethyleneglycol, sodium lauryl sulfate or mixtures thereof.

For capsules, tablets or pills, dosage forms can also comprise one or more buffering agents.

Solid preparations of tablets, capsules, pills or granules can also be prepared with one or more coatings and/or shells, for example, enteric coating and other coatings well known in the pharmaceutical formulating art.

Liquid form preparations for oral administration include pharmaceutically acceptable emulsions, solutions, suspensions, syrups or elixirs. For liquid form preparations, one or more active compounds can be mixed with water and/or other solvent(s), one or more solubilizing agents or emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor or sesame oil), glycerol, fatty acid esters of sorbitan or mixtures thereof. In addition to inert diluents, oral compositions can also include one or more adjuvants, for example, wetting agents, emulsifying agents, suspending agents, sweetening agents, flavoring agents, perfuming agents or mixtures thereof.

Injectable preparations (e.g., sterile injections, aqueous or oleaginous suspensions) may be formulated according to methods known to one of ordinary skill in the art, for example, using one or more suitable dispersing agents, wetting agents, suspending agents or mixtures thereof. Acceptable carriers or solvents that may be employed include, for example, water, Ringer's solution, U.S.P., isotonic sodium chloride or mixtures thereof.

Dosage forms for topical or transdermal administration include ointments, pastes, creams, lotions, gel, powders, solutions, spray, inhalants or patches. Active compound can be admixed under sterile conditions with one or more pharmaceutically acceptable carriers, as well as any preservatives or buffers as may be required. Ophthalmic formulations, eardrops, eye ointments, powders and solutions are also encompassed within the scope of this invention.

Pharmaceutical preparations may be in unit dosage form. In particular, preparations may be subdivided into unit dosage forms containing appropriate and therapeutically effective quantities of one or more active ingredients. Unit dosage forms can be packaged preparations containing discrete capsules, powders, in vials or ampoules, ointments, capsules, cachets, tablets, gels, creams, or any combination thereof and in appropriate numbers of unit dosages.

Formulations of the present invention may be formulated by methods known to one of ordinary skill in the art to provide immediate release, as well as sustained- or delayed-release of active ingredients after administration to a patient.

Compounds described herein, bladder selective muscarinic receptor antagonists and/or 5α reductase inhibitors can be formulated in combination to achieve desired therapeutic effects, i.e., combination therapies. As such, the dosage amounts of such active ingredients can be adjusted accordingly, without undue experimentation and well within the abilities of one of ordinary skill in the art. As one of ordinary skill in the art can appreciate, dosage amounts of compounds described herein, bladder selective muscarinic receptor antagonists and/or 5a reductase inhibitors may be independently optimized and combined to achieve a synergistic therapeutic result. In accordance with methods encompassed herein, individual components of any combination can be administered separately in any sequence at the same or different times during the course of therapy, or concurrently in divided or single combination forms.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### Examples

### Example 1

### Preparation of 2-{3-[4-(2-Isopropoxy-phenyl)-piperazin-1-yl]-2-oxo-propyl}-3a,4,7,7a-tetrahydro-isoindole-1,3-dione hydrochloride salt (Compound No. 6)

### Step 1: Preparation of 2-oxiranylmethyl-3a,4,7,7a-tetrahydro-isoindole-1,3-dione

A solution of cis-1,2,3,6-tetrahydrophthalimide (5 gm, 32.89 mmol), epichlorohydrin (6.0 gm, 65.7 mmol), and potassium carbonate (9.0 gm, 65.7 mmol) in methyl ethyl ketone (30 mL) was refluxed. After completion of the reaction, the reaction mixture was filtered through G-4 and washed with methyl ethyl ketone. The filtrate was concentrated to yield a thick residue. Water was added to the residue, extracted with ethyl acetate, dried over anhydrous sodium sulfate and concentrated to yield the crude product. The crude product was purified on silica gel column using dichloromethane as eluent to yield 2-oxiranylmethyl-3a,4,7,7a-tetrahydro-isoindole-1,3-dione. Yield: 5.0 g (74%)

### Step 2: Preparation of 2-(3-chloro-2-hydroxy-propyl)-3a,4,7,7a-tetrahydro-isoindole-1,3-dione

To a solution of 2-oxiranylmethyl-3a,4,7,7a-tetrahydro-isoindole-l, 3-dione (4.0 gm, 19.23 mmol) in ethanol was added ethanolic hydrochloride and reaction mixture stirred. The reaction mixture was then neutralized with sodium bicarbonate. Inorganics were then filtered and washed with ethanol. The filtrate was concentrated to yield 2-(3-chloro-2-hydroxy-propyl)-3a,4,7,7a-tetrahydro-isoindole-1,3-dione . Yield: 4.2 g (89.36%)

### Step 3: Preparation of 2-(3-chloro-2-oxo-propyl)-3a,4,7,7a-tetrahydro-isoindole-1,3-dione

To a solution of 2-(3-chloro-2-hydroxy-propyl)-3a,4,7,7a-tetrahydro-isoindole-1,3-dione (2.0 gm, 8.17 mmol) in dichloromethane was added pyridinium chlorochromate (3.5 g, 16.35 mmol) and the reaction mixture was refluxed. After completion of the reaction, the reaction mixture was filtered through a celite pad and washed with dichloromethane. The filtrate was concentrated to yield the crude product, which was then purified on a silica gel column using dichloromethane:methanol as eluent to yield 2-(3-chloro-2-oxo-propyl)-3a,4,7,7a-tetrahydro-isoindole-1,3-dione. Yield: 1.5 g (75%)

### Step 4: Preparation of 2-{3-[4-(2-Isopropoxy-phenyl)-piperazin-1-yl]-2-oxo-propyl}-3a,4,7,7a-tetrahydro-isoindole-1,3-dione

A solution of 2-(3-chloro-2-oxo-propyl)-3a,4,7,7a-tetrahydro-isoindole-1,3-dione (1.0 gm, 4 mmol), 2-isopropoxyphenyl piperazine (0.91 gm, 4 mmol), potassium carbonate (0.57 gm, 4 mmol) in dimethylformamide was heated. The reaction was quenched by adding water and extracted with ethyl acetate. The organic layer was dried over anhydrous sodium sulfate and concentrated to yield the crude product, which was then purified on silica gel column using dichloromethane:methanol as eluent to yield 2-(3-[4-(2-Isopropoxy-phenyl)-piperazin-1-yl]-2-oxo-propyl}-3a,4,7,7a-tetrahydro-isoindole-1,3-dione. Yield: 1.2 gm (69%)

The following compound was also prepared following the above procedure:
Compound No. 21: 2-{3-[4-(2-Cyclopentyloxy-phenyl)-piperazin-1-yl]-2-oxo-propyl}-3a,4,7,7a-tetrahydro-isoindole-1,3-dione
IR (KBr): 1703.9 cm⁻¹; ¹H NMR (300 MHz, DMSO-d₆): δ 1.61-1.74 (m, 8H), 2.23-2.27 (m, 2H), 2.37-2.42 (m, 2H), 3.04 (brs, 2H), 3.18-3.47 (m, 8H), 4.50 (s, 2H), 4.62-4.65 (brs, 2H), 4.83 (brs, 1H), 5.88 (brs, 2H), 6.87-7.00 (m, 4H), 10.5 (brs, 1H); Mass (m/z): 452.3 (M⁺1)

The following compounds were similarly prepared
Compound No. 32: 1-[2-Oxo-3-(4-phenyl-piperazin-1-yl)-propyl]-pyrrolidine-2,5-dione
Compound No. 33: 1-{3-[4-{4-Fluoro-phenyl}-piperazin-1-yl]-2-oxo-propyl}-3-phenyl-piperidine-2,6-dione
Compound No. 34: 3,4-Dimethyl-1-{2-oxo-3-[4-(2-trifluoromethyl-phenyl)-piperazin-1-yl]-propyl}-pyrrole-2,5-dione

### Step 5: Preparation of 2-{3-[4-(2-Isopropoxy-phenyl)-piperazin-1-yl]-2-oxo-propyl}-3a,4,7,7a-tetrahydro-isoindole-1,3-dione hydrochloride salt

To a solution of 2-{3-[4-(2-Isopropoxy-phenyl)-piperazin-1-yl]-2-oxo-propyl}-3a,4,7,7a-tetrahydro-isoindole-1,3-dione (0.5 gm, 1 mmol) in isopropyl alcohol was added isopropyl alcohol/hydrochloric acid at 10-15° C. and the reaction mixture was stirred for about 1 hr. A solid precipitate was filtered, dried and weighed to yield 2-{3-[4-(2-Isopropoxy-phenyl)-piperazin-1-yl]-2-oxo-propyl}-3a,4,7,7a-tetrahydro-isoindole-1,3-dione hydrochloride salt. Yield: 0.45 gm (83%)
IR (KBr): 1746.7, 1705.0 cm⁻¹; ¹H NMR (300 MHz, DMSO-d₆): δ 1.26-1.28 (d, 6H), 2.22-2.26 (d, 2H), 2.36-2.41 (d, 2H), 3.05-3.47 (m, 10H), 4.49-4.64 (m, 5H), 5.89 (brs, 2H), 6.90-6.95 (m, 4H), 10.40 (brs, 1H); Mass (m/z): 426 (M⁺+1)

The following compounds were prepared following the above procedure
Compound No. 20: 2-{3-[4-(2-Methoxy-phenyl)-piperazin-1-yl}-2-oxo-propyl}-3a,4,7,7a-tetrahydro-isoindole-1,3-dione hydrochloride salt
IR (KBr): 1707.4 cm⁻¹; ¹H NMR (300 MHz, DMSO-d₆): δ 2.22-2.27 (m, 2H), 2.36-2.42 (m, 2H), 3.05-3.17 (m, 2H), 3.25-3.36 (m, 8H), 3.78 (s, 3H), 4.49-4.59 (m, 4H), 5.89 (brs, 2H), 6.90-7.02 (m, 4H), 10.60 (brs, 1H); Mass (m/z): 398.3 (M⁺+1)
Compound No. 23: 2-{2-Oxo-3-[4-(2-propoxy-phenyl)-piperazin-1-yl]-propyl}-3a,4,7,7a-tetrahydro-isoindole-1,3-dione hydrochloride salt
IR (KBr): 1704.7 cm¹; ¹H NMR (300 MHz, DMSO-d₆): δ 0.99-1.04 (t, 3H), 1.71-1.80 (m, 2H), 2.23-2.27 (m, 2H), 2.37-2.42 (m, 2H), 3.08 (m, 2H), 3.17-3.48 (m, 8H), 3.91-3.95 (m, 2H), 4.50 (s, 2H), 4.63 (s, 2H), 5.89 (brs, 2H), 6.86-7.02 (m, 4H), 10.65 (brs, 1H); Mass (m/z): 426.5 (M⁺+1)
Compound No. 25: 2-{3-[4-(4-Fluoro-2-isopropoxy-phenyl)-piperazin-1-yl]-2-oxo-propyl}-3a,4,7,7a-tetrahydro-isoindole-1,3-dione hydrochloride salt
IR (KBr): 1707.5 cm⁻¹; ¹H NMR (300 MHz, DMSO-d₆): δ 1.27-1.29 (d, 6H), 2.22-2.27 (m, 2H), 2.36-2.41 (m, 2H), 3.06 (brs, 2H), 3.25-3.38 (m, 8H), 4.49-4.66 (m, 5H), 5.88-5.89 (d, 2H), 6.67-6.68 (m, 1H), 6.85-6.89 (m, 3H), 10.80 (brs, 1H); Mass (m/z): 444.3(M⁺1)
Compound No. 27: 2-{3-[4-(2-Isopropoxy-phenyl)-piperazin-1-yl]-2-oxo-propyl}-isoindole-1,3-dione hydrochloride salt
IR (KBr): 1727.1 cm⁻¹; ¹H NMR (300 MHz, DMSO-d₆): δ 1.27-1.29 (d, 6H), 3.10-3.49 (m, 8H), 4.59-4.65 (m, 1H), 4.74 (s, 4H), 6.88-6.98 (m, 4H), 7.89-7.97 (m, 4H), 10.87 (brs, 1H); Mass (m/z): 422.5 (M⁺+1)
Compound No. 29: 2-(2-Oxo-3-{4-[2-(2,2,2-trifluoro-ethoxy)-phenyl]-piperazin-1-yl}-propyl)-3a,4,7,7a-tetrahydro-isoindole-1,3-dione hydrochloride salt
IR (KBr): 1704.2 cm⁻¹; ¹H NMR (300 MHz, DMSO-d₆): δ 2.22-2.41 (m, 4H), 3.10-3.31 (m, 10H), 4.48 (brs, 2H), 4.61 (brs, 2H), 4.68-4.77 (m, 2H), 5.88 (s, 2H), 7.01-7.04 (m, 2H), 10.50 (brs, 1H); Mass (m/z): 466.5 (M⁺+1)

### Example 2

### Preparation of 2-(2-Hydroxy-3-[4-(2-isopropoxy-phenyl)-piperazin-1-yl]-propyl}-hexahydro-isoindole-1,3-dione hydrochloride salt (Compound No. 2)

### Step 1: Preparation of 2-{2-Hydroxy-3-[4-(2-isopropoxy-phenyl)-piperazin-1-yl]-propyl}-3a,4,7,7a-tetrahydro-isoindole-1,3-dione hydrochloride salt

A solution of 2-oxiramylmethyl-3a,4,7,7a-tetrahydroisoindole-1,3-dione (4.0 gm, 19.2 mmol), 2-isopropoxyphenyl piperazine monohydrochloride (4.9 g, 19.2 mmol), potassium carbonate (5.3 gm, 38.4 mmol) in dimethylformamide was heated. The reaction was quenched by adding water, extracted with ethyl acetate, dried over anhydrous sodium sulfate and concentrated to yield the crude product. The crude product was purified on silica gel column using dichloromethane-methanol as eluent to yield 2-{2-Hydroxy-3-[4-(2-isopropoxy-phenyl)-piperazin-1-yl]-propyl}-3a,4,7,7a-tetrahydro-isoindole-1,3-dione hydrochloride salt. Yield: 7.0 gm (85%)

### Step 2: Preparation of 2-{2-Hydroxy-3-[4-(2-isopropoxy-phenyl)-piperazin-1-yl]-propyl}-hexahydro-isoindole-1,3-dione hydrochloride salt

A solution of 2-{2-Hydroxy-3-[4-(2-isopropoxy-phenyl)-piperazin-1-yl]-propyl}-3a,4,7,7a-tetrahydro-isoindole-1,3-dione (2.0 g, 4.6 mmol) in methanol was hydrogenated with palladium/carbon on hydrogen. After completion of the reaction, the reaction mixture was filtered through a celite pad, washed with methanol, and the filtrate was concentrated to yield 2-{2-Hydroxy-3-[4-(2-isopropoxy-phenyl)-piperazin-1-yl]-propyl}-hexahydro-isoindole-1,3-dione hydrochloride salt. Yield: 1.9 gm (95%)
IR (KBr): 1699.0, 1671.2 cm⁻¹; ¹H NMR (300 MHz, CDCl₃): δ 1.33-1.35 (d, 6H), 1.46 (brs, 4H), 1.84-1.85 (m, 4H), 2.43-2.46 (m, 2H), 2.60-2.63 (m, 2H), 2.80-2.85 (m, 4H), 3.10 (brs, 4H), 3.53-3.68 (m, 2H), 4.01-4.04 (m, 1H), 4.56-4.60 (m, 1H), 6.84-6.92 (m, 4H), 9.96 (brs, 1H); Mass (m/z): 430.1 (M⁺+1)

The following compounds were prepared following the above procedure
Compound No. 8: 2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione hydrochloride salt
IR (KBr): 1697.4 cm⁻¹; ¹H NMR (300 MHz, DMSO-d₆): δ 2.21-2.25 (d, 2H), 2.44-2.56 (d, 2H), 3.02-3.20 (m, 8H), 3.37-3.63 (m, 6H), 4.16 (m, 1H), 4.63-4.72 (m, 2H), 5.89 (brs, 2H), 7.01-7.05 (m, 4H), 10.42 (brs, 1H); Mass (m/z): 468.2 (M⁺+1)
Compound No. 10: 2-(2-Hydroxy-3-{4-[2-(2,2,3,3-tetrafluoro-propoxy)-phenyl]-piperazin-1-yl}-propyl)-3a,4,7,7a-tetrahydro-isoindole-1,3-dione hydrochloride salt
IR (KBr): 1695.3 cm⁻¹; ¹H NMR (300 MHz, DMSO-d₆): δ 2.19-2.24 (m, 2H), 2.36-2.41 (m, 2H), 3.01-3.16 (m, 8H), 3.41-3.50 (m, 6H), 3.62-3.65 (m, 1H), 4.20 (m, 1H), 4.54-4.63 (m, 2H), 5.88 (brs, 2H), 6.49-6.68 (m, 1H), 7.01-7.08 (m, 4H), 10.14 (brs, 1H); Mass (m/z): 500.3. (M⁺+1)
Compound No. 12: 2-{2-Hydroxy-3-[4-(2-isopropoxy-4-nitro-phenyl)-piperazin-1-yl]-propyl}-3a,4,7,7a-tetrahydro-isoindole-1,3-dione hydrochloride salt
IR (KBr): 1697.4 cm⁻¹; ¹H NMR (300 MHz, CDCl₃): δ 1.41-1.43 (d, 6H), 2.01 (m, 2H), 2.20-2.25 (m, 2H), 3.16 (m, 6H), 3.46-3.49 (m, 6H), 4.01 (m, 2H), 4.71 (m, 2H), 5.89 (brs, 2H), 6.97 (d, 1H), 7.71 (s, 1H), 7.83 (brs, 1H), 11.40 (brs, 1H); Mass (m/z): 472.7 (M⁺+1)
Compound No. 68: 2-{3-[4-(2-Cyclopentyloxy-phenyl)-piperazin-1-yl]-2-hydroxy-propyl}-hexahydro-isoindole-1,3-dione hydrochloride salt
IR (KBr): 1701.9 cm⁻¹; ¹H NMR (300 MHz, CDCl₃): δ 1.446 (s, 4H), 1.668-2.008 (m, 12H), 2.981-3.153 (m, 4H), 3.447 (s, 6H), 3.524-3.586 (m, 2H), 3.677-3.722 (q, 2H), 4.541 (s, 1H), 4.713-4.806 (d, 2H), 6.842-7.004 (m, 4H); Mass (m/z): 456 (M⁺+1)
Compound No. 80: 2-{3-[4-(2-Cyclopentyloxy-5-fluoro-phenyl)-piperazin-1-yl]-2-hydroxy-propyl}-hexahydro-isoindole-1,3-dione hydrochloride salt
IR (KBr): 1701cm⁻¹; ¹H NMR (300 MHz, CDCl₃): δ 1.456-1.860(m, 16H), 2.922-2.969(d, 4H), 3.223-3.356(d, 8H), 3.532-3.671(m, 2H), 4.415(s, 1H), 4.738(s, 2H), 6.623-6.762 (m, 3H); Mass (m/z): 474 (M⁺+1)
Compound No. 88: 1-{3-[4-(5-Fluoro-2-isopropoxy-phenyl)-piperazin-1-yl]-2-hydroxy-propyl}-piperidine-2,6-dione hydrochloride salt
IR (KBr): 1653.3 cm⁻¹; ¹H NMR (300 MHz, CDCl₃): δ 1.31-1.33 (d, 6H), 1.96-2.00 (t, 2H), 2.66-2.71(t, 4H), 3.26 (s, 3H), 3.48-3.66 (t, 9H), 4.04-4.06 (d, 2H), 4.44-4.50 (m, 1H), 6.63-6.69 (m,3H); Mass (m/z): 408 (M+1)
Compound No. 90: 1-{3-[4-(2-Cyclopentyloxy-5-fluoro-phenyl)-piperazin-1-yl]-2-hydroxy-propyl}-piperidine-2,6-dione hydrochloride salt
IR (KBr): 1668.2 cm⁻¹; ¹H NMR (300 MHz, CDCl₃): δ 1.65-1.98 (m, 11H), 2.72 (s, 3H), 2.81(s, 1H), 3.51-3.94(m, 12H), 4.03-4.09(m, 1H), 4.73(s, 1H), 6.63-6.77(m, 3H); Mass (m/z): 767 (M⁺+1)
Compound No. 100: 1-{3-[4-(5-Fluoro-2-propoxy-phenyl)-piperazin-1-yl]-2-hydroxy-propyl)-piperidine-2,6-dione hydrochloride salt
IR (KBr): 1659.3 cm⁻¹; ¹H NMR (300 MHz, CDCl₃): δ 1.021-1.071 (t, 3H), 1.797-1.867 (m, 2H), 1.980-2.022 (t, 2H), 2.690-2.764 (q, 4H), 3.132-3.159 (t, 2H), 3.493(s, 6H), 3.763-3.825(q, 2H), 3.894-3.938(t,2H),4.010-4.077(m,2H),4.534-4.548(d,1H),6.657-6.791(m,3H); Mass (m/z): 408 (M⁺+1)

### Example 3

### Preparation of 2-{3-[4-(2-Cyclopentyloxy-5-fluoro-phenyl)-piperazin-1-yl]-2-oxo-propyl}-3a,4,7,7a-tetrahydro-isoindole-1,3-dione hydrochloride salt ( Compound No. 78)

### Step 1: Preparation of 2-{3-[4-(2-Cyclopentyloxy-5-fluoro-phenyl)-piperazin-1-yl]-2-oxo-propyl}-3a,4,7,7a-tetrahydro-isoindole-1,3-dione

To a clear solution of 2-{3-[4-(2-Cyclopentyloxy-5-fluoro-phenyl)-piperazin-1-yl]-2-hydroxy-propyl}-3a,4,7,7a-tetrahydro-isoindole-1,3-dione (1.0 gm, 0.00212 mol) in dichloromethane (20 mL) was added pyridinium chlorochromate (0.915 gm, 0.00425 mol) and the reaction mixture stirred at room temperature for about 2 hours and then refluxed further for about 4 hours. The reaction mixture was filtered through a celite pad and washed with dichloromethane. The combined filtrate was concentrated to yield the crude product, which was then purified on a column of silica gel (60-120 mesh) to yield 2-{3-[4-(2-Cyclopentyloxy-5-fluoro-phenyl)-piperazin-1-yl]-2-oxo-propyl}-3a,4,7,7a-tetrahydro-isoindole-1,3-dione. Yield: 0.3 gm (30%)

### Step 2: Preparation of 2-{3-[4-(2-Cyclopentyloxy-5-fluoro-phenyl)-piperazin-1-yl]-2-oxo-propyl}-3a,4,7,7a-tetrahydro-isoindole-1,3-dione hydrochloride salt

The hydrochloride salt of 1-{3-[4-(2-Cyclopentyloxy-5-fluoro-phenyl)-piperazin-1-yl]-2-hydroxy-propyl}-piperidine-2,6-dione was prepared following the previously disclosed procedure of Example 1, Step 5. Yield: 0.8 gm (80%)
IR (KBr): 1703.2 cm⁻¹; ¹H NMR (300 MHz, CDCl₃): δ 1.659-1.854 (m,8H), 2.255-2.301(d,2H),2.569-2.616 (d,2H), 3.223(s,2H),3.469-3.513 (d, 8H),4.458(s,1H), 4.529(s,2H),4.744 (s,1H),5.940 (s,2H), 6.654-6.794 (m,4H); Mass (m/z): 470 (M⁺+1)

The following compound was prepared following the above procedure
Compound No. 4: 2-{3-[4-(2-Isopropoxy-phenyl)-piperazin-1-yl]-2-oxo-propyl}-hexahydroisoindole-1,3-dione hydrochloride salt
IR (KBr): 1707 cm⁻¹; ¹H NMR (300 MHz, DMSO-d₆): δ 1.26-1.28 (d, 6H), 1.35-1.41 (m, 4H), 1.71-1.75 (m, 4H), 3.05 (m, 2H), 3.36-3.48 (m, 8H), 4.50 (brs, 2H), 4.57-4.64 (m, 2H), 6.90-6.96 (m, 4H), 10.70 (brs, 1H); Mass (m/z): 428.1 (M⁺+1)

### Example 4

### Preparation, of 2-{2-Fluoro-3-[4-(2-isopropoxy-phenyl)-piperazin-1-yl]-propyl}-3a,4,7,7a-tetrahydro-isoindole-1,3-dione hydrochloride salt (Compound No. 14)

### Step 1: Preparation of 2-{2-Fluoro-3-[4-(2-isopropoxy-phenyl)-piperazin-1-yl]-propyl}-3a,4,7,7a-tetrahydro-isoindole-1,3-dione

To a solution of 2-{2-Hydroxy-3-[4-(2-isopropoxy-phenyl)-piperazin-1-yl]-propyl}-3a,4,7,7a-tetrahydro-isoindole-1,3-dione (1 gm, 2.3 mmol) in dichloromethane was added diethyl amino sulfur trifluoride (0.754 g, 4.6 mmol). After completion of the reaction, water was added and the reaction mixture was extracted with dichloromethane. The organic layer was dried over anhydrous sodium sulfate and concentrated to yield the crude product. The compound was purified on silica gel column using dichloromethane-methanol to yield 2-{2-Fluoro-3-[4-(2-isopropoxy-phenyl)-piperazin-1-yl]-propyl}-3a,4,7,7a-tetrahydro-isoindole-1,3-dione. Yield: 0.410 gm (41%)

The following compounds were prepared similarly
Compound No. 35: 1-{2-Fluoro-3-[4-(4-fluorophenyl)piperazin-1-yl]-propyl}-piperidine-2,6-dione
Compound No. 36: 1-(2-Fluoro-3-{4-[2-(2,2,2-trifluoro-ethoxy)-phenyl]-piperazin-1-yl}propyl)-3,4-dimethylpyrrole-2,5-dione

### Step 2: Preparation of 2-{2-Fluoro-3-[4-(2-isopropoxy-phenyl)-piperazin-1-yl]-propyl}-3a,4,7,7a-tetrahydro-isoindole-1,3-dione hydrochloride salt

The hydrochloride salt of 2-{2-Fluoro-3-[4-(2-isopropoxy-phenyl)-piperazin-1-yl]-propyl}-3a,4,7,7a-tetrahydro-isoindole-1,3-dione was prepared following the previously disclosed procedure of Example 1, Step 5. Yield: 0.385 gm (89%)
IR (KBr): 1709.2 cm⁻¹; ¹H NMR (300 MHz, CDCl₃): δ 1.58 (d, 6H), 2.22-2.26 (m, 2H), 2.59-2.64 (m, 2H), 3.43-3.49 (m, 2H), 3.69-4.24 (m, 8H), 4.85-5.10 (m, 6H), 5.91 (brs, 2H), 7.01-7.43 (m, 3H), 8.18-8.19 (m, 1H); Mass (m/z): 430 (M⁺+1)

### Example 5

### Preparation of 2-{3-[4-(2-Cyclopentyloxy-5-fluoro-phenyl-piperazin-1-yl]-propyl}-5,6-dihydroxy-hexahydro-isoindole-1,3-dione hydrochloride salt (Compound No. 102)

### Step 1; Preparation of 2-{3-[4-(2-Cyclopentyloxy-5-fluoro-phenyl)-piperazin-1-yl]-propyl}-5,6-dihydroxy-hexahydro-isoindole-1,3-dione

To a clear solution of 2-{3-[4-(2-Cyclopentyloxy-5-fluoro-phenyl)-piperazin-1-yl]-propyl}-3a,4,7,7a-tetrahydro-isoindole-1,3-dione(1.0 gm, 0.002) in ethanol (20 mL) was added potassium permanganate solution (0.417 gm, 0.0026, in water 5 mL) dropwise at 0-5° C. The reaction mixture was stirred at room temperature for about 6-8 hours. After completion of the reaction, the reaction mixture was filtered through a celite pad and washed with ethanol. The filtrate was concentrated to yield the crude product, which was then purified by column chromatography to yield 2-{3-[4-(2-Cyclopentyloxy-5-fluoro-phenyl)-piperazin-1-yl]-propyl}-5,6-dihydroxy-hexahydro-isoindole-1,3-dione. Yield: 0.51 gm (47%)

### Step 2: Preparation of 2-{3-[4-(2-Cyclopentyloxy-5-fluoro-phenyl)-piperazin-1-yl]-propyl}-5,6-dihydroxy-hexahydro-isoindole-1,3-dione hydrochloride salt

The hydrochloride salt of 2-{3-[4-(2-Cyclopentyloxy-5-fluoro-phenyl)-piperazin-1-yl]-propyl}-5,6-dihydroxy-hexahydro-isoindole-1,3-dione was prepared following the previously disclosed procedure of Example 1, Step 5. Yield: 0.6 gm (56%)
IR (KBr): 1697.7 cm⁻¹; ¹H NMR (300 MHz, CDCl₃): δ 1.67-1.77 (m, 10H), 1.84-1.89 (t, 4H), 3.15 (s, 6H), 3.36 (s, 2H), 3.47-3.58 (m, 6H), 3.82 (s, 2H), 4.72 (s, 1H), 6.62-6.77 (m, 3H)

The following compounds were prepared by following the above procedure:
Compound No. 44: 2-{3-[4-(5-Fluoro-2-isopropoxy-phenyl)-piperazin-1-yl]-propyl}-5,6-dihydroxy-hexahydro-isoindole-1,3-dione hydrochloride salt
¹H NMR (300 MHz, CDCl₃): δ 1.11-1.37 (m, 6H), 1.84-1.94 (m, 6H), 3.05-3.13 (d, 6H), 3.47 (s, 6H), 3.79 (s, 2H), 4.06-4.08 (d, 1H), 4.49-4.51(1H,d), 4.72-4.75 (d, 3H), 6.62-6.71 (m, 3H); Mass (m/z): 464 (M⁺+1)
Compound No. 50: 5,6-Dihydroxy-2-{3-[4-(2-methoxy-5-methyl-phenyl)-piperazin-1-yl]-propyl}-hexahydro-isoindole-1,3-dione hydrochloride salt
IR (KBr): 1699 cm⁻¹; Mass (m/z): 432 (M⁺+1)
Compound No. 54: 2-{3-[4-(2-Cyclopentyloxy-phenyl)-piperazin-1-yl]-propyl}-5,6-dihydroxy-hexahydro-isoindole-1,3-dione hydrochloride salt
¹H NMR (300 MHz, CDCl₃): δ 1.263-1.885 (m, 8H), 2.137-2.177 (t, 4H), 3.077-3.131 (t,5H), 3.322-3.366 (t, 2H), 3.509-3.168 (m, 6H), 3.726-3.746 (t, 2H), 3.944 (s, 6H), 4.808-4.836 (m, 1H), 6.869-7.036 (m, 4H); Mass (m/z): 472 (M⁺+1)
Compound No. 76: 2-{3-[4-(2-Cyclopentyloxy-phenyl)-piperazin-1-yl]-2-oxo-propyl}-5,6-dihydroxy-hexahydro-isoindole-1,3-dione hydrochloride salt
IR (KBr): 1704.4 cm⁻¹; ¹H NMR (300 MHz, CDCl₃): δ 1.588-2.008 (m,8H), 2.162 (s,4H), 3.213 (s,3H), 3.446-3.516 (q,6H), 3.793 (s,4 H),4.616-4.732 (d,6H), 6.810-6.947(m,4H); Mass (m/z): 486 (M⁺+1)
Compound No. 104: 2-{3-[4-(5-Fluoro-2-propoxy-phenyl)-piperazin-1-yl]-propyl}-5,6-dihydroxy-hexahydro-isoindole-1,3-dione hydrochloride salt
IR (KBr): 1697.3 cm⁻¹; ¹H NMR (300 MHz, CDCl₃): δ 1.036-1.086 (t, 3 H), 1.185-1.255 (m, 4H), 1.812-1.880 (m, 4H), 1.944 (s, 2H), 3.030-3.061 (t, 4 H), 3.446-3.516 (m, 10H), 3.569-3.661(m, 1H), 3.906-3.927 (d, 2H), 4.080 (s, 1H), 6.601-6.774 (m, 3H); Mass (m/z): 464.58 (M⁺+1)
Compound No. 120: 2-{3-[4-(2-Cyclopentyloxy-5-fluoro-phenyl)-piperazin-1-yl]-2-oxo-propyl}-5,6-dihydroxy-hexahydro-isoindole-1,3-dione hydrochloride salt
IR (KBr): 1704.9 cm⁻¹; Mass (m/z): 504 (M⁺+1)
Compound No. 122: 2-{3-[4-(5-Fluoro-2-isopropoxy-phenyl)-piperazin-1-yl]-2-oxo-propyl}-5,6-dihydroxy-hexahydro-isoindole-1,3-dione hydrochloride salt
IR (KBr): 1706.4 cm⁻¹; Mass (m/z): 478(M⁺+1)
Compound No. 124: 2-{3-[4-(5-Fluoro-2-isopropoxy-phenyl)-piperazin-1-yl]-2-hydroxy-propyl}-5,6-dihydroxy-hexahydro-isoindole-1,3-dione hydrochloride salt
IR (KBr): 1699.3 cm⁻¹; Mass (m/z): 480 (M⁺+1)
Compound No. 126: 2-{3-[4-(2-Cyclopentyloxy-5-fluoro-phenyl)-piperazin-1-yl]-2-hydroxy-propyl}-5,6-dihydroxy-hexahydro-isoindole-1,3-dione hydrochloride salt
IR (KBr): 1699.1 cm⁻¹; Mass (m/z): 506 (M⁺+1)

### Example 6

### Preparation of 6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione hydrochloride salt (Compound No. 31)

### Step 1: Preparation of 6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione

To a solution 5,6-Dihydroxy-2-{3-[4-(2-isopropoxy-phenyl)-piperazin-1-yl]-propyl}-hexahydro-isoindole-1,3-dione (0.5 gm, 0.001 mol) (prepared according to example 5) in dichloromethane (5 mL) was added diethyl amino sulfur trifluoride (0.18 gm, 0.001 mol) dropwise at 0-5° C. and the reaction mixture was stirred for about 4 hrs. After completion of the reaction, the reaction mixture was quenched by adding water (15 mL). The reaction mixture was extracted with dichloromethane (2×10 mL), and the combined organic layers were dried over anhydrous sodium sulfate and concentrated. The crude product was purified on a column of silica gel (60-120 mesh) using dichloromethane:methanol as eluent to yield 6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione.

### Step 2: Preparation of 6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione hydrochloride salt

The hydrochloride salt of 6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione was prepared following the previously disclosed procedure of Example 1, Step 5. Yield: 0.5 gm (85%)
IR (KBr): 1700.1 cm⁻¹; ¹H NMR (300 MHz, CDCl₃): δ 1.37-1.38 (d, 6H), 2.18-2.54 (m, 6H), 3.02-3.08 (m, 4H), 3.29 (m, 2H), 3.45-3.69 (m, 8H), 4.61 (m, 1H), 5.03-5.08 (m, 1H), 5.28-5.31 (m, 1H), 6.87-7.24 (m, 4H), 12.42 (brs, 1H); Mass (m/z): 492.2 (M⁺+1)

### Example 7

### Preparation of 1-{3-[4-(5-Fluoro-2-methoxy-phenyl)-piperazin-1-yl]-propyl}-3,3,4-trimethyl-pyrrolidine-2,5-dione hydrochloride salt (Compound No. 168)

### Step 1: Preparation of 3-[4-(5-fluoro-2-methoxyphenyl)piperazin-1-yl]propionitrile

To a solution of 1-(5-fluoro-2-methoxy phenyl)piperazine (2.0 gm, 0.009 mol) in methanol (25 mL) was added acrylonitrile (1.0 gm, 0.018 mol) under stirring at room temperature. The reaction mixture was stirred for about 3-4 hours. After completion of the reaction, the reaction mixture was concentrated on buchi to yield 3-[4-(5-fluoro-2-methoxyphenyl)piperazin-1-yl]propionitrile. Yield: 2.2 gm (88%)

### Step 2: Preparation of 3-[4-(5-fluoro-2-methoxyphenyl)piperazin-1-yl]propylamine

To a solution of 3-[4-(5-fluoro-2-methoxyphenyl)piperazin-1-yl]propionitrile (2 gm, 0.0076 mol) in methanol/ammonia (20 mL) was added palladium/carbon (10%) w/w of the compound prepared in Example 7, Step 1 and the reaction mixture was hydrogenated at 55-60 psi for about 4-5 hours. The reaction mixture was then filtered through a celite pad, washed with methanol, and the filtrate was concentrated to yield 3-[4-(5-fluoro-2-methoxyphenyl)piperazin-1-yl]propylamine. Yield: 2.0 gm (99%)

### Step 3: Preparation of 1-{3-[4-(5-Fluoro-2-methoxy-phenyl)-piperazin-1-yl]-propyl)-3,3,4-trimethyl-pyrrole-2,5-dione

A solution of 3-[4-(5-fluoro-2-methoyphenyl)piperazin-1-yl]propylamine 1.0 gm, 0.0037 mol) and 3,3,4-trimethyldihydrofuran-2,5-dione (0.53 gm, 0.00376 mole) in toluene (15 mL) was refluxed for 1 hour. The reaction mixture was concentrated to yield the crude product, which was purified on a column of silica gel (100-120 mesh) using dichloromethane:methanol as eluent to yield 1-{3-[4-(5-Fluoro-2-methoxy-phenyl)-piperazin-1-yl]-propyl)-3,3,4-trimethyl-pyrrole-2,5-dione. Yield: 1.2 gm (82%)

### Step 4: Preparation of 1-{3-[4-(5-Fluoro-2-methoxy-phenyl)-piperazin-1-yl]-propyl)-3,3,4-trimethyl -pyrrole-2,5-dione hydrochloride salt

The hydrochloride salt of 1-{3-[4-(5-Fluoro-2-isopropoxy-phenyl)-piperazin-1-yl]-propyl)-3,3,4-trimethyl-pyrrole-2,5-dione was prepared following the previously disclosed procedure of Example 1, Step 5. Yield: 0.8 gm (85%)
IR (KBr)═ 1692.6 cm⁻¹; H¹ NMR (300 MHz, CDCl₃)δ: 1.163-1.253 (6H, m), 1.332-1.351 (3H, d), 2.239 (2H, s), 2.611-2.678 (1H, m), 3.167 (2H, s), 3.620-3.655 (6H, d), 3.997 (5H, s), 4.633 (2H, s), 6.946-7.696 (3H, m); Mass (m/z)=392 (M⁺+1)

### Example 8

### Preparation of 1-{3-[4-(5-Fluoro-2-isopropoxy-phenyl)-piperazin-1-yl]-propyl}-3-methylene-pyrrolidine-2,5-dione hydrochloride salt (Compound No. 136)

### Step 1: Preparation of 1-{3-[4-(5-Fluoro-2-isopropoxy-phenyl)-piperazin-1-yl]-propyl}-3-methylene-pyrrolidine-2,5-dione

A solution of 3-[4-(5-fluoro-2-isopropoxyphenyl)piperazin-1-yl]propyl amine (1.0 gm, 0.0034 mol) and itaconic anhydride (0.38 gm, 0.0034 mole) in toluene (15 mL) was refluxed for 1 hour. The reaction mixture was concentrated to form a crude residue, which was purified by column chromatography to yield 1-{3-[4-(5-Fluoro-2-isopropoxy-phenyl)-piperazin-1-yl]-propyl}-3-methylene-pyrrolidine-2,5-dione. Yield: 0.700 gm (54%)

### Step 2: Preparation of 1-{3-[4-(5-Fluoro-2-isopropoxy-phenyl)-piperazin-1-yl]-propyl}-3-methylene-pyrrolidine-2,5-dione hydrochloride salt

The hydrochloride salt of 1-{3-[4-(5-Fluoro-2-isopropoxy-phenyl)-piperazin-1-yl]-propyl}-3-methylene-pyrrolidine-2,5-dione was prepared following the previously disclosed procedure of Example 1, Step 5. Yield: 0.5 gm (90%)
H¹ NMR (300 MHz, CDCl₃)δ: 1.23-1.25 (6H, d), 2.10 (2H, s), 2.27-2.34 (2H, q), 3.03-3.08 (4H, t), 3.47-3.68 (8H, m), 4.48-4.52 (1H, t), 6.36 (2H, s), 6.62-6.80 (3H, m); Mass (m/z)=390(M⁺+1)

The following compounds were prepared following the above procedure:
Compound No. 134: 1-{3-[4-(3-Fluoro-2-isopropoxy-phenyl)-piperazin-1-yl]-propyl}-3-methylene-pyrrolidine-2,5-dione hydrochloride salt
IR (KBr): 1711.5 cm⁻¹; ¹H NMR (300 MHz, CDCl₃)δ: 1.185-1.364 (6H, m), 2.009-2.099 (2H, m), 2.188 (2H, s), 2.940 (3H, s), 3.130 (3H, s), 3.374-3.721 (7H, m), 4.461-4.521 (1H, q), 6.352-6.357 (1H, s), 6.668-6.984 (3H, m); Mass (m/z)=390.7 (M⁺+1)
Compound No. 162: 3-Methylene-1-[3-(4-O-tolyl-piperazin-1-yl)-propyl]-pyrrolidine-2,5-dione hydrochloride salt
Compound No. 166: 1-{3-[4-(2-Cyclopentyloxy-phenyl)-piperazin-1-yl]-propyl}-3-methylene-pyrrolidine-2,5-dione hydrochloride salt
IR KBr): 1706.7 cm⁻¹; Mass (m/z): 398 (M⁺+1)

### Example 9

### Preparation of 1-{3-[4-(2-Methoxy-phenyl)-piperazin-1-yl]-propyl)-3-methyl-4-(1-phenyl-ethylamino)-pyrrolidine-2,5-dione hydrochloride salt( Compound No. 140)

### Step 1: Preparation of 1-{3-[4-(2-Methoxy-phenyl)-piperazin-1-yl]-propyl)-3-methyl-4-(1-phenyl-ethylamino)-pyrrolidine-2,5-dione

To a solution of 1-{3-[4-(2-Methoxy-phenyl)-piperazin-1-yl]-propyl)-3-methyl-pyrrole-2,5-dione (0.5 gm, 0.001 mol) in methanol was added an equimolar quantity of 1-phenylethyl amine 0.21 gm, 0.0017 mol) and the reaction mixture stirred at room temperature for about 10-12 hours. The reaction mixture was concentrated to yield the crude product, which was purified on a column of silica gel (100-120 mesh) using dichloromethane:methanol as eluent to yield 1-{3-[4-(2-Methoxy-phenyl)-piperazin-1-yl]-propyl)-3-methyl-4-(1-phenyl-ethylamino)-pyrrolidine-2,5-dione. Yield: 0.6 gm (89%)

### Step 2: Preparation of 1-{3-[4-(2-Methoxy-phenyl)-piperazin-1-yl]-propyl)-3-methyl-4-(1-phenyl-ethylamino)-pyrrolidine-2,5-dione hydrochloride salt

The hydrochloride salt of 1-{3-[4-(2-Methoxy-phenyl)-piperazin-1-yl]-propyl)-3-methyl-4-(1-phenyl-ethylamino)-pyrrolidine-2,5-dione was prepared following the previously disclosed procedure of Example 1, Step 5. Yield: 0.5 gm (85%)
IR (DCM): 1690.8 cm⁻¹; Mass (m/z): 465 (M⁺+1)

The following compounds were prepared following the above procedure:
Compound No. 148: 3-Cyclopropylamino-1-{3-[4-(2-ethoxy-phenyl)-piperazin-1-yl]-propyl}-pyrrolidine-2,5-dione hydrochloride salt
IR (KBr): 1707.3 cm⁻¹; Mass (m/z): 401 (M⁺+1)
Compound No. 152: 1-{3-[4-(2-Cyclopentyloxy-phenyl)-piperazin-1-yl]-propyl)-3-cyclopropylamino-pyrrolidine-2,5-dione hydrochloride salt
IR (KBr): 1713.3 cm⁻¹; Mass (m/z): 441(M⁺+1)
Compound No. 164: 1-{3-[4-(2-Methoxy-phenyl)-piperazin-1-yl]-propyl)-3-methyl-4-[(thiophen-2-ylmethyl)-amino]-pyrrolidine-2,5-dione hydrochloride salt
IR (KBr): 1701.5 cm⁻¹; Mass (m/z): 457 (M⁺+1)

### Example 10

### Preparation of 1-{3-[4-(2-Methoxy-5-methyl-phenyl)-piperazin-1-yl]-propyl}-piperidine-2,6-dione hydrochloride salt (Compound No. 48)

### Step 1: Preparation of 1-(3-bromopropyl)-piperidine-2,6-dione

A mixture of piperidine-2,6-dione (2 gm, 0.017 mole), 1,3-dibromopropane (5.3 gm, 0.026 mole), potassium carbonate (4.88 gm, 0.035 mole) and tetrabutylammonium iodide 0.13 gm, 0.0035 mole) in acetone (20 mL) was stirred at 40° C. for about 8 hours. Inorganics were filtered and washed with acetone; the solvent was removed from the filtrate under pressure; and the resulting residue was suspended in water. The aqueous solution (suspension) was extracted with ethyl acetate. The organic layer was washed with water, dried over anhydrous sodium sulfate and evaporated in vacuo to form the crude product. The crude product was purified on silica gel (60-120 mesh) column using dichloromethane as eluent to yield 1-(3-bromopropyl)-piperidine-2,6-dione. Yield: 3.1 gm (76%)

### Step 2: Preparation of 1-(3-{4-[2-(2-methoxy-5-methyl)-phenyl]-piperazin-1-yl}propyl)-piperidine-2,6-dione

A mixture of 1-(3-bromopropyl)-piperidine-2,6-dione (2 gm, 0.0085 mole), anhydrous potassium carbonate (2.36 gm, 0.0017 mol) and 2-methoxy-5-methyl phenyl piperazine (1.76 gm, 6.0085 mole) in dimethylformamide (20 mL) was heated to and maintained at 75-78° C. for about 6-8 hours. The reaction mixture was quenched by adding water (60 mL), extracted with ethyl acetate, concentrated and purified on silica gel (60-120 mesh) column using dichloromethane and methanol as eluent to yield 1-(3-{4-[2-(2-methoxy-5-methyl)-phenyl]-piperazin-1-yl}propyl)-piperidine-2,6-dione. Yield: 2.2 gm (72%)

### Step 3: Preparation of 1-(3-{4-[2-(2-methoxy-5-methyl)-phenyl]-piperazin-1-yl}propyl)-piperidine-2,6-dione hydrochloride salt

The hydrochloride salt of 1-(3-{4-[2-(2-methoxy-5-methyl)-phenyl]-piperazin-1-yl}propyl)-piperidine-2,6-dione was prepared following the previously disclosed procedure of Example 1, Step 5. Yield: 0.6 gm (87%)
IR (KBr): 1668.8 cm⁻¹; Mass (m/z): 360 (M⁺+1)

The following compounds were similarly prepared using the above procedure:
Compound No. 52: 1-(3-{4-[5-Fluoro-2-(2,2,2-trifluoro-ethoxy)-phenyl]-piperazin-1-yl}-propyl)-piperidine-2,6-dione hydrochloride salt
IR (KBr): 1669.9 cm⁻¹; Mass (m/z): 432 (M⁺+1)
Compound No. 98: 1-{3-[4-(5-Fluoro-2-propoxy-phenyl)-piperazin-1-yl]-propyl}-piperidine-2,6-dione hydrochloride salt
IR (KBr)═ 1671.8 cm⁻¹; ¹H NMR (300 MHz, CDCl₃)δ: 1.02-1.07 (3H, t), 1.96-2.00 (2H, t), 2.16-2.21 (2H, t), 2.68-2.72 (6H, t), 3.02-3.07 (6H, t), 3.52 (6H, s), 3.88-3.94 (4H, m), 6.63-6.79 (3H, m); Mass (m/z)=392 (M⁺+1)
Compound No. 128: 1-{3-[4-(2-Cyclopentyloxy-5-fluoro-phenyl)-piperazin-1-yl]-propyl}-piperidine-2,6-dione hydrochloride salt
IR (KBr)═ 1673.9 cm⁻¹; ¹H NMR (300 MHz, CDCl₃)≡: 1.69-2.01 (10H, m), 2.19 (2H, s), 2.68-2.72 (4H, t), 3.02-3.05 (4H, d), 3.51 (6H, s), 3.88-3.92 (2H, t), 4.75 (1H, s), 6.61-6.79 (3H, m); Mass (m/z)=418 (M⁺+1)
Compound No. 130: 1-{3-[4-(3-Fluoro-2-isopropoxy-phenyl)-piperazin-1-yl]-propyl}-piperidine-2,6-dione hydrochloride salt
IR (KBr)═ 1698.8 cm⁻¹; ¹H NMR (300 MHz, CDCl₃)δ: 1.20-1.30 (6H, dd), 1.99-2.01 (2H, d), 2.21 (2H, s), 2.68-2.72 (4H, t), 2.92-3.07 (4H, d), 3.47-3.59 (6H, t), 3.90 (2H, s), 4.44-4.50 (1H, m), 6.68-6.99 (3H, m); Mass (m/z)=392.8 (M⁺+1)
Compound No. 132: 1-{3-[4-(3-Fluoro-2-methoxy-phenyl)-piperazin-1-yl]-propyl}-piperidine-2,6-dione hydrochloride salt
IR (KBr): 1669.6 cm⁻¹; ¹H NMR (300 MHz, CDCl₃)δ: 1.96-2.03 (2H, q), 2.17-2.24 (2H, q), 2.68-2.72 (4H, t), 3.03-3.09 (4H, t), 3.55 (6H, s), 3.88-3.90 (5H, d), 6.68-6.96 (3H, m); Mass (m/z)=364 (M⁺+1)
Compound No. 138: 1-{3-[4-(5-Fluoro-2-(2,2,3,3-tetrafluoro-propoxy)-phenyl]-piperazin-1-yl}-propyl)-piperidine-2,6-dione hydrochloride salt
IR (KBr): 1688.7 cm⁻¹; Mass (m/z): 464 (M⁺+1)
Compound No. 142: 1-{3-[4-(5-Fluoro-2-trifluoromethoxy-phenyl)-piperazin-1-yl]-propyl)-piperidine-2,6-dione hydrochloride salt
IR (KBr): 1703 cm⁻¹; Mass (m/z): 400 (M⁺+1)
Compound No. 170: 1-{3-[4-(2-Cyclopentyloxy-phenyl)-piperazin-1-yl]-propyl}-piperidine-2,6-dione hydrochloride salt
IR (KBr): 1673.1 cm⁻¹; H NMR (300 MHz, CDCl₃)δ: 1.631-1.730 (10H, m), 2.010 (2H, s), 2.683-2.726 (4H, t), 3.014-3.041 (4H, d), 3.494 (6H, s), 3.882-3.925 (2H, t), 4.799-4.817 (1H, t), 6.841-7.014 (4H, m); Mass (m/z)=400 (M⁺+1)

### Example 11

### Preparation of 3-{3-[4-(5-Fluoro-2-isopropoxy-phenyl)-piperazin-1-yl]-propyl}-1-methyl-3-aza-bicyclo [3.1.0]hexane-2.4-dione hydrochloride salt (Compound No. 64)

### Step 1: Preparation of 3-{3-[4-(5-Fluoro-2-isopropoxy-phenyl)-piperazin-1-yl]-propyl}-1-methyl-3-aza-bicyclo[3.1.0]hexane-2,4-dione

To a suspension of sodium hydride (0.037 gm, 0.0015 mol) in dimethylsulphoxide (15 mL) was added trimethyl sulphoxonium iodide (0.34 gm, 0.0015 mol) in lots at room temperature. A solution of 1-{3-[4-(5-Fluoro-2-isopropoxy-phenyl)-piperazin-1-yl]-propyl)-3-methyl-pyrrole-2,5-dione (0.5 gm, 0.0013 mol) in dimethylsulphoxide (5 mL) was then added to the reaction mixture at 10-15° C. and the reaction mixture was stirred for about 10-15 minutes. The reaction mixture was quenched by adding water (30 mL) and extracted with ethyl acetate; and the combined organic layers were concentrated to yield the crude product, which was then purified by column chromatography to yield 3-{3-[4-(5-Fluoro-2-isopropoxy-phenyl)-piperazin-1-yl]-propyl}-1-methyl-3-aza-bicyclo[3.1.0]hexane-2,4-dione. Yield: 0.25 gm (48%)

### Step 2: Preparation of 3-{3-[4-(5-Fluoro-2-isopropoxy-phenyl)-piperazin-1-yl]-propyl}-1-methyl-3-aza-bicyclo[3.1.0]hexane-2,4-dione hydrochloride salt

The hydrochloride salt of 3-{3-[4-(5-Fluoro-2-isopropoxy-phenyl)-piperazin-1-yl]-propyl}-1-methyl-3-aza-bicyclo[3.1.0]hexane-2,4-dione was prepared following the previously disclosed procedure of Example 1, Step 5. Yield: 0.190 gm (37%)
IR (KBr): 1704 cm⁻¹; Mass (m/z): 404 (M⁺+1)

The following compounds were prepared by following above procedure:
Compound No. 56: 3-{3-[4-(3-Fluoro-2-methoxy-phenyl)-piperazin-1-yl]-propyl)-1-methyl-3-aza-bicyclo[3.1.0]hexane-2,4-dione hydrochloride salt
IR (KBr): 1613.8 cm⁻¹; Mass (m/z): 376 (M⁺+1)
Compound No. 58: 3-{3-[4-(5-Fluoro-2-methoxy-phenyl)-piperazin-1-yl]-propyl}-1-methyl-3-aza-bicyclo[3.1.0]hexane-2,4-dione hydrochloride salt
IR (KBr): 1650.3 cm⁻¹; Mass (m/z): 376 (M⁺+1)
Compound No. 60: 3-{3-[4-(2-Cyclopentyloxy-phenyl)-piperazin-1-yl]-propyl}-1-methyl-3-aza-bicyclo[3.1.0]hexane-2,4-dione hydrochloride salt
IR (KBr): 1617 cm⁻¹; Mass (m/z): 412 (M⁺+1)

### Example 12

### Preparation of 2-{3-[4-(2-Cyclopentyloxy-5-fluoro-phenyl)-piperazin-1-yl]-propyl}-5-fluoro-6-hydroxy-hexahydro-isoindole-1,3-dione hydrochloride salt (Compound No. 108)

### Step 1: Preparation of 4-(3-chloropropyl)tetrahydro-1aH-oxireno[f]isoindole-3,5(2H,4H)-dione

To a solution of 2-(3-chloropropyl)-3a,4,7,7a-tetrahydroisoindole-1,3-dione (1.0 gm, 0.0037 mole) in dichloromethane (10 mL) was added an equimolar quantity of metachloroperbenzoic acid (1.33 gm of 50%, 0.0037 mol) in dichloromethane at 0-5° C. The reaction mixture was stirred for about 6-8 hours. The reaction mixture was then poured into an ice-cold potassium carbonate solution (5%) and concentrated to yield 4-(3-chloropropyl)tetrahydro-1aH-oxireno[f]isoindole-3,5(2H,4H)-dione. Yield: 0.8 gm, 75%

### Step 2: Preparation of 4-{3-[4-(2-cyclopentyloxy-5-fluoro-phenyl)-piperazin-1-yl]-propyl}-hexahydro-1-oxa-4-aza-cyclopropa[f]indene-3,5-dione

A suspension of 4-(3-chloropropyl)tetrahydro-1aH-oxireno[f]isoindole-3,5 (2H,4H)-dione (0.5 gm, 0.002 mol), 1-(5-fluoro-2-cyclopentyloxyphenyl)piperazine (0.49 gm, 0.0018 mol), anhydrous potassium carbonate (0.567 gm, 0.004 mol) and potassium iodide (0.007 gm, 0.00004 mole) in dimethylformamide (20 mL) was heated at 50-55° C. for about 24 hours. The reaction was quenched by adding water and extracted with ethyl acetate; the combined organic layers were then dried over anhydrous sodium sulfate and concentrated to yield the crude product. The crude product was then purified on a column of silica gel (60-120 mesh) using dichloromethane:methanol as eluent to yield 4-{3-[4-(2-cyclopentyloxy-5-fluoro-phenyl)-piperazin-1-yl]-propyl}-hexahydro-1-oxa-4-aza-cyclopropa[f]indene-3,5-dione. Yield: 0.6 gm, 62%

### Step 3: Preparation of 2-{3-[4-(2-Cyclopentyloxy-5-fluoro-phenyl)-piperazin-1-yl]-propyl}-5-fluoro-6-hydroxy-hexahydro-isoindole-1,3-dione

To a solution of 4-{3-[4-(2-cyclopentyloxy-5-fluoro-phenyl)-piperazin-1-yl]-propyl}-hexahydro-1-oxa-4-aza-cyclopropa[f]indene-3,5-dione (0.5 gm, 0.001 mol) in dichloromethane (15 mL) was added diethyl amino sulfur trifluoride (0.26 gm, 0.0016 mol) dropwise under stirring at 0-5° C. The reaction mixture was further stirred at room temperature for about 2-3 hours. After the completion of the reaction, the reaction mixture was quenched by adding a dilute solution of sodium bicarbonate and extracted with dichloromethane; the combined organic layers were concentrated to yield the crude product, which was then purified on a column of silica gel (60-120 mesh) using dichloromethane:methanol as eluent to yield 2-{3-[4-(2-Cyclopentyloxy-5-fluoro-phenyl)-piperazin-1-yl]-propyl}-5-fluoro-6-hydroxy-hexahydro-isoindole-1,3-dione.

### Step 4: Preparation of 2-{3-[4-(2-Cyclopentyloxy-5-fluoro-phenyl)-piperazin-1-yl]-propyl}-5-fluoro-6-hydroxy-hexahydro-isoindole-1,3-dione hydrochloride salt

The hydrochloride salt of 2-{3-[4-(2-Cyclopentyloxy-5-fluoro-phenyl)-piperazin-1-yl]-propyl}-5-fluoro-6-hydroxy-hexahydro-isoindole-1,3-dione was prepared following the previously disclosed procedure of Example 1, Step 5. Yield: 0.100 gm (19%)
IR (KBr): 1638 cm⁻¹; Mass (m/z): 492 (M⁺+1)

The following compounds were prepared by following the above procedure:
Compound No. 66: 5-Fluoro-6-hydroxy-2-{3-[4-(2-isopropoxy-phenyl)-piperazin-1-yl]-propyl}-hexahydro-isoindole-1,3-dione hydrochloride salt
IR (KBr): 1613 cm⁻¹; Mass (m/z): 448 (M⁺+1)
Compound No. 82: 5-Fluoro-2-{3-[4-(5-fluoro-2-methoxy-phenyl)-piperazin-1-yl]-propyl}-6-hydroxy-hexahydro-isoindole-1,3-dione hydrochloride salt
IR (KBr): 1687 cm⁻¹; Mass (m/z): 438 (M⁺+1)
Compound No. 86: 5-Fluoro-2-{3-[4-(5-fluoro-2-isopropoxy-phenyl)-piperazin-1-yl]-propyl}-6-hydroxy-hexahydro-isoindole-1,3-dione hydrochloride salt
IR (KBr): 1709 cm⁻¹; Mass (m/z): 467 (M⁺+1)
Compound No. 106: 2-{3-[4-(2-Cyclopentyloxy-phenyl)-piperazin-1-yl]-propyl}-5-fluoro-6-hydroxy-hexahydro-isoindole-1,3-dione hydrochloride salt
IR (KBr): 1703 cm⁻¹; Mass (m/z): 474 (M⁺+1)

### Example 13

### Preparation of 2-{3-[4-(2-Cyclopentyloxy-5-fluoro-phenyl)-piperazin-1-yl]-propyl}-5-hydroxy-hexahydro-isoindole-1,3-dione hydrochloride salt (Compound No. 72)

### Step 1: Preparation of 2-{3-[4-(2-Cyclopentyloxy-5-fluoro-phenyl)-piperazin-1-yl]-propyl}-5-hydroxy-hexahydro-isoindole-1,3-dione

To a solution of 4-{3-[4-(2-cyclopentyloxy-5-fluoro-phenyl)-piperazin-1-yl]-propyl}-hexahydro-1-oxa-4-aza-cyclopropa[f]indene-3,5-dione (1.0 gm, 0.002 mol) in methanol (20 mL) was added palladium/carbon (0.5 gm) and the resulting reaction mixture was hydrogenated at 55-60° C. psi for about 24 hours. After completion of the reaction, the reaction mixture was filtered through a celite pad and washed with methanol; the combined filtrate was concentrated to yield the crude product, which was purified by column chromatography to yield 2-{3-[4-(2-Cyclopentyloxy-5-fluoro-phenyl)-piperazin-1-yl]-propyl}-5-hydroxy-hexahydro-isoindole-1,3-dione.

### Step 2: Preparation of 2-{3-[4-(2-Cyclopentyloxy-5-fluoro-phenyl)-piperazin-1-yl]-propyl}-5-hydroxy-hexahydro-isoindole-1,3-dione hydrochloride salt

The hydrochloride salt of 2-{3-[4-(2-Cyclopentyloxy-5-fluoro-phenyl)-piperazin-1-yl]-propyl}-5-hydroxy-hexahydro-isoindole-1,3-dione was prepared following the previously disclosed procedure of Example 1, Step 5. Yield: 0.200 gm, 20%
IR (KBr): 1696.9 cm⁻¹; ¹H NMR (300 MHz, CDCl₃): δ 1.69-1.92(m, 12H), 2.18-2.22 (m, 4H), 2.91-2.92 (d, 4H), 3.47-3.67 (m, 11H), 4.17 (s, 1H), 4.72-4.75 (q, 1H), 6.59-6.76 (m, 3H); Mass (m/z): 474 (M⁺+1)

The following compounds were prepared by following the above procedure:
Compound No. 70: 5-Hydroxy-2-{3-[4-(2-methoxy-phenyl)-piperazin-1-yl]-propyl}-hexahydro-isoindole-1,3-dione hydrochloride salt
IR (KBr): 1697.3 cm⁻¹; ¹H NMR (300 MHz, CDCl₃): δ 1.21-2.37 (m, 13H), 2.92 (s, 2H), 3.33 (s, 1H), 3.47-3.69 (m, 7H), 3.93 (s, 3H), 4.20 (s, 1H), 6.93-7.26 (m, 4H); Mass (m/z): 402 (M⁺+1)
Compound No. 74: 2-{3-[4-(2-Cyclopentyloxy-phenyl)-piperazin-1-yl]-propyl}-5-hydroxy-hexahydro-isoindole-1,3-dione hydrochloride salt
IR (KBr): 1693.7 cm⁻¹; ¹H NMR (300 MHz, CDCl₃): δ 1.66-2.17(m, 16H), 2.90-2.91 (d, 4H), 3.09 (s, 4H), 3.36 (s, 4H),3.55-3.65 (dd,3H), 4.12 (s, 1H), 4.80-4.82 (t, 1H), 6,83-7.00 (m, 4H); Mass (m/z): 456 (M⁺+1)
Compound No. 84: 2-{3-[4-(5-Fluoro-2-isopropoxy-phenyl)-piperazin-1-yl]-propyl}-5-hydroxy-hexahydro-isoindole-1,3-dione hydrochloride salt
IR (KBr): 1706 cm⁻¹; Mass (m/z):448(M⁺+1)

### Example 14

### Preparation of 4-Hydroxy-2-{3-[4-(2-methoxy-phenyl)-piperazin-1-yl]-propyl-3a,4,7,7a-tetrahydro-isoindole-1.3-dione hydrochloride salt (Compound No. 18)

### Step 1: Preparation of 4-Hydroxy-2-{3-[4-(2-methoxy-phenyl)-piperazin-1-yl]-propyl}-3a,4,7,7a-tetrahydro-isoindole-1,3-dione

A solution of 1-{3-[4-(2-methoxyphenyl)piperazin-1-yl]propyl}pyrrole-2,5-dione (1 gm, 0.003 mol (prepared as in Example 7) and 1-acetoxy-1,3-butadiene (0.34 gm, 0.003 mol) in toluene was refluxed for about 3-4 hours. The reaction mixture was concentrated under vacuum and to the thick residue thus obtained was added a mixture of methanol/hydrochloric acid (5 N, 20 mL) at 10-15° C. The reaction mixture was then stirred for about 4-6 hours. Solid sodium bicarbonate was added in lots until the reaction mixture was neutralized. Inorganics were filtered through a celite pad, washed with methanol and concentrated to yield the crude product. The crude product was purified on silica gel (60-120 mesh) column using dichloromethane:methanol as eluent to yield 4-Hydroxy-2-{3-[4-(2-methoxy-phenyl)-piperazin-1-yl]-propyl}-3a,4,7,7a-tetrahydro-isoindole-1,3-dione. Yield : 0.8 gm (88%)

### Step 2: Preparation of 4-Hydroxy-2-{3-[4-(2-methoxy-phenyl)-piperazin-1-yl]-propyl}-3a,4,7,7a-tetrahydro-isoindole-1,3-dione hydrochloride salt

The hydrochloride salt of 4-Hydroxy-2-{3-[4-(2-methoxy-phenyl)-piperazin-1-yl]-propyl}-3a,4,7,7a-tetraydro-isoindole-1,3-dione was prepared following the previously disclosed procedure of Example 1, Step 5. Yield: 0.6 gm (75%)
IR (KBr) cm⁻¹: 1693.9: ¹H NMR (300 MHz, CDCl₃):δ 2.04 (2H, m), 2.34-2.49 (2H, m), 3.07-3.18 (2H, m), 3.31 (2H,m),3.35-3.65 (10H,m),3.82-3.86 (4H,brs), 6.00-6.05 (2H,d), 6.86-7.20(4H,m), 12.80(1H,brs); Mass (m/z): 400.4 (M⁺+1)

The following compounds were prepared by following above procedure:
Compound No. 16: Acetic acid 2-{3-[4-(2-methoxy-phenyl)-piperazin-1-yl]-propyl}-1,3-dioxo-2,3,3a,4,7,7a-hexahydro-1H-inden-4-yl ester hydrochloride salt
IR (KBr): 1736.9, 1696.8 cm⁻¹; ¹H NMR (300 MHz, CDCl₃): δ 2.09 (s, 3H), 2.27-2.39 (m, 3H), 2.67-2.72 (d, 1H), 3.07-3.20 (m, 5H), 3.53-3.65 (m, 9H), 3.89 (s, 3H), 5.41 (brs, 1H), 6.06 (brs, 2H), 6.89-6.94 (m, 2H), 7.09-7.11 (m, 2H), 12.83 (brs, 1H); Mass (m/z): 442.4 (M⁺+1)
Compound No. 92: Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester hydrochloride salt
IR (KBr): 1700.8 cm⁻¹; ¹H NMR (300 MHz, CDCl₃): δ 1.48-1.50 (d, 6H), 2.06 (s, 3H), 3.12 (s, 2H), 3.65 (s, 8H), 3.96 (s, 2H), 4.43 (s, 2H), 4.67 (s, 1H), 5.42 (s, 2H), 6.19 (s, 2H), 6.69-7.61 (m, 3H); Mass (m/z): 546 (M⁺+1).
Compound No. 94: Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester hydrochloride salt
¹H NMR (300 MHz, CDCl₃): δ 1.260-1.494 (8H, m), 2.130 (6H, s), 2.213-2.226 (2H, d), 3.136 (2H,s), 3.655 (8H,m),3.938 (2H, s), 4.391 (2H,s), 4.675(1H,s), 5.428(2H,s), 6.190(2H,s),6.961-7.600(3H,m); Mass (m/z): 572 (M⁺+1)
Compound No. 96: 2-{3-[4-(5-Fluoro-2-isopropoxy-phenyl)-piperazin-1-yl]-propyl}-4,7-dihydroxy-3a,4,7,7a-tetrahydro-isoindole-1,3-dione hydrochloride salt
¹H NMR (300 MHz, CDCl₃): δ 1.19-1.33 (6H, m), 1.70 (2H, s), 2.37 (2H, s), 3.01-3.18 (6H,d), 3.47-3.52 (6H,t),3.72 (2H, s), 4.47-4.49 (1H,d), 4. 75(2H,s), 6.48(2H,s), 6.61-6.80 (3H,m); Mass (m/z): 464 (M⁺+1)
Compound No. 118: 2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione hydrochloride salt
¹H NMR (300 MHz, CDCl₃): δ 1.25-1.43 (8H, m), 2.31-2.34 (2H, d), 3.06-3.73 (14H, m), 4.49 (1H,s), 4.74 (2H,s),6.49 (2H, s), 6.49-6.80 (3H,m).
Compound No. 144: Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-yl ester hydrochloride salt
IR (KBr): 1731.9 cm⁻¹; Mass (m/z): 514 (M⁺+1)
Compound No. 146: 2-{3-[4-(2-Ethoxy-phenyl)-piperazin-1-yl]-propyl)-4,7-dihydroxy-3a,4,7,7a-tetrahydro-isoindole-1,3-dione hydrochloride salt
IR (KBr): 1704.3 cm⁻¹; Mass (m/z): 430 (M⁺+1)
Compound No. 150: Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester hydrochloride salt
IR (KBr): 1693 cm⁻¹; Mass (m/z): 500 (M⁺+1)
Compound No. 154: 4,7-Dihydroxy-2-{3-[4-(2-methoxy-phenyl)-piperazin-1-yl]-propyl)-3a,4,7,7a-tetrahydro-isoindole-1,3-dione hydrochloride salt
IR (KBr): 1695.8 cm⁻¹; Mass (m/z): 416 (M⁺+1)
Compound No. 156: Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester hydrochloride salt
IR (KBr): 1704 cm⁻¹; Mass (m/z): 554 (M⁺+1)
Compound No. 160: 2-{3-[4-(2-Cyclopentyloxy-phenyl)-piperazin-1-yl]-propyl}-4,7-dihydroxy-3a,4,7,7a-tetrahydro-isoindole-1,3-dione hydrochloride salt
IR (KBr): 1713 cm⁻¹; Mass (m/z): 470 (M⁺+1)

### Example 15

### Preparation of 1-{3-[4-(2-Cyclopentyloxy-phenyl)-piperazin-1-yl]-2-hydroxy-propyl}-3-cyclopropylamino-4-methyl-pyrrolidine-2,5-dione hydrochloride salt ( Compound No. 38)

### Step 1: Preparation of 2-{3-[4-(2-cyclopentyloxy-phenyl)-piperazine-1-yl]-2-hydroxy-propyl}-isoindole-1,3-dione

A mixture of 2-oxiranylmethyl-isoindole-1,3-dione (2.0 gm, 0.0098 mol) (prepared as in Example 1) and 2-cyclopentyloxyphenyl piperazine (2.6 gm, 0.0098 mol) in alcohol (20 mL) was refluxed for about 4-5 hours. The reaction mixture was concentrated on buchi and the resulting residue was purified by column chromatography to yield 2-{3-[4-(2-cyclopentyloxy-phenyl)-piperazine-1-yl]-2-hydroxy-propyl}-isoindole-1,3-dione. Yield: 4.0 gm (86%)

### Step 2: Preparation of 1-amino-3-[4-(2-cyclopentyloxy-phenyl)-piperazin-1-yl}-propan-2-ol

To a solution of 2-{3-[4-(2-cyclopentyloxy-phenyl)-piperazine-1-yl]-2-hydroxy-propyl}-isoindole-1,3-dione (1.0 g, 0.0022 mol) in alcohol (15 mL) was added hydrazine hydrate (0.134 g, 0.0026 mol) and the reaction mixture refluxed for about 1 hour. The reaction mixture was cooled; a solid that precipitated was filtered, washed with chilled alcohol; the filtrate thus obtained was concentrated to yield 1-amino-3-[4-(2-cyclopentyloxy-phenyl)-piperazin-1-yl}-propan-2-ol. Yield: 0.64 gm (90%)

### Step 3: Preparation of 1-{3-[4-(2-cyclopentyloxy-phenyl)-piperazin-1-yl]-2-hydroxy-propyl}-3-methyl-pyrrole-2,5-dione

A mixture of 1-amino-3-[4-(2-cyclopentyloxy-phenyl)-piperazin-1-yl}-propan-2-ol (0.5 gm, 0.0016 mol) and citaconic anhydride (0.18 gm, 0.0016 mol) in toluene was refluxed for about 1 hour. The reaction mixture was concentrated on buchi and a resulting thick residue thus obtained was purified by column chromatography to yield 1-{3-[4-(2-cyclopentyloxy-phenyl)-piperazin-1-yl]-2-hydroxy-propyl}-3-methyl-pyrrole-2,5-dione. Yield: 0.52 gm (80%)

### Step 4: Preparation of 1-{3-[4-(2-cyclopentyloxy-phenyl)-piperazin-1-yl]-2-hydroxy-propyl}-3-cyclopropylamino-4-methyl-pyrrolidine-2,5-dione

To a solution of 1-{3-[4-(2-cyclopentyloxy-phenyl)-piperazin-1-yl]-2-hydroxy-propyl}-3-methyl-pyrrole-2,5-dione (0.5 gm, 0.0012 mol) in methanol (15 mL) was added cyclopropylamine (0.083 gm, 0.0015 mol) and the reaction mixture was stirred at room temperature for about 10-12 hours. The reaction mixture was concentrated and the resulting residue was concentrated and purified by column chromatography to yield 1-{3-[4-(2-cyclopentyloxy-phenyl)-piperazin-1-yl]-2-hydroxy-propyl}-3-cyclopropylamino-4-methyl-pyrrolidine-2,5-dione. Yield: 0.4 gm (70%)

### Step 5: Preparation of 1-{3-[4-(2-cyclopentyloxy-phenyl)-piperazin-1-yl]-2-hydroxy-propyl}-3-cyclopropylamino-4-methyl-pyrrolidine-2,5-dione hydrochloride salt

The hydrochloride salt of 1-{3-[4-(2-cyclopentyloxy-phenyl)-piperazin-1-yl]-2-hydroxy-propyl}-3-cyclopropylamino-4-methyl-pyrrolidine-2,5-dione was prepared following the previously disclosed procedure of Example 1, Step 5. Yield: 0.35 gm (85%)
IR (KBr): 1699.6 cm⁻¹; Mass (m/z): 471 (M⁺+1)

The following compounds were prepared by following above procedure:
Compound No. 40: 1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione hydrochloride salt
IR (KBr): 1682.7 cm⁻¹; Mass (m/z): 489 (M⁺+1)
Compound No. 42: 1-{3-[4-(2-Cyclopentyloxy-5-fluoro-phenyl)-piperazin-1-yl]-2-hydroxy-propyl}-3-methyl-4-methylamino-pyrrolidine-2,5-dione hydrochloride salt
IR (KBr): 1693 cm⁻¹; Mass (m/z): 489 (M⁺+1)
Compound No. 46: 1-{3-[4-(2-Cyclopentyloxy-5-fluoro-phenyl)-piperazin-1-yl]-2-hydroxy-propyl}-3-methyl-4-methylamino-pyrrolidine-2,5-dione hydrochloride salt
IR (KBr): 1704.6 cm⁻¹; Mass (m/z): 463 (M⁺+1)
Compound No. 110: 3-Cyclopropylaminomethyl-1-{2-hydroxy-3-[4-(2-methoxy-phenyl)-piperazin-1-yl]-propyl}-4-methyl-pyrrolidine-2,5-dione hydrochloride salt
IR (KBr): 1657 cm⁻¹; Mass (m/z): 417 (M⁺+1)
Compound No. 112: 3-Cyclopropylaminomethyl-1-{2-hydroxy-3-[4-(2-methoxy-phenyl)-piperazin-1-yl]-propyl}-4-methyl-pyrrolidine-2,5-dione hydrochloride salt
IR (KBr): 1625 cm⁻¹; Mass (m/z): 417 (M⁺+1)
Compound No. 114: 3-Cyclobutylaminomethyl-1-{2-hydroxy-3-[4-(2-methoxy-phenyl)-piperazin-1-yl]-propyl}-pyrrolidine-2,5-dione hydrochloride salt
IR (KBr): 1704 cm⁻¹; Mass (m/z): 431 (M⁺+1)

### Example 16

### Preparation of 1-(2-Hydroxy-3-[4-(2-methoxy-phenyl)-piperazin-1-yl]-propyl}-3-methyl-pyrrolidine-2,5-dione hydrochloride salt (Compound No. 116)

### Step 1: Preparation of 1-{2-Hydroxy-3-[4-(2-methoxy-phenyl)-piperazin-1-yl]-propyl}-3-methyl-pyrrolidine-2,5-dione

To a clear solution of 1-{2-Hydroxy-3-[4-(2-methoxy-phenyl)-piperazin-1-yl]-propyl}-3-methyl-pyrrole-2,5-dione (0.8 gm, 0.0022 mol) in methanol (15 mL) Pd/Carbon (0.4 gm) was added and the reaction mixture was hydrogenated at 40-45 psi for 1 hour. The reaction mixture was filtered through a celite pad and washed with methanol; the filtrate was concentrated to yield 1-{2-Hydroxy-3-[4-(2-methoxy-phenyl)-piperazin-1-yl]-propyl}-3-methyl-pyrrolidine-2,5-dione. Yield: 0.8 gm (99%)

### Step 2: Preparation of 1-{2-Hydroxy-3-[4-(2-methoxy-phenyl)-piperazin-1-yl]-propyl}-3-methyl-pyrrolidine-2,5-dione hydrochloride salt

The hydrochloride salt of 1-{2-Hydroxy-3-[4-(2-methoxy-phenyl)-piperazin-1-yl]-propyl}-3-methyl-pyrrolidine-2,5-dione was prepared following the previously disclosed procedure of Example 1, Step 5. Yield: 0.72 g (90%)
IR (KBr): 1693 cm⁻¹; Mass (m/z): 362 (M⁺+1)

The following compound was similarly prepared following the above procedure:
Compound No. 158: 2-{3-[4-(2-Cyclopentyloxy-phenyl)-piperazin-1-yl]-propyl)-4,7-dihydroxy-hexahydro-isoindole-1,3-dione hydrochloride salt
IR (KBr): 1703 cm⁻¹; Mass (m/z): 472 (M⁺+1)

### Example 17

### Preparation of 5-Fluoro-6-hydroxy-2-{2-hydroxy-3-[4-(2-isopropoxy-phenyl)-piperazin-1-yl]-propyl}-hexahydro-isoindole-1,3-dione hydrochloride salt (Compound No. 62)

### Step 1: Preparation of 2-{2-hydroxy-3[4-(2-isopropoxy-phenyl)-piperazin-1-yl]-propyl}-3a,4,7,7a-tetrahydro-isoindole-1,3-dione

A mixture of 1-amino-3-[4-(2-isopropoxyphenyl)piperazin-1-yl]propan-2-ol (0.7 gm, 0.0023 mol) and tetrahydrophthalic anhydride (0.36 gm, 0.0024 mol) in toluene (15 mL) was refluxed for about 1 hour. The reaction mixture was concentrated and the crude product was purified anhydrous column chromatography to yield 2-{2-hydroxy-3[4-(2-isopropoxy-phenyl)-piperazin-1-yl]-propyl}-3a,4,7,7a-tetrahydro-isoindole-1,3-dione. Yield:0.9 gm (90%)

### Step 2: Preparation of 5,6-dihydroxy-2-{2-hydroxy-3[4-(2-isopropoxy-phenyl)-piperazin-1-yl]-propyl}-hexahydro-isoindole-1,3-dione

To a solution of 2-{2-hydroxy-3[4-(2-isopropoxy-phenyl)-piperazin-1-yl]-propyl}-3a,4,7,7a-tetrahydro-isoindole-1,3-dione (1.0 gm, 0.0023 mol) in ethanol (20 mL) was added potassium permanganate solution (0.44 gm, 0.0028 mole) at 0-5° C. The reaction mixture was stirred for about 6-8 hours. After completion of the reaction, the reaction mixture was filtered through a celite pad; washed with ethanol; the combined filtrate was concentrated; and the crude product was purified by column purification to yield 5,6-dihydroxy-2-{2-hydroxy-3[4-(2-isopropoxy-phenyl)-piperazin-1-yl]-propyl}-hexahydro-isoindole-1,3-dione. Yield: 0.54 gm, 50%

### Step 3: Preparation of 5-Fluoro-6-hydroxy-2-{2-hydroxy-3-[4-(2-isopropoxy-phenyl)-piperazin-1-yl]-propyl}-hexahydro-isoindole-1,3-dione

To a solution of 5,6-dihydroxy-2-{2-hydroxy-3[4-(2-isopropoxy-phenyl)-piperazin-1-yl]-propyl}-hexahydro-isoindole-1,3-dione (1.0 gm, 0.0022 mol) in dichloromethane (10 mL) was added diethylamino sulfur trifluoride (0.422 gm, 0.0026 mol) at 0-5° C. and the reaction mixture stirred for 2-3 hours. The reaction mixture was quenched by adding water (20 mL); extracted with dichloromethane; the organic layer was concentrated; and the crude product was purified by column chromatography to yield 5-Fluoro-6-hydroxy-2-{2-hydroxy-3-[4-(2-isopropoxy-phenyl)-piperazin-1-yl]-propyl}-hexahydro-isoindole-1,3-dione. Yield: 0.25 gm (25%)

### Step 4: Preparation of 5-Fluoro-6-hydroxy-2-{2-hydroxy-3-[4-(2-isopropoxy-phenyl)-piperazin-1-yl]-propyl}-hexahydro-isoindole-1,3-dione hydrochloride salt

The hydrochloride salt of 2-{3-[4-(2-Cyclopentyloxy-phenyl)-piperazin-1-yl]-propyl)-4,7-dihydroxy-hexahydro-isoindole-1,3-dione was prepared following the previously disclosed procedure of Example 1, Step 5. Yield: 0.22 g (90%)
IR (KBr): 1699.2 cm⁻¹; Mass (m/z): 464 (M⁺+1)

### Pharmacological Testing

### Receptor Binding Assay

Receptor binding assays were performed using native α-1 adrenoceptors. The affinity of different compounds for α₁ₐ and α_{1b} adrenoceptor subtypes was evaluated by studying their ability to displace specific [³H]prazosin binding from the membranes of rat submaxillary and liver respectively (Michel et al., Br J Pharmacol, 98:883-889 (1989)). The binding assays were performed according to U'Prichard et al, Eur J Pharmacol, 50:87-89 (1978) with minor modifications.

Submaxillary glands were isolated immediately after sacrifice. The liver was perfused with buffer (Tris hydrochloric acid 50 mM, sodium chloride 100 mM, 10 mM ethylene diamine tetra acetic acid pH 7.4). The tissues were homogenized in 10 volumes of buffer (Tris HCl 50 mM, NaCl 100 mM, EDTA 10 mM, pH 7.4). The homogenate was filtered through two layers of wet gauze and the filtrate was centrifuged at 500 g for 10 min. The supernatant was subsequently centrifuged at 40,000 g for 45 min. The pellet thus obtained was resuspended in the same volume of assay buffer (Tris HCl 50 mM, EDTA 5 mM, pH 7.4) and were stored at −70° C. until the time of assay.

The membrane homogenates (150-250 μg protein) were incubated in 250 μL of assay buffer (Tris HCl 50 mM, EDTA 5 mM, pH 7.4) at 24-25° C. for 1 hour. Non-specific binding was determined in the presence of 300 nM prazosin. The incubation was terminated by vaccum filtration over GF/B fiber filters. The filters were then washed with ice cold 50 mM Tris HCl buffer (pH 7.4). The filtermats were dried and bounded radioactivity retained on filters was counted. The IC₅₀ and Kd were estimated by using the non-linear curve-fitting program using G pad prism software. The value of inhibition constant Ki was calculated from competitive binding studies by using Cheng and Prusoff equation (Cheng and Prusoff, Biochem Pharmacol, 22:3099-3108 (1973)), Ki=IC₅₀/(1+L/Kd) where L is the concentration of [³H] prazosin used in the particular experiment.

The Ki values for compounds disclosed herein range as follows:

a) α₁ₐ Ki (nM) for compounds disclosed herein were between about 0.1 nM to about 590 nM, as well as between about 0.5 nM to about 200 nM, even between about 1 nM to about 50 nM.

b) α_{1b} Ki (nM) for compounds disclosed herein were between about 9 nM to greater than about 10,000 nM, as well as between about 30 nM to about 700 nM, even between about 100 nM to about 500 nM.

In vitro Functional Studies (In vitro α₁ₐ Adrenoceptor Selectivity)

In order to study selectivity of action of the present compounds towards different α₁ₐ adrenoreceptor subtypes, the ability of these compounds to antagonize α₁ₐ adrenoreceptor agonist induced contractile response of aorta (α_{1d}), prostate (α₁ₐ) and spleen (α_{1b}) was studied. Aorta, prostate and spleen tissue were isolated from thiopentone-anaesthetized (≈300 mg/Kg) male wistar rats. Isolated tissues were mounted in organ bath containing Krebs Henseleit buffer of the following composition (mM): sodium chloride (NaCl) 118; potassium chloride (KCl) 4.7; calcium chloride (CaCl₂) 2.5; magnesium sulfate heptahydrate (MgSO₄. 7H₂O) 1.2; sodium bicarbonate (NaHCO₃) 25; potassium dihydrogen phosphate (KH₂PO₄) 1.2; glucose 11.1. The buffer was maintained at 37° C. and aerated with a mixture of 95% oxygen (O₂) and 5% carbon dioxide (CO₂). A resting tension of 2 g (aorta and spleen) or 1 g (prostate) was applied to tissues. Contractile response was monitored using a force displacement transducer and recorded on chart recorders. Tissues were allowed to equilibrate for 1½ hour. At the end of equilibration period, concentration response curves to norepinephrine (aorta) and phenylepinephrine (spleen and prostate) were obtained in the absence and presence of the tested compound (at concentration of 0.1, 1 and 10 μM).

In vitro functional assays of the compounds disclosed herein resulted in the following pKB values:

a) α₁ₐ (pKB) values were between about 8.1 to about 9.7, between about 8.5 to about 9.4, even between about 8.7 to about 9.1;

b) α_{1b} (pKB) values were between about 6.7 to about 8.2, between about 7.4 to about 8.0, even between about 7.7 to about 7.9.

### Human Recombinant Assay

Receptor Binding Assay: Receptor binding assays were performed using recombinant cells, expressing human alpha-1a and alpha-1b adrenoceptors. The affinity of different compounds for α₁ₐ and α_{1b} adrenoceptor subtypes was evaluated by studying their ability to displace specific [³H] prazosin binding from the membranes of recombinant clones expressing alpha-1a and alpha-1b adrenoceptors. The binding assays were performed according to U'Prichard et al., Eur J Pharmacol, 50:87-89 (1978) with minor modifications.

Human embryonic kidney (HEK) cells which had been stably transfected with human alpha-1a and alpha-1b adrenoceptors were cultured in an atmosphere of 5% CO₂at 37° C. in DMEM medium supplemented with 10% heat inactivated fetal calf serum, 1 mM glutamine, 100 U/mL penicillin and 0.1 mg/mL streptomycin. Selection pressure was maintained by regular addition of puromycin (3 μg/mL) to the culture medium.

The cells were homogenized in 5-10 volumes of buffer (Tris HCl 5 mM, EDTA 5 mM, pH 7.4) using a polytron homogenizer. The homogenate was centrifuged at 40,000 g for 20 min at 4° C. The pellet thus obtained was resuspended in assay buffer (Tris HCl 5 mM, EDTA 5 mM, pH 7.4) and were stored at −70° C. until the time of assay.

Competition radioligand binding to the cloned subtypes of α₁-adrenoceptors was performed using [³H] prazosin as the radioligand¹. The membrane homogenates (5-10 [2g protein) were incubated in 250 μL of assay buffer (Tris HCl 50 mM, EDTA 5 mM, pH 7.4) at 24-25° C. for 1 hour. Non-specific binding was determined in the presence of 10 μM terazosin. The incubation was terminated by vacuum filtration over GF/B fiber filters. The filters were then washed with ice-cold 50 mM Tris HCl buffer (pH 7.4). The filter mats were dried and bounded radioactivity retained on filters was counted. The IC₅₀ and Kd were estimated by using the non-linear curve-fitting program using Graph pad prism software. The value of inhibition constant Ki was calculated from competitive binding studies by using Cheng and Prusoff equation (Cheng and Prusoff, Biochem Pharmacol, 22:3099-3108 (1973)), Ki=IC₅₀ /(1+L/Kd) where L is the concentration of [³H] prazosin used in the particular experiment.

Reference: Michel, M. C, Grübbel, B., Taguchi, K. et al: Drugs for treatment of benign prostatic hyperplasia: affinity comparison at cloned cm -adrenoceptor subtypes and in human prostate. J. Auton Pharmacol, 16:21 (1996).

The results of the human recombinant assays of the compounds disclosed herein are as follows:

a) The compounds disclosed herein exhibited α₁ₐ Ki (nM) values of between about 0.2 nM to about 415 nM, between about 1 nM to about 150 nM, and even between about 3 nM to about 50 nM;

The compounds disclosed herein exhibited α_{1b} Ki (nM) values of between about 0.5 nM to about 1715 nM, between about 20 nM to about 800 nM, and even between about 50 nM to about 550 nM.

1. A compound having the structure of Formula I, 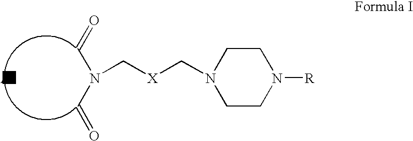
pharmaceutically acceptable salts, pharmaceutically acceptable solvates, enantiomers, diastereomers, n-oxides, prodrugs, polymorphs and metabolites thereof, wherein:
A is 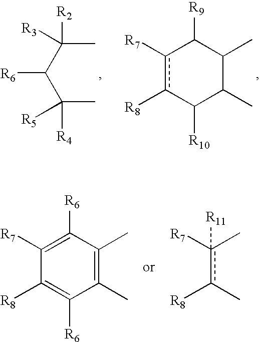
wherein, R₂, R₃, R₄ and R₅ are independently hydrogen, alkyl or phenyl, R₆, is hydrogen, alkyl, phenyl, hydroxy or alkoxy, R₇ and R₈ are each independently hydrogen, alkyl, alkynyl, cycloalkyl, halogen, hydroxy, aryl, acetoxy, heterocycle, 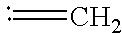
(wherein [symbol] is the point of attachment) or R₁₂-Q-(CH₂)ₘ— (wherein m is an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or 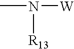
(wherein, W is no atom, carbonyl, carboxylate or amide, R₁₃ is hydrogen, alkyl, cycloalkyl, aryl or heterocycle), R₇ and R₈ together is cycloalkyl, cycloalkenyl, bicyclic alkyl, bicyclic alkenyl, aryl, heterocycle or 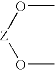
(wherein Z is CO or SO), R₉ and R₁₀ are each independently hydrogen, hydroxy, alkoxy, acetyl, or acetyloxy, R₁₁ is hydrogen, alkyl, alkenyl, alkynyl, cycloalkyl, aryl, or heterocycle, no atom:
X is CO, CS or CHY (wherein Y is hydrogen, hydroxy, halogen, alkoxy or haloalkoxy); and
R is alkyl, alkenyl, alkynyl, cycloalkyl, aryl or heterocycle;
with the provisos that
(a) when A is 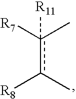
X is —CH₂— and R₁₁ is hydrogen then R₇ is hydrogen or alkyl with the further proviso that when R₇ is alkyl and R₈ is R₁₂ NH—, then R₁₂ is substituted alkyl wherein the substituents are selected from aryl or heterocyclyl,
(b) when A is 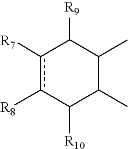
and X is —CH₂—, then none of R₇, R₈, R₉ or R₁₀ are hydrogen or halogen.
(c) when A is 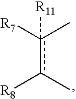
X is —CH₂—, and R₁₁ is no atom, then R₇ can be ═CH₂.

2. The compound of claim 1, wherein:
A is 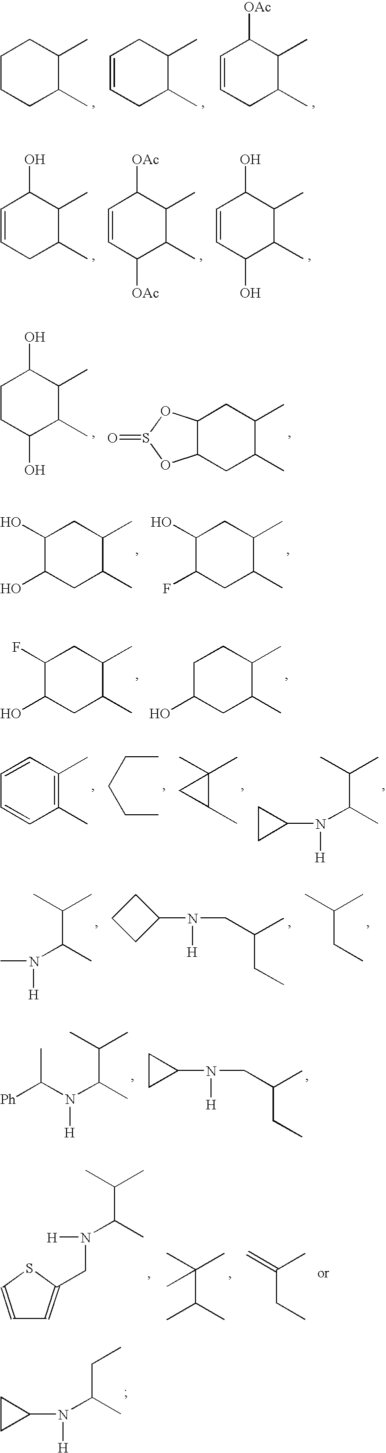
X is CHOH, CO, CH₂ or CHF;
R is: 2-methoxy phenyl, 3-fluoro-2-methoxy phenyl, 5-fluoro-2-methoxy phenyl, 4-fluoro-2-methoxyphenyl, 2-methoxy-5-methyl phenyl, 2-n-propoxyphenyl, 5-fluoro-2-n-propoxyphenyl, 2-ethoxy phenyl, 2-isopropoxy phenyl, 4-fluoro-2-isopropoxyphenyl, 4-nitro-2-isopropoxyphenyl, 3-fluoro-2-isopropoxy phenyl, 5-fluoro-2-isopropoxy phenyl, 2-cyclopentoxy-5-fluoro phenyl, 2-cyclopentoxy phenyl, O-tolyl, 2-trifluoroethoxy phenyl, 5-fluoro-2-trifluoromethoxy phenyl or 2-(2,2,3,3-tetrafluoropropoxy)phenyl.

3. A compound, which is:
2-{2-Hydroxy-3-[4-(2-isopropoxy-phenyl)-piperazin-1-yl]-propyl}-hexahydro-isoindole-1,3-dione,
2-{2-Hydroxy-3-[4-(2-isopropoxy-phenyl)-piperazin-1-yl]-propyl}-hexahydro-isoindole-1,3-dione hydrochloride salt,
2-{3-[4-(2-Isopropoxy-phenyl)-piperazin-1-yl]-2-oxo-propyl}-hexahydroisoindole-1,3-dione,
2-{3-[4-(2-Isopropoxy-phenyl)-piperazin-1-yl]-2-oxo-propyl}-hexahydroisoindole-1,3-dione hydrochloride salt,
2-{3-[4-(2-Isopropoxy-phenyl)-piperazin-1-yl]-2-oxo-propyl}-3a,4,7,7a-tetrahydro-isoindole-1,3-dione,
2-{3-[4-(2-Isopropoxy-phenyl)-piperazin-1-yl]-2-oxo-propyl}-3a.4,7,7a-tetrahydro-isoindole-1,3-dione hydrochloride salt,
2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione,
2-{(S)-2-Hydroxy-3-{4-[2-(2,2,2-trifluoro-ethoxy)-phenyl]-piperazin-1-yl}-propyl)-3a,4,7,7a-tetrahydro-isoindole-1,3-dione hydrochloride salt,
2-(2-Hydroxy-3-{4-[2-(2,2,3,3-tetrafluoro-propoxy)-phenyl]-piperazin-1-yl}-propyl)-3a,4,7,7a-tetrahydro-isoindole-l, 3-dione,
2-(2-Hydroxy-3-{4-[2-(2,2,3,3-tetrafluoro-propoxy)-phenyl]-piperazin-1-yl}-propyl)-3a,4,7,7a-tetrahydro-isoindole-1,3-dione hydrochloride salt,
2-{2-Hydroxy-3-[4-(2-isopropoxy-4-nitro-phenyl)-piperazin-1-yl]-propyl}-3a,4,7,7a-tetrahydro-isoindole-1,3-dione,
2-{2-Hydroxy-3-[4-(2-isopropoxy-4-nitro-phenyl)-piperazin-1-yl]-propyl}-3a,4,7,7a-tetrahydro-isoindole-1,3-dione hydrochloride salt,
2-{2-Fluoro-3-[4-(2-isopropoxy-phenyl)-piperazin-1-yl]-propyl}-3a,4,7,7a-tetrahydro-isoindole-1,3-dione,
2-{2-Fluoro-3-[4-(2-isopropoxy-phenyl)-piperazin-1-yl]-propyl}-3a,4,7,7a-tetrahydro-isoindole-1,3-dione hydrochloride salt,
Acetic acid 2-{3-[4-(2-methoxy-phenyl)-piperazin-1-yl]-propyl}-1,3-dioxo-2,3,3a,4,7,7a-hexahydro-1H-inden-4-yl ester,
Acetic acid 2-{3-[4-(2-methoxy-phenyl)-piperazin-1-yl]-propyl}-1,3-dioxo-2,3,3a,4,7,7a-hexahydro-1H-inden-4-yl ester hydrochloride salt,
4-Hydroxy-2-{3-[4-(2-methoxy-phenyl)-piperazin-1-yl]-propyl}-3a,4,7,7a-tetrahydro-isoindole-1,3-dione,
4-Hydroxy-2-{3-[4-(2-methoxy-phenyl)-piperazin-1-yl]-propyl}-3a,4,7,7a-tetrahydro-isoindole-1,3-dione hydrochloride salt,
2-{3-[4-(2-Methoxy-phenyl)-piperazin-1-yl}-2-oxo-propyl}-3a,4,7,7a-tetrahydro-isoindole-1,3-dione,
2-{3-[4-(2-Methoxy-phenyl)-piperazin-1-yl}-2-oxo-propyl}-3a,4,7,7a-tetrahydro-isoindole-1,3-dione hydrochloride salt,
2-{3-[4-(2-Cyclopentyloxy-phenyl)-piperazin-1-yl]-2-oxo-propyl}-3a,4,7,7a-tetrahydro-isoindole-1,3-dione,
2-{2-Oxo-3-[4-(2-propoxy-phenyl)-piperazin-1-yl]-propyl}-3a,4,7,7a-tetrahydro-isoindole-1,3-dione,
2-{2-Oxo-3-[4-(2-propoxy-phenyl)-piperazin-1-yl]-propyl}-3a,4,7,7a-tetrahydro-isoindole-1,3-dione hydrochloride salt,
2-{3-[4-(4-Fluoro-2-isopropoxy-phenyl)-piperazin-1-yl]-2-oxo-propyl}-3a,4,7,7a-tetrahydro-isoindole-1,3-dione,
2-{3-[4-(4-Fluoro-2-isopropoxy-phenyl)-piperazin-1-yl]-2-oxo-propyl}-3a,4,7,7a-tetrahydro-isoindole-1,3-dione hydrochloride salt,
2-{3-[4-(2-Isopropoxy-phenyl)-piperazin-1-yl]-2-oxo-propyl}-isoindole-1,3-dione,
2-{3-[4-(2-Isopropoxy-phenyl)-piperazin-1-yl]-2-oxo-propyl}-isoindole-1,3-dione hydrochloride salt,
2-(2-Oxo-3-{4-[2-(2,2,2-trifluoro-ethoxy)-phenyl]-piperazin-1-yl}-propyl)-3a,4,7,7a-tetrahydro-isoindole-1,3-dione,
2-(2-Oxo-3-{4-[2-(2,2,2-trifluoro-ethoxy)-phenyl]-piperazin-1-yl}-propyl)-3a,4,7,7a-tetrahydro-isoindole-1,3-dione hydrochloride salt,
6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione,
6-{3-[4-(2-Isopropoxy-phenyl)-piperazin-1-yl]-propyl}-2-oxo-hexahydro-1,3-dioxa-2-lambda*4*-thia-6-aza-s-indacene-5,7-dione hydrochloride salt,
1-[2-Oxo-3-(4-phenyl-piperazin-1-yl)-propyl]-pyrrolidine-2,5-dione,
1-{3-[4-{4-Fluoro-phenyl}-piperazin-1-yl]-2-oxo-propyl}-3-phenyl-piperidine-2,6-dione,
3,4-Dimethyl-1-{2-oxo-3-[4-(2-trifluoromethyl-phenyl)-piperazin-1-yl]-propyl}-pyrrole-2,5-dione,
1-{2-Fluoro-3-[4-(4-fluorophenyl)piperazin-1-yl]-propyl}-piperidine-2,6-dione,
1-(2-Fluoro-3-{4-[2-(2,2,2-trifluoro-ethoxy)-phenyl]-piperazin-1-yl}propyl)-3,4-dimethylpyrrole-2,5-dione,
1-{3-[4-(2-Cyclopentyloxy-phenyl)-piperazin-1-yl]-2-hydroxy-propyl}-3-cyclopropylamino-4-methyl-pyrrolidine-2,5-dione,
1-{3-[4-(2-Cyclopentyloxy-phenyl)-piperazin-1-yl]-2-hydroxy-propyl}-3-cyclopropylamino-4-methyl-pyrrolidine-2,5-dione hydrochloride salt,
1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione,
1-{3-[4-(2-Cyclopentyloxy-5-fluoro-phenyl)-piperazin-1-yl]-2-hydroxy-propyl}-3-cyclopropylamino-4-methyl-pyrrolidine-2,5-dione hydrochloride salt,
1-{3-[4-(2-Cyclopentyloxy-5-fluoro-phenyl)-piperazin-1-yl]-2-hydroxy-propyl}-3-cyclopropylaminomethyl-pyrrolidine-2,5-dione,
1-{3-[4-(2-Cyclopentyloxy-5-fluoro-phenyl)-piperazin-1-yl]-2-hydroxy-propyl}-3-cyclopropylaminomethyl-pyrrolidine-2,5-dione hydrochloride salt,
2-{3-[4-(5-Fluoro-2-isopropoxy-phenyl)-piperazin-1-yl]-propyl}-5,6-dihydroxy-hexahydro-isoindole-1,3-dione,
2-{3-[4-(5-Fluoro-2-isopropoxy-phenyl)-piperazin-1-yl]-propyl}-5,6-dihydroxy-hexahydro-isoindole-1,3-dione hydrochloride salt,
1-{3-[4-{2-Cyclopentyloxy-5-fluoro-phenyl)-piperazin-1-yl]-2-hydroxy-propyl}-3-methyl-4-methylamino-pyrrolidine-2,5-dione,
1-{3-[4-(2-Cyclopentyloxy-5-fluoro-phenyl)-piperazin-1-yl]-2-hydroxy-propyl}-3-methyl-4-methylamino-pyrrolidine-2,5-dione hydrochloride salt,
1-{3-[4-(2-Methoxy-5-methyl-phenyl)-piperazin-1-yl]-propyl}-piperidine-2,6-dione,
1-{3-[4-(2-Methoxy-5-methyl-phenyl)-piperazin-1-yl]-propyl}-piperidine-2,6-dione hydrochloride salt,
5,6-Dihydroxy-2-{3-[4-(2-methoxy-5-methyl-phenyl)-piperazin-1-yl]-propyl}-hexahydro-isoindole-1,3-dione,
5,6-Dihydroxy-2-{3-[4-(2-methoxy-5-methyl-phenyl)-piperazin-1-yl]-propyl}-hexahydro-isoindole-1,3-dione hydrochloride salt,
1-(3-{4-Fluoro-2-(2,2,2-trifluoro-ethoxy)-phenyl]-piperazin-1-yl}-propyl)-piperidine-2,6-dione,
1-(3-{4-[5-Fluoro-2-(2,2,2-trifluoro-ethoxy)-phenyl]-piperazin-1-yl}-propyl)-piperidine-2,6-dione hydrochloride salt,
2-{3-[4-(2-Cyclopentyloxy-phenyl)-piperazin-1-yl]-propyl}-5,6-dihydroxy-hexahydro-isoindole-1,3-dione,
2-(3-[4-(2-Cyclopentyloxy-phenyl)-piperazin-1-yl]-propyl}-5,6-dihydroxy-hexahydro-isoindole-1,3-dione hydrochloride salt,
3-{3-[4-(3-Fluoro-2-methoxy-phenyl)-piperazin-1-yl]-propyl)-1-methyl-3-aza-bicyclo[3,1.0]hexane-2,4-dione,
3-{3-[4-(3-Fluoro-2-methoxy-phenyl)-piperazin-1-y 1 ]-propyl)-1-methyl-3-aza-bicyclo[3.1.0]hexane-2,4-dione hydrochloride salt,
3-{3-[4-(5-Fluoro-2-methoxy-phenyl)-piperazin-1-yl]-propyl}-1-methyl-3-aza-bicyclo[3.1.0]hexane-2,4-dione,
3-{3-[4-(5-Fluoro-2-methoxy-phenyl)-piperazin-1-yl]-propyl}-1-methyl-3-aza-bicyclo[3.1.0]hexane-2,4-dione hydrochloride salt,
3-{3-[4-(2-Cyclopentyloxy-phenyl)-piperazin-1-yl3-propyl}-1-methyl-3-aza-bicyclo[3.1.0]hexane-2,4-dione,
3-{3-[4-(2-Cyclopentyloxy-phenyl)-piperazin-1-yl]-propyl}-1-methyl-3-aza-bicyclo[3.1.0]hexane-2,4-dione hydrochloride salt,
5-Fluoro-6-hydroxy-2-{2-hydroxy-3-[4-(2-isopropoxy-phenyl)-piperazin-1-yl]-propyl}-hexahydro-isoindole-1,3-dione,
5-Fluoro-6-hydroxy-2-{2-hydroxy-3-[4-(2-isopropoxy-phenyl)-piperazin-1-yl]-propyl}-hexahydro-isoindole-1,3-dione hydrochloride salt,
3-{3-[4-(5-Fluoro-2-isopropoxy-phenyl)-piperazin-1-yl]-propyl}-1-methyl-3-aza-bicyclo[3,1.0]hexane-2,4-dione,
3-{3-[4-(5-Fluoro-2-isopropoxy-phenyl)-piperazin-1-yl]-propyl}-1-methyl-3-aza-bicyclo[3.1.0]hexane-2,4-dione hydrochloride salt,
5-Fluoro-6-hydroxy-2-{3-[4-(2-isopropoxy-phenyl)-piperazin-1-yl]-propyl}-hexahydro-isoindole-1,3-dione,
5-Fluoro-6-hydroxy-2-{3-[4-(2-isopropoxy-phenyl)-piperazin-1-yl]-propyl}-hexahydro-isoindole-1,3-dione hydrochloride salt,
2-{3-[4-(2-Cyclopentyloxy-phenyl)-piperazin-1-yl]-2-hydroxy-propyl}-hexahydro-isoindole-1,3-dione,
2-{3-[4-(2-Cyclopentyloxy-phenyl)-piperazin-1-yl]-2-hydroxy-propyl}-hexahydro-isoindole-1,3-dione hydrochloride salt,
5-Hydroxy-2-{3-[4-(2-methoxy-phenyl)-piperazin-1-yl]-propyl}-hexahydro-isoindole-1,3-dione,
5-Hydroxy-2-{3-[4-(2-methoxy-phenyl)-piperazin-1-yl]-propyl}-hexahydro-isoindole-1,3-dione hydrochloride salt,
2-{3-[4-(2-Cyclopentyloxy-5-fluoro-phenyl)-piperazin-1-yl]-propyl}-5-hydroxy-hexahydro-isoindole-1,3-dione,
2-{3-[4-(2-Cyclopentyloxy-5-fluoro-phenyl)-piperazin-1-yl]-propyl}-5-hydroxy-hexahydro-isoindole-1,3-dione hydrochloride salt,
2-{3-[4-(2-Cyclopentyloxy-phenyl)-piperazin-1-yl]-propyl}-5-hydroxy-hexahydro-isoindole-1,3-dione,
2-{3-[4-(2-Cyclopentyloxy-phenyl)-piperazin-1-yl]-propyl}-5-hydroxy-hexahydro-isoindole-1,3-dione hydrochloride salt
2-{3-[4-(2-Cyclopentyloxy-phenyl)-piperazin-1-yl]-2-oxo-propyl}-5,6-dihydroxy-hexahydro-isoindole-1,3-dione,
2-{3-[4-(2-Cyclopentyloxy-phenyl)-piperazin-1-yl]-2-oxo-propyl}-5,6-dihydroxy-hexahydro-isoindole-1,3-dione hydrochloride salt,
2-{3-[4-(2-Cyclopentyloxy-5-fluoro-phenyl)-piperazin-1-yl]-2-oxo-propyl}-3a,4,7,7a-tetrahydro-isoindole-1,3-dione,
2-{3-[4-(2-Cyclopentyloxy-5-fluoro-phenyl)-piperazin-1-yl]-2-oxo-propyl}-3a,4,7,7a-tetrahydro-isoindole-1,3-dione hydrochloride salt,
2-{3-[4-(2-Cyclopentyloxy-5-fluoro-phenyl)-piperazin-1-yl]-2-hydroxy-propyl}-hexahydro-isoindole-1,3-dione,
2-{3-[4-(2-Cyclopentyloxy-5-fluoro-phenyl)-piperazin-1-yl]-2-hydroxy-propyl}-hexahydro-isoindole-1,3-dione hydrochloride salt,
5-Fluoro-2-{3-[4-(5-fluoro-2-methoxy-phenyl)-piperazin-1-yl]-propyl}-6-hydroxy-hexahydro-isoindole-1,3-dione,
5-Fluoro-2-{3-[4-(5-fluoro-2-methoxy-phenyl)-piperazin-1-yl]-propyl}-6-hydroxy-hexahydro-isoindole-1,3-dione hydrochloride salt,
2-{3-[4-(5-Fluoro-2-isopropoxy-phenyl)-piperazin-1-yl]-propyl}-5-hydroxy-hexahydro-isoindole-1,3-dione,
2-{3-[4-(5-Fluoro-2-isopropoxy-phenyl)-piperazin-1-yl]-propyl}-5-hydroxy-hexahydro-isoindole-1,3-dione hydrochloride salt,
5-Fluoro-2-{3-[4-(5-fluoro-2-isopropoxy-phenyl)-piperazin-1-yl]-propyl}-6-hydroxy-hexahydro-isoindole-1,3-dione,
5-Fluoro-2-{3-[4-(5-fluoro-2-isopropoxy-phenyl)-piperazin-1-yl]-propyl}-6-hydroxy-hexahydro-isoindole-1,3-dione hydrochloride salt,
1-{3-[4-(5-Fluoro-2-isopropoxy-phenyl)-piperazin-1-yl]-2-hydroxy-propyl}-piperidine-2,6-dione,
1-{3-[4-(5-Fluoro-2-isopropoxy-phenyl)-piperazin-1-yl]-2-hydroxy-propyl}-piperidine-2,6-dione hydrochloride salt,
1-{3-[4-(2-Cyclopentyloxy-5-fluoro-phenyl)-piperazin-1-yl]-2-hydroxy-propyl}-piperidine-2,6-dione,
1-{3-[4-(2-Cyclopentyloxy-5-fluoro-phenyl)-piperazin-1-yl]-2-hydroxy-propyl}-piperidine-2,6-dione hydrochloride salt,
Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester,
Acetic acid 7-acetoxy-2-{3-[4-(5-fluoro-2-isopropoxy-phenyl)-piperazin-1-yl]-propyl}-1,3-dioxo-2,3,3a,4,7,7a-hexahydro-1H-isoindol-4-yl ester hydrochloride salt,
Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester,
Acetic acid 7-acetoxy-2-{3-[4-(2-cyclopentyloxy-5-fluoro-phenyl)-piperazin-1-yl]-propyl}-1,3-dioxo-2,3,3a,4,7,7a-hexahydro-1H-isoindol-4-ylester hydrochloride salt,
2-{3-[4-(5-Fluoro-2-isopropoxy-phenyl)-piperazin-1-yl]-propyl}-4,7-dihydroxy-3a,4,7,7a-tetrahydro-isoindole-1,3-dione,
2-{3-[4-(5-Fluoro-2-isopropoxy-phenyl)-piperazin-1-yl]-propyl}-4,7-dihydroxy-3a,4,7,7a-tetrahydro-isoindole-1,3-dione hydrochloride salt,
1-{3-[4-(5-Fluoro-2-propoxy-phenyl)-piperazin-1-yl]-propyl}-piperidine-2,6-dione,
1-{3-[4-(5-Fluoro-2-propoxy-phenyl)-piperazin-1-yl]-propyl}-piperidine-2,6-dione hydrochloride salt,
1-{3-[4-(5-Fluoro-2-propoxy-phenyl)-piperazin-1-yl]-2-hydroxy-propyl)-piperidine-2,6-dione,
1-{3-[4-(5-Fluoro-2-propoxy-phenyl)-piperazin-1-yl]-2-hydroxy-propyl)-piperidine-2,6-dione hydrochloride salt,
2-{3-[4-(2-Cyclopentyloxy-5-fluoro-phenyl)-piperazin-1-yl]-propyl}-5,6-dihydroxy-hexahydro-isoindole-1,3-dione,
2-{3-[4-(2-Cyclopentyloxy-5-fluoro-phenyl)-piperazin-1-yl]-propyl}-5,6-dihydroxy-hexahydro-isoindole-1,3-dione hydrochloride salt,
2-{3-[4-(5-Fluoro-2-propoxy-phenyl)-piperazin-1-yl3-propyl}-5,6-dihydroxy-hexahydro-isoindole-1,3-dione,
2-{3-[4-(5-Fluoro-2-propoxy-phenyl)-piperazin-1-yl]-propyl}-5,6-dihydroxy-hexahydro-isoindole-1,3-dione hydrochloride salt,
2-{3-[4-(2-Cyclopentyloxy-phenyl)-piperazin-1-yl]-propyl}-5-fluoro-6-hydroxy-hexahydro-isoindole-1,3-dione,
2-{3-[4-(2-Cyclopentyloxy-phenyl)-piperazin-1-yl]-propyl}-5-fluoro-6-hydroxy-hexahydro-isoindole-1,3-dione hydrochloride salt,
2-{3-[4-(2-Cyclopentyloxy-5-fluoro-phenyl)-piperazin-1-yl]-propyl}-5-fluoro-6-hydroxy-hexahydro-isoindole-1,3-dione,
2-{3-[4-(2-Cyclopentyloxy-5-fluoro-phenyl)-piperazin-1-yl]-propyl}-5-fluoro-6-hydroxy-hexahydro-isoindole-1,3-dione hydrochloride salt,
3-Cyclopropylaminomethyl-1-{2-hydroxy-3-[4-(2-methoxy-phenyl)-piperazin-1-yl]-propyl}-pyrrolidine-2,5-dione,
3-Cyclopropylaminomethyl-1-{2-hydroxy-3-[4-(2-methoxy-phenyl)-piperazin-1-yl]-propyl}-pyrrolidine-2,5-dione hydrochloride salt,
3-Cyclopropylaminomethyl-1-{2-hydroxy-3-[4-(2-methoxy-phenyl)-piperazin-1-yl]-propyl}-4-methyl-pyrrolidine-2,5-dione,
3-Cyclopropylaminomethyl-1-{2-hydroxy-3-[4-(2-methoxy-phenyl)-piperazin-1-yl]-propyl}-4-methyl-pyrrolidine-2,5-dione hydrochloride salt,
3-Cyclobutylaminomethyl-1-{2-hydroxy-3-[4-(2-methoxy-phenyl)-piperazin-1-yl]-propyl}-pyrrolidine-2,5-dione,
3-Cyclobutylaminomethyl-1-{2-hydroxy-3-[4-(2-methoxy-phenyl)-piperazin-1-yl]-propyl}-pyrrolidine-2,5-dione hydrochloride salt,
1-{2-Hydroxy-3-[4-(2-methoxy-phenyl)-piperazin-1-yl]-propyl}-3-methyl-pyrrolidine-2,5-dione,
1-{2-Hydroxy-3-[4-(2-methoxy-phenyl)-piperazin-1-yl]-propyl}-3-methyl-pyrrolidine-2,5-dione hydrochloride salt,
2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione,
2-{3-[4-(2-Cyclopentyloxy-5-fluoro-phenyl)-piperazin-1-yl]-propyl}-4,7-dihydroxy-3a,4,7,7a-tetrahydro-isoindole-1,3-dione hydrochloride salt,
2-{3-[4-(2-Cyclopentyloxy-5-fluoro-phenyl)-piperazin-1-yl]-2-oxo-propyl}-5,6-dihydroxy-hexahydro-isoindole-1,3-dione,
2-{3-[4-(2-Cyclopentyloxy-5-fluoro-phenyl)-piperazin-1-yl]-2-oxo-propyl}-5,6-dihydroxy-hexahydro-isoindole-1,3-dione hydrochloride salt,
2-{3-[4-(5-Fluoro-2-isopropoxy-phenyl)-piperazin-1-y{]-2-oxo-propyl}-5,6-dihydroxy-hexahydro-isoindole-1,3-dione,
2-{3-[4-(5-Fluoro-2-isopropoxy-phenyl)-piperazin-1-yl]-2-oxo-propyl}-5,6-dihydroxy-hexahydro-isoindole-1,3-dione hydrochloride salt,
2-{3-[4-(5-Fluoro-2-isopropoxy-phenyl)-piperazin-1-yl]-2-hydroxy-propyl}-5,6-dihydroxy-hexahydro-isoindole-1,3-dione,
2-{3-[4-(5-Fluoro-2-isopropoxy-phenyl)-piperazin-1-yl]-2-hydroxy-propyl}-5,6-dihydroxy-hexahydro-isoindole-1,3-dione hydrochloride salt,
2-{3-[4-(2-Cyclopentyloxy-5-fluoro-phenyl)-piperazin-1-yl]-2-hydroxy-propyl}-5,6-dihydroxy-hexahydro-isoindole-1,3-dione,
2-{3-[4-(2-Cyclopentyloxy-5-fluoro-phenyl)-piperazin-1-yl]-2-hydroxy-propyl}-5,6-dihydroxy-hexahydro-isoindole-1,3-dione hydrochloride salt
1-{3-[4-(2-Cyclopentyloxy-5-fluoro-phenyl)-piperazin-1-yl]-propyl}-piperidine-}-piperidine-2,6-dione,
1-{3-[4-(2-Cyclopentyloxy-5-fluoro-phenyl)-piperazin-1-yl]-propyl}-piperidine-2,6-dione hydrochloride salt
1-{3-[4-(3-Fluoro-2-isopropoxy-phenyl)-piperazin-1-yl]-propyl}-piperidine-2,6-dione,
1-{3-[4-(3-Fluoro-2-isopropoxy-phenyl)-piperazin-1-yl]-propyl}-piperidine-2,6-dione hydrochloride salt,
1-{3-[4-(3-Fluoro-2-methoxy-phenyl)-piperazin-1-yl]-propyl}-piperidine-2,6-dione,
1-{3-[4-(3-Fluoro-2-methoxy-phenyl)-piperazin-1-yl]-propyl}-piperidine-2,6-dione hydrochloride salt,
1-{3-[4-(3-Fluoro-2-isopropoxy-phenyl)-piperazin-1-yl]-propyl}-3-methylene-pyrrolidine-2,5-dione,
1-{3-[4-(3-Fluoro-2-isopropoxy-phenyl)-piperazin-1-yl]-propyl}-3-methylene-pyrrolidine-2,5-dione hydrochloride salt,
1-{3-[4-(5-Fluoro-2-isopropoxy-phenyl)-piperazin-1-yl]-propyl}-3-methylene-pyrrolidine-2,5-dione,
1-{3-[4-(5-Fluoro-2-isopropoxy-phenyl)-piperazin-1-yl]-propyl}-3-methylene-pyrrolidine-2,5-dione hydrochloride salt,
1-{3-[4-(5-Fluoro-2-(2,2,3,3-tetrafluoro-propoxy)-phenyl3-piperazin-1-yl}-propyl)-piperidine-2,6-dione,
1-{3-[4-(5-Fluoro-2-(2,2,3,3-tetrafluoro-propoxy)-phenyl]-piperazin-1-yl}-propyl)-piperidine-2,6-dione hydrochloride salt,
1-{3-[4-(2-Methoxy-phenyl)-piperazin-1-yl]-propyl)-3-methyl-4-(1-phenyl-ethylamino)-pyrrolidine-2,5-dione,
1-{3-[4-(2-Methoxy-phenyl)-piperazin-1-yl]-propyl)-3-methyl-4-(1-phenyl-ethylamino)-pyrrolidine-2,5-dione hydrochloride salt,
1-{3-[4-(5-Fluoro-2-trifluoromethoxy-phenyl)-piperazin-1-yl]-propyl)-piperidine-2,6-dione,
1-{3-[4-(5-Fluoro-2-trifluoromethoxy-phenyl)-piperazin-1-yl]-propyl)-piperidine-2,6-dione hydrochloride salt,
Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-y1 ester,
Acetic acid 7-acetoxy-2-{3-[4-(2-ethoxy-phenyl)-piperazin-1-yl]-propyl)-1,3-dioxo-2,3,3a,4,7,7a-hexahydro-1H-isoindol-4-yl ester hydrochloride salt,
2-{3-[4-(2-Ethoxy-phenyl)-piperazin-1-yl]-propyl)-4,7-dihydroxy-3a,4,7,7a-tetrahydro-isoindole-1,3-dione,
2-{3-[4-(2-Ethoxy-phenyl)-piperazin-1-yl]-propyl)-4,7-dihydroxy-3a,4,7,7a-tetrahydro-isoindole-1,3-dione hydrochloride salt,
3-Cyclopropylamino-1-{3-[4-(2-ethoxy-phenyl)-piperazin-1-yl]-propyl}-pyrrolidine-2,5-dione,
3-Cyclopropylamino-1-{3-[4-(2-ethoxy-phenyl)-piperazin-1-yl]-propyl}-pyrrolidine-2,5-dione hydrochloride salt,
Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester,
Acetic acid 7-acetoxy-2-{3-[4-(2-methoxy-phenyl)-piperazin-1-yl]-propyl)-1,3-dioxo-2,3,3a,4,7,7a-hexahydro-1H-isoindol-4-yl ester hydrochloride salt,
1-{3-[4-(2-Cyclopentyloxy-phenyl)-piperazin-1-yl]-propyl)-3-cyclopropylamino-pyrrolidine-2,5-dione,
1-{3-[4-(2-Cyclopentyloxy-phenyl)-piperazin-1-yl]-propyl)-3-cyclopropylamino-pyrrolidine-2,5-dione hydrochloride salt,
4,7-Dihydroxy-2-{3-[4-(2-methoxy-phenyl)-piperazin-1-yl]-propyl)-3a,4,7,7a-tetrahydro-isoindole-1,3-dione,
4,7-Dihydroxy-2-{3-[4-(2-methoxy-phenyl)-piperazin-1-yl]-propyl)-3a,4,7,7a-tetrahydro-isoindole-1,3-dione hydrochloride salt,
Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester,
Acetic acid 7-acetoxy-2-{3-[4-(2-cyclopentyloxy-phenyl)-piperazin-1-yl]-propyl)-1,3-dioxo-2,3,3a,4,7,7a-hexahydro-1H-isoindol-4-yl ester hydrochloride salt,
2-{3-[4-(2-Cyclopentyloxy-phenyl)-piperazin-1-yl]-propyl)-4,7-dihydroxy-hexahydro-isoindole-1,3-dione,
2-{3-[4-(2Cyclopentyloxy-phenyl)-piperazin-1-yl]-propyl)-4,7-dihydroxy-hexahydro-isoindole-1,3-dione hydrochloride salt,
2-{3-[4-(2-Cyclopentyloxy-phenyl)-piperazin-1-yl]-propyl}-4,7-dihydroxy-3as4,7,7a-tetrahydro-isoindole-1,3-dione,
2-{3-[4-(2-Cyclopentyloxy-phenyl)-piperazin-1-yl]-propyl}-4,7-dihydroxy-3a,4,7,7a-tetrahydro-isoindole-1,3-dione hydrochloride salt,
3-Methylene-1-[3-(4-O-tolyl-piperazin-1-yl)-propyl]-pyrrolidine-2,5-dione,
3-Methylene-1-[3-(4-O-tolyl-piperazin-1-yl)-propyl]-pyrrolidine-2,5-dione hydrochloride salt,
1-{3-[4-(2-Methoxy-phenyl)-piperazin-1-yl]-propyl)-3-methyl-4-[(thiophen-2-ylmethyl)-amino]-pyrrolidine-2,5-dione,
1-{3-[4-(2-Methoxy-phenyl)-piperazin-1-yl]-propyl)-3-methyl-4-[(thiophen-2-ylmethyl)-amino]-pyrrolidine-2,5-dione hydrochloride salt,
1-{3-[4-(2-Cyclopentyloxy-phenyl)-piperazin-1-yl]-propyl}-3-methylene-pyrrolidine-2,5-dione,
1-{3-[4-(2-Cyclopentyloxy-phenyl)-piperazin-1-yl]-propyl}-3-methylene-pyrrolidine-2,5-dione hydrochloride salt,
1-{3-[4-(5-Fluoro-2-methoxy-phenyl)-piperazin-1-yl]-propyl}-3,3,4-trimethyl-pyrrolidine-2,5-dione,
1-{3-[4-(5-Fluoro-2-methoxy-phenyl)-piperazin-1-yl]-propyl}-3,3,4-trimethyl-pyrrolidine-2,5-dione hydrochloride salt,
1-{3-[4-(2-Cyclopentyloxy-phenyl)-piperazin-1-yl]-propyl}-piperidine-2,6-dione,
1-{3-[4-(2-Cyclopentyloxy-phenyl)-piperazin-1-yl]-propyl}-piperidine-2,6-dione hydrochloride salt,
or their pharmaceutically acceptable salts, pharmaceutically acceptable solvates, enantiomers, diastereomers, N-oxides, prodrugs, polymorphs or metabolites.

4. A pharmaceutical composition comprising a therapeutically effective amount of a compound of claim 1 and optionally one or more pharmaceutically acceptable carriers, excipients or diluents.

5. A method for treating a disease or disorder mediated through α₁ₐ and/or α_{1d} adrenergic receptors, comprising administering to patient in need thereof a therapeutically effective amount of a compound of claim 1 and optionally one or more pharmaceutically acceptable carriers, excipients or diluents.

6. The method according to claim 5, wherein disease or disorder is benign prostatic hyperplasia.

7. The method according to claim 5, wherein compound causes minimal decrease or no decrease in blood pressure at dosages effective to alleviate benign prostatic hyperplasia.

8. A method for treating lower urinary tract symptoms associated with or without benign prostatic hyperplasia, comprising administering to a patient in need thereof a therapeutically effective amount of a compound of claim 1 and optionally one or more pharmaceutically acceptable carriers, excipients or diluents.

9. A method for preparing a compound of Formula VII, 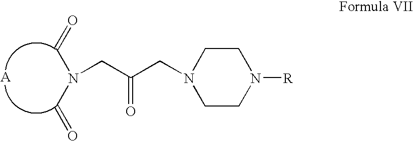
pharmaceutically acceptable salts, pharmaceutically acceptable solvates, enantiomers, diastereomers, n-oxides, prodrugs, polymorphs and metabolites thereof, wherein A is 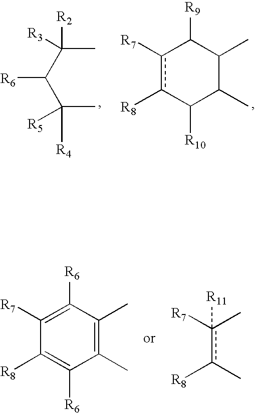
wherein, R₂, R₃, R₄ and R₅ are independently hydrogen, alkyl or phenyl, R₆ is hydrogen, alkyl, phenyl, hydroxy or alkoxy, R₇ and R₈ are each independently hydrogen, alkyl, alkynyl, cycloalkyl, halogen, hydroxy, aryl, acetoxy, heterocycle, 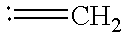
(wherein [symbol] is the point of attachment) or R₁₂-Q-(CH₂)ₘ— (wherein m is an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or 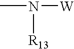
(wherein, W is no atom, carbonyl, carboxylate or amide, R₁₃ is hydrogen, alkyl, cycloalkyl, aryl or heterocycle), R₇ and R₈ together is cycloalkyl, cycloalkenyl, bicyclic alkyl, bicyclic alkenyl, aryl, heterocycle or 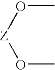
(wherein Z is CO or SO), R₉ and R₁₀ are each independently hydrogen, hydroxy, alkoxy, acetyl, or acetyloxy, R₁₁ is hydrogen, alkyl, alkenyl, alkynyl, cycloalkyl, and, or heterocycle, no atom;
X is CO, CS or CHY (wherein Y is hydrogen, hydroxy, halogen, alkoxy or haloalkoxy); and
R is alkyl, alkenyl, alkynyl, cycloalkyl, aryl or heterocycle;
with the provisos that
(i) when A is 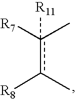
X is —CH₂— and R₁₁ is hydrogen then R₇ is hydrogen or alkyl with the further proviso that when R₇ is alkyl and R₈ is R₁₂ NH—, then R₁₂ is substituted alkyl wherein the substituents are selected from aryl or heterocyclyl,
(ii) when A is 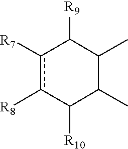
and X is —CH₂—, R₇, R₈, R₉ or R₁₀ are hydrogen or halogen,
which method comprises:
(a) reacting a compound of Formula II 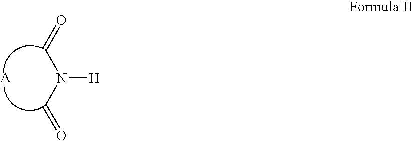
with 2-chloromethyl-oxirane 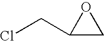
to form a compound of Formula III, 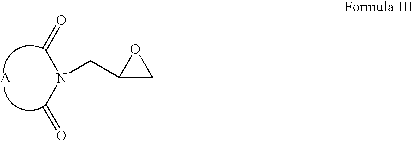
(b) reacting a compound of Formula III with hydrochloric acid to form a compound of Formula IV, 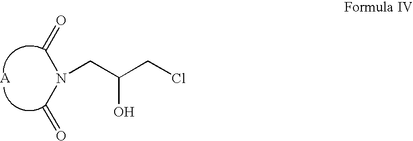
(c) oxidizing a compound of Formula IV to fonts a compound of Formula V, 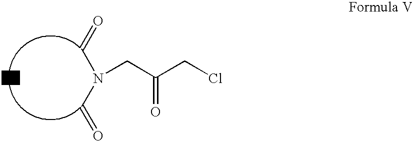
(d) treating a compound of Formula V with a compound of Formula VI 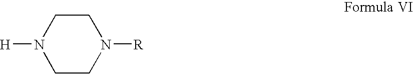
to form a compound of Formula VII.

10. A method for preparing a compound of Formulae VII or IX. 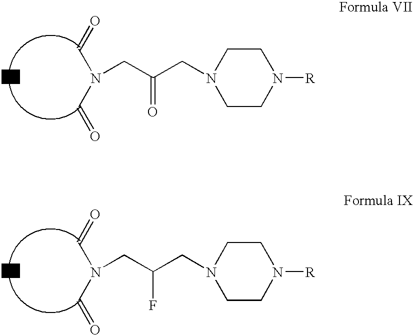
pharmaceutically acceptable salts, pharmaceutically acceptable solvates, enantiomers, diastereomers, n-oxides, prodrugs, polymorphs or metabolites thereof,
wherein A is 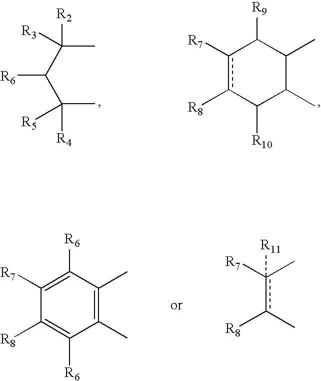
wherein, R₂, R₃, R₄ and R₅ are independently hydrogen, alkyl or phenyl, R₆, is hydrogen, alkyl, phenyl, hydroxy or alkoxy, R₇ and R₈ are each independently hydrogen, alkyl, alkynyl, cycloalkyl, halogen, hydroxy, aryl, acetoxy, heterocycle, 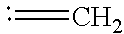
(wherein [symbol] is the point of attachment) or R₁₂-Q-(CH₂)ₘ— (wherein m is an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or 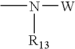
(wherein, W is no atom, carbonyl, carboxylate or amide, R₁₃ is hydrogen, alkyl, cycloalkyl, aryl or heterocycle), R₇ and R₈ together is cycloalkyl, cycloalkenyl, bicyclic alkyl, bicyclic alkenyl, aryl, heterocycle or 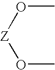
(wherein Z is CO or SO), R₉ and R₁₀ are each independently hydrogen, hydroxy, alkoxy, acetyl, or acetyloxy, R₁₁ (is no atom hydrogen, alkyl, alkenyl, alkynyl, cycloalkyl, aryl, or heterocycle;
X is CO, CS or CHY (wherein Y is hydrogen, hydroxy, halogen, alkoxy or haloalkoxy); and
R is alkyl, alkenyl, alkynyl, cycloalkyl, aryl or heterocycle;
with the provisos that
(i) when A is 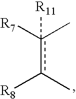
X is —CH₂— and R₁₁ is hydrogen then R₇ is hydrogen or 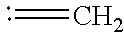
or alkyl with the further proviso that when R₇ is alkyl and R₈ is R₁₂ NH—, then R₁₂ is substituted alkyl wherein the substituents are selected from, aryl or heterocyclyl,
(ii) when A is 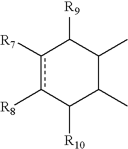
and X is —CH₂—, then none of R₇, R₈, R₉ or R₁₀ are hydrogen or halogen,
(iii) when A is 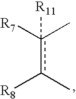
X is —CH₂—, and R₁₁ is no atom, then R₇ can be 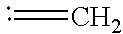
which method comprises the steps of:
(a) reacting a compound of Formula III 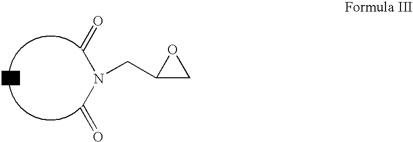
with a compound of Formula VI 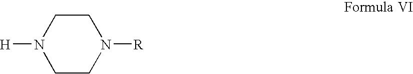
to form a compound of Formula VIII, and 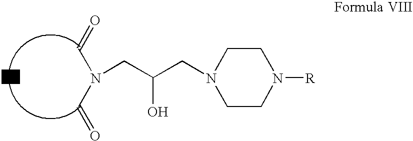
(b) (i) oxidizing a compound of Formula VIII to form a compound of Formula VII, or
(ii) fluorinating a compound of Formula VIII to form a compound of Formula IX.

11. (canceled)

12. (canceled)

13. A method for preparing a compound of Formula XII, 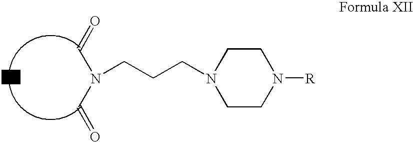
pharmaceutically acceptable salts, pharmaceutically acceptable solvates, enantiomers, diastereomers, n-oxides, prodrugs, polymorphs and metabolites thereof, wherein A is 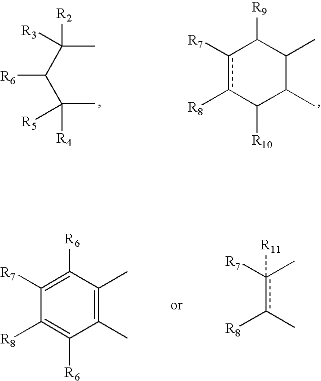
wherein, R₂, R₃, R₄ and R₅ are independently hydrogen, alkyl or phenyl, R₆ is hydrogen, alkyl, phenyl, hydroxy or alkoxy, R₇ and R₈ are each independently hydrogen, alkyl, alkynyl, cycloalkyl, halogen, hydroxy, aryl, acetoxy, heterocycle. 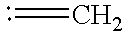
(wherein [symbol] is the point of attachment) or R₁₂-Q-(CH₂)ₘ— (wherein m is an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or 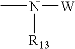
(wherein, W is no atom, carbonyl, carboxylate or amide, R₁₃ is hydrogen, alkyl, cycloalkyl, aryl or heterocycle), R₇ and R₈ together is cycloalkyl,
cycloalkenyl, bicyclic alkyl, bicyclic alkenyl, aryl, heterocycle or 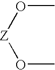
(wherein Z is CO or SO), R₉ and R₁₀ are each independently hydrogen, hydroxy, alkoxy, acetyl, or acetyloxy, R₁₁ is no atom hydrogen, alkyl, alkenyl, alkynyl, cycloalkyl, aryl, or heterocycle;
Z is CO, CS or CHY (wherein Y is hydrogen, hydroxy, halogen, alkoxy or haloalkoxy); and
R is alkyl, alkenyl, alkynyl, cycloalkyl, aryl or heterocycle;
with the provisos that
(i) when A is 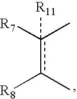
X is —CH₂— and R₁₁ is hydrogen then R₇ is hydrogen or alkyl with the further proviso that when R₇ is alkyl and R₈ is R₁₂ NH—, then R₁₂ is substituted alkyl wherein the substituents are selected from aryl or heterocyclyl,
(ii) when A is 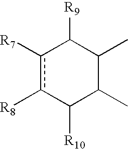
and X is —CH₂—, then none of R₇, R₈, R₉ or R₁₀ are hydrogen or halogen,
(iii) when A is 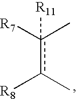
X is —CH₂—, and R₁₁ is no atom, then R₇ can be 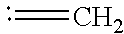
which method comprises:
(a) alkylating a compound of Formula II with a compound of Formula X 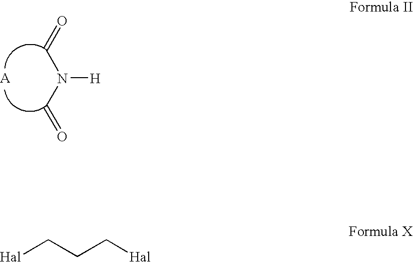
to form a compound of Formula XI 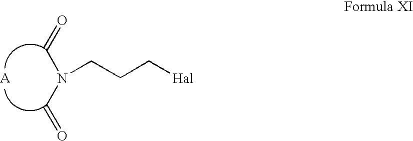
(b) reacting a compound of Formula XI with a compound of VI 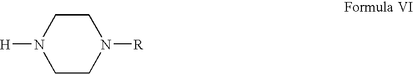
to form a compound of Formula XII.

14. A method for preparing a compound of Formula XVI, 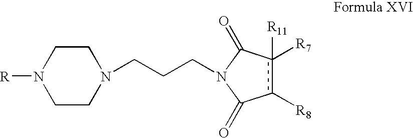
pharmaceutically acceptable salts, pharmaceutically acceptable solvates, enantiomers, diastereomers, n-oxides, prodrugs, polymorphs and metabolites thereof,
wherein R₇ and R₈ are each independently hydrogen, alkyl, alkynyl, cycloalkyl, halogen, hydroxy, aryl, acetoxy, heterocycle, 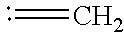
(wherein [symbol] is the point of attachment) or ₁₂-Q-(CH₂)ₘ— (wherein [symbol] is an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or 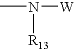
(wherein, W is no atom, carbonyl, carboxylate or amide, R₁₃ is hydrogen, alkyl, cycloalkyl, aryl or heterocycle), R₇ and R₈ together is cycloalkyl, cycloalkenyl, bicyclic alkyl, bicyclic alkenyl, aryl, heterocycle or 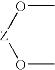
(wherein Z is CO or SO), R₁₁ is no atom hydrogen, alkyl, alkenyl, alkynyl, cycloalkyl, aryl, or heterocycle: and
R is alkyl, alkenyl, alkynyl, cycloalkyl, aryl or heterocycle;
which method comprises:
(a) reacting a compound of Formula VI 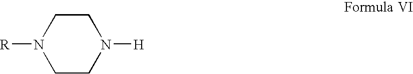
with acrylonitrile to form a compound of Formula XIII, 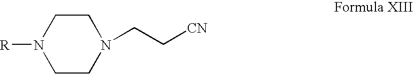
(b) reducing a compound of Formula XIII to form a compound of Formula XIV,
and 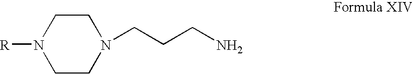
(c) reacting a compound of Formula XIV with a compound of Formula XV 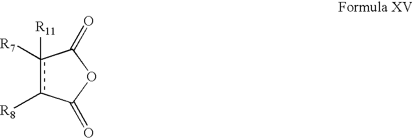
to form a compound of Formula XVI.

15. A method for preparing a compound of Formulae XIX or XXII, 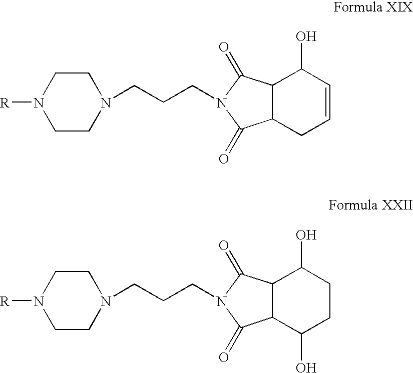
pharmaceutically acceptable salts, pharmaceutically acceptable solvates, enantiomers, diastereomers, N-oxides, prodrugs, polymorphs or metabolites thereof, wherein R is alkyl, alkenyl, alkynyl, cycloalkyl, aryl or heterocycle,
which method comprises the steps of:
(a) (i) reacting a compound of Formula XVII 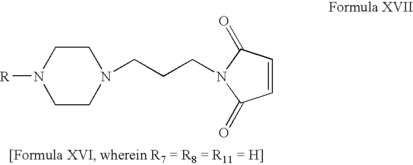
with 1-acetoxy-1,3-butadiene to form a compound of Formula XVIII, and 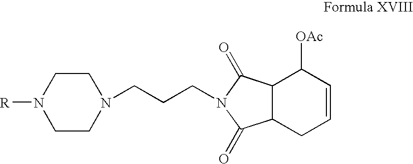
(ii) hydrolyzing a compound of Formula XVIII to form a compound of Formula XIX, or
(b) (i) reacting a compound of Formula XVII 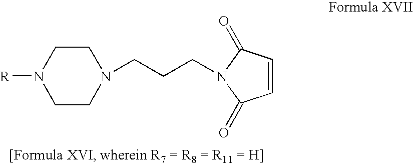
with 1,4-diacetoxy-1,3-butadiene to form a compound of Formula XX, 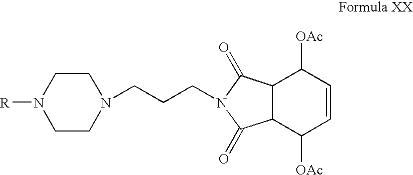
(ii) hydrolyzing a compound of Formula XX to form a compound of Formula XXI, and 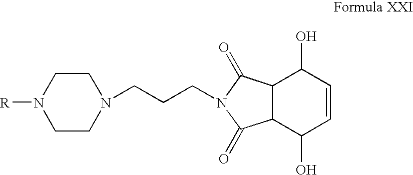
(iii) reducing a compound of Formula XXI to form a compound of Formula XII.

16. (canceled)

17. (canceled)

18. (canceled)

19. (canceled)

20. A method for preparing a compound of Formula XXV, 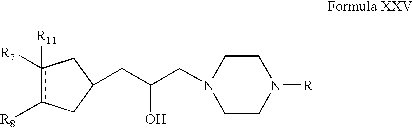
pharmaceutically acceptable salts, pharmaceutically acceptable solvates, enantiomers, diastereomers, n-oxides, prodrugs, polymorphs or metabolites thereof,
wherein R₇ and R₈ are each independently hydrogen, alkyl, alkynyl, cycloalkyl, halogen, hydroxy, aryl, acetoxy, heterocycle, 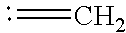
(wherein [symbol] is the point of attachment) or R₁₂-Q-(CH₂)ₘ— (wherein m is an integer of from 0 to 3, R₁₂ can be alkyl, alkenyl, alkynyl, cycloalkyl, cycloalkenyl, aryl, heterocycle, Q can be oxygen, sulfur, carbonyl, carboxylic or 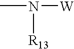
(wherein, W is no atom, carbonyl, carboxylate or amide, R₁₃ is hydrogen, alkyl, cycloalkyl, aryl or heterocycle), R₇ and R₈ together is cycloalkyl, cycloalkenyl, bicyclic alkyl, bicyclic alkenyl, aryl, heterocycle or 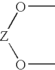
(wherein Z is CO or SO), R₁₁ is no atom hydrogen, alkyl, alkenyl, alkynyl, cycloalkyl, aryl, or heterocycle; and
R is alkyl, alkenyl, alkynyl, cycloalkyl, aryl or heterocycle;
which method comprises:
(a) reacting isoindole-1,3-dione with 2-chloromethyl oxirane to form 2-oxiranylmethyl-isoindole-1,3-dione
(b) reacting 2-oxiranylmethyl-isoindole-1,3-dione with a compound of Formula VI 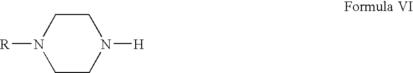
to form a compound of Formula XXIII, 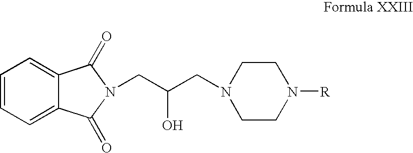
(c) reacting a compound of Formula XXIII with hydrazine hydrate to form a compound of Formula XXIV, and 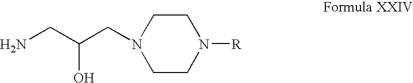
(d) reacting a compound of Formula XXIV with a compound of Formula XV 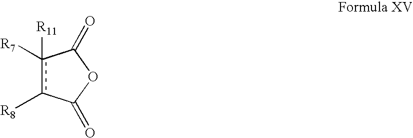
to form a compound of Formula XXV.

21. A method for preparing a compound of Formulae XXVII, XXIX or XXX 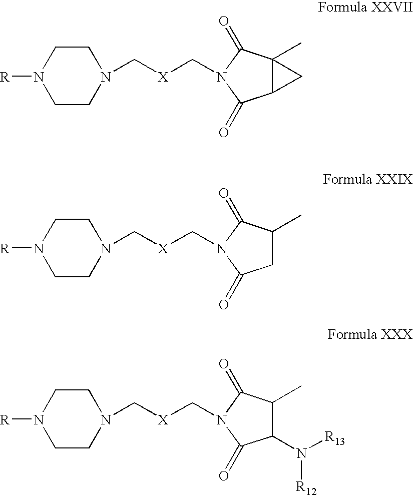
pharmaceutically acceptable salts, pharmaceutically acceptable solvates, enantiomers, diastereomers, n-oxides, prodrugs, polymorphs or metabolites thereof, wherein R is alkyl, alkenyl, alkynyl, cycloalkyl, aryl or heterocycle and X is CO, CS or CHY (wherein Y is hydrogen, hydroxy, halogen, alkoxy or haloalkoxy),
which method comprises the steps of:
(a) reacting a compound of Formula XXVI with a methylating agent 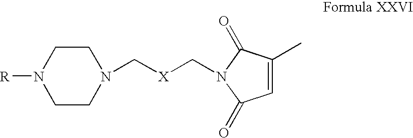
to farm a compound of Formula XXVII, or
(b) reducing a compound[of Formula XXVI to form a compound of Formula XXIX, or
(c) reacting a compound of Formula XXVI with a compound of Formula XXVIII
R₁₂NHR₁₃ Formula XXVIII
to form a compound of Formula XXX.

22. (canceled)

23. (canceled)

24. A method for preparing a compound of Formulae XXXV or XXXVI, 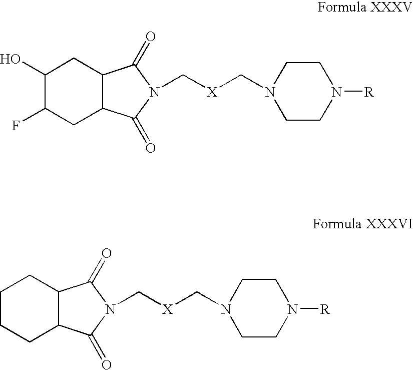
pharmaceutically acceptable salts, pharmaceutically acceptable solvates, enantiomers, diastereomers, N-oxides, prodrugs, polymorphs or metabolites thereof, wherein R is alkyl, alkenyl, alkynyl, cycloalkyl, aryl or heterocycle and X is CO, CS or CHY (wherein Y is hydrogen, hydroxy, halogen, alkoxy or haloalkoxy).
which method comprises the steps of:
reacting a compound of Formula XXXI 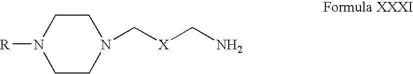
with tetrahydrophthalimide to form a compound of Formula XXXII, and 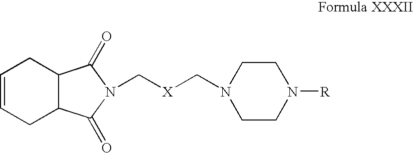
(a) (i) oxidizing a compound of Formula XXXII to form a compound of Formula XXXIII, 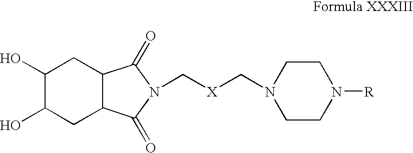
(ii) reacting a compound of Formula XXXIII with diethyl amino sulfur trifluoride to form a compound of Formula XXXIV, and 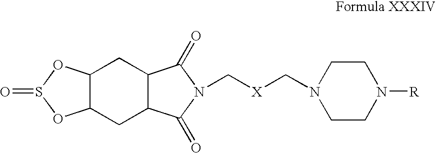
(iii) reacting a compound of Formula XXXIV with diethyl amino sulfur trifluoride to form a compound of Formula XXXV,
or
(b) reducing a compound of Formula XXXII to form a compound of Formula XXXVI.

25. (canceled)

26. (canceled)

27. (canceled)

28. A method for preparing a compound of Formulae XL or XLI, 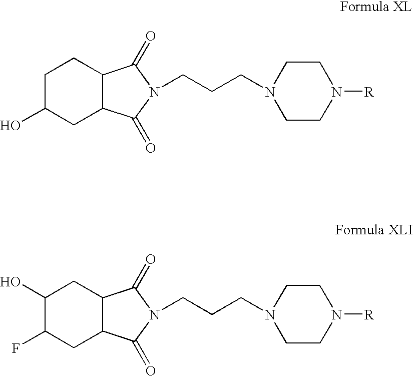
pharmaceutically acceptable salts, pharmaceutically acceptable solvates, enantiomers, diastereomers, N-oxides, prodrugs, polymorphs or metabolites thereof, wherein R is alkyl, alkenyl, alkynyl, cycloalkyl, aryl or heterocycle,
which method comprises the steps of:
(a) reacting a compound of Formula XXXVII 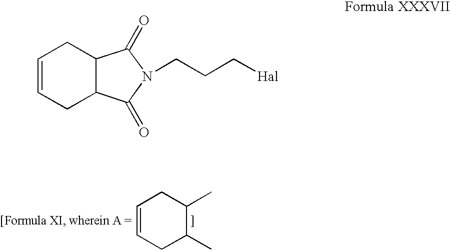
with a peroxy acid to form a compound of Formula XXXVIII, 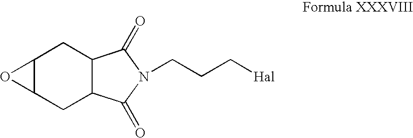
(b) reacting a compound of Formula XXXVIII with a compound of Formula VI 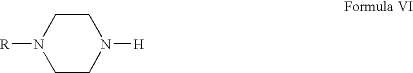
to form a compound of Formula XXXIX, and 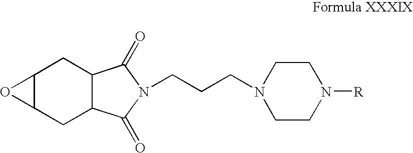
(c) (i) reducing a compound of Formula XXXIX to form a compound of Formula XL,
or
(ii) fluorinating a compound of Formula XXXIX to form a compound of Formula XLI.
